(19) United States
(12) Patent Application Publication
(10) Pub. No.: US 2026/0210976 A1
(43) Pub. Date: Jul. 23, 2026

(54) METHOD TO DETECT AND TREAT PERIPHERAL NEUROPATHY

(71) Applicant: Ohio State Innovation Foundation, Columbus, OH (US)

(72) Inventors: Kristy TOWNSEND, Worthington, OH (US); Magdalena BLASZKIEWICZ, Columbus, OH (US); Jake WILLOWS, Grove City, OH (US)

(21) Appl. No.: 19/140,571

(22) PCT Filed: Dec. 22, 2023
(86) PCT No.: PCT/US2023/085763

§ 371 Date: Jun. 18, 2025

Related U.S. Application Data
(60) Provisional application No. 63/434,979, filed on Dec. 23, 2022.

(51) Int. Cl. G01N 33/68; G01N 33/543

(52) U.S. Cl. CPC G01N 33/6896; G01N 33/54386; G01N 2333/4703; G01N 2474/20; G01N 2800/2842; G01N 2800/52

(57) ABSTRACT

Disclosed herein is a kit to detect and determine post-synaptic density protein-95 (PSD-95) levels in subjects with peripheral neuropathy. Also disclosed herein, is a method of treating or preventing neuropathy in a subject. The method comprises detecting PSD-95 levels in a sample from the subject, using the kit disclosed herein and when the PSD-95 level differs from a control, the subject is treated for neuropathy.

### CROSS-REFERENCE TO RELATED APPLICATIONS

This application claims priority to, and the benefit of, U.S. Provisional Patent Application Ser. No. 63/434,979, filed Dec. 23, 2022, the disclosure of which is expressly incorporated herein by reference in its entirety.

### GOVERNMENT SUPPORT CLAUSE

This invention was made with government support under grant/contract number R01 DK114320 awarded by the National Institutes of Health. The government has certain rights in the invention.

### REFERENCE TO SEQUENCE LISTING

The sequence listing submitted on Dec. 22, 2023, as an .xml file entitled “103361-305WO01_ST26.xml” created on Dec. 21, 2023, and having a file size of 76,424 bytes is hereby incorporated by reference pursuant to 37 C.F.R. § 1.52 (e) (5).

### BACKGROUND

Small fiber neuropathy is a condition characterized by severe pain attacks that typically begin in the feet or hands. In many affected individuals, such neuropathies involve the small nerve fibers, including the peripheral thinly myelinated A8 fibers as well as unmyelinated C nerve fibers (Devigili 2020). Involvement of these small nerve fibers, referred to as small fiber neuropathy (SFN), typically presents with pain, burning, numbness, and tingling, often in a stocking-glove distribution, with symptoms typically starting in the feet and ascending superiorly (Cascio 2023).

Small fiber neuropathy affects the small myelinated A8-fibers as well as the unmyelinated C-fibers (Hovaguimian 2011). SFN can affect both sensory and autonomic fibers, leading to sensory changes, autonomic dysfunction, or a combination of symptoms (Lacomis 2002). General symptoms of SFN include fatigue, cognitive disturbances, headache, and widespread musculoskeletal pain, and thus may negatively impact their quality of life. SFN is associated with a multitude of diseases.

Aging is the predominant risk factor for disease and is associated with numerous comorbidities that are linked to peripheral nervous system function and metabolic control (Niccoli & Partridge, 2012). For example, with aging there is increased incidence of peripheral neuropathy (Brisset & Nicolas, 2018), cardiovascular disease (North & Sinclair, 2012), and type 2 diabetes mellitus (Kirkman et al., 2012). With aging there is also a redistribution of adipose tissue with more ectopic lipid deposition in heart, muscle, and liver, as well as reduced capacity for browning (development of uncoupling protein 1 (UCP1)-expressing brown adipocytes in white adipose tissue (WAT) depots), and increased inflammation of adipose tissues (Palmer & Kirkland, 2016). WAT is a highly plastic organ, capable of remodeling and changing tissue composition in response to metabolic demands. For example, the tissue can increase cell size or cell number (hypertrophy and hyperplasia, respectively) and initiate browning in response to various stimuli such as cold stimulation (a process that requires mitochondrial biogenesis and neurovascular remodeling). Therefore, aging represents a loss of these remodeling capabilities, driven by pathophysiological changes to the tissue such as inflammation, fibrosis, and neuropathy (Blaszkiewicz et al., 2019; Khan et al., 2009; Palmer & Kirkland, 2016).

Loss of proper tissue and organ innervation with aging-related peripheral neuropathy can also underlie many of the observed phenotypes of aging, since the nervous system is known to regulate adipose tissue lipolysis, muscle function, and other metabolically relevant processes.

What is needed in the art are effective methods of diagnosing and treating peripheral neuropathy, and for kits comprising tools to do so.

### SUMMARY

The present invention relates to a kit for detecting and a method for treating neuropathy and use of the kit thereof. The present disclosure addresses at least a portion of the problems described above through the use of the inventive kit and methods of treating neuropathy.

In one aspect, the present invention provides a kit for determining an amount of post-synaptic density protein-95 (PSD-95) present in a subject. In some embodiments, the kit comprises a means for detecting PSD-95. In some embodiments, a change in the amount of PSD-95 is reflective of a change in synaptic density within the subject. In some embodiments, the means for detecting PSD-95 is a probe, wherein the probe detects a protein or nucleic acid. In some embodiments, the probe is an antibody. In some embodiments, the probe is a nucleic acid. In some aspects, the means for detecting PSD-95 is a set of primers. As disclosed herein, the kit further comprises a testing well, a secondary fluorescent detection agent, a wash buffer or extraction buffer, a signal amplification buffer, or a combination thereof. In some embodiments, the testing well is a 96-well plate. In some embodiments, a probe is coated onto the base of a testing well. In other aspects, the kit further comprises a circular blade. In some embodiments, the means comprises an Enzyme-Linked Immunosorbent Assay (ELISA) and in some embodiments, the means comprises a Polymerase Chain Reaction (PCR) assay. In further aspects, the PCR assay comprises, a polymerase enzyme, nuclease-free water, MgCl₂, deoxynucleotide triphosphates (dNTPs), or a combination thereof. The kit further comprises additional means for detecting any additional marker. In some embodiments, the additional marker comprises one or more of PGP9.5, CGRP, GAP43 and tyrosine hydroxyl.

In another aspect, the present invention provides a method of treating or preventing peripheral neuropathy or changes to peripheral innervation in response to disease, injury, treatments, or therapies in a subject. In some embodiments, the method comprises detecting PSD-95 levels in a sample from the subject, wherein, when the PSD-95 level differs from a control, the subject is treated for neuropathy. In some embodiments, the control is a measurement at a different timepoint from the subject themselves. In further aspects, the sample is derived from adipose samples or interstitial fluid, or other bodily fluid samples. In some aspects, samples are from skin or are subdermal.

Also disclosed herein, after detection of PSD-95 levels, is the method of treatment comprising modulating adipose number and/or size by using a therapy selected from a group comprising calorie-restricted diets/exercise, cholesterol-free diets, low-fat diets, the use of bioactive compounds, pharmacological compounds, or a combination thereof. In some embodiments, the method of treating or preventing symptoms in a subject comprises administering a PSD-95 modulator to the subject at a therapeutically effective dose. In other embodiments, the subject is treated with a pharmacological MMP-13 inhibitor at a therapeutically effective dose. Also disclosed herein, after treatment, effectiveness of treatment can be assessed, wherein PSD-95 levels are measured to determine if treatment was effective, using the kit disclosed in this invention. In some embodiments, the effectiveness of the treatment is validated by immunostaining to measure PSD-95 levels in one target selected from the group comprising nerve fiber density, Intradermal N, number of terminal Schwann cells in a neuromuscular junction, adiposity, adipose cell size, adipose tissue inflammation. In some embodiments, the subject is human.

Additional aspects and advantages of the disclosure will be set forth, in part, in the detailed description and any claims which follow, and in part will be derived from the detailed description or can be learned by practice of the various aspects of the disclosure. The advantages described below will be realized and attained by means of the elements and combinations particularly pointed out in the appended claims. It is to be understood that both the foregoing general description and the following detailed description are exemplary and explanatory only and are not restrictive of the disclosure.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 (A-N) shows genetic background influences on adiposity with aging. Male C57BL/6J (BL6) mice at 15 weeks and 75 weeks of age were compared for body weight (A). Subcutaneous white adipose tissue (scWAT) and perigonadal (pg) WAT weights were used to calculate subcutaneous adiposity (scWAT/body weight) (B), and visceral adiposities (pgWAT/body weight) (C). Male and female HET3 mice at 13 weeks, 30 weeks, 41 weeks, 62 weeks, 106 weeks, and 126 weeks were compared by age or sex for body weight (D-E), subcutaneous adiposity (F-G), and visceral adiposity (H-I). Hematoxylin staining was performed on BL6 mouse axillary (ax)-scWAT and pgWAT (J) and cell sizes were quantified by area and perimeter (K). HET3 pgWAT was stained with hematoxylin (L) and cell size was quantified for males (M) and females (N). Three representative images were captured per tissue, quantified, and averaged per tissue per animal (J-N). BL6 mice; N=3-7. HET3 mice; N=5-12. Unpaired two-tailed Student's t-test (A-C, K). Two-way ANOVA with Tukey's correction for multiple comparisons (D-I). One-way ANOVA with Tukey's correction for multiple comparisons (M-N). Error bars are SEMs. ^{n.s.}p>0.05, *p<0.05, **p<0.01, ***p<0.001, ****p<0.0001.

FIG. 2 (A-J) shows age-related neuropathy. Von Frey tactile allodynia test was performed on male BL6 mice at 15 weeks and 75 weeks (A) and both male and female HET3 mice at 62 weeks and 126 weeks (B). Compared by age and/or sex at each filament strength as well as for the area under each curve. Male HET3 mice at 35 weeks, 65 weeks, and 95 weeks had hind paw skin assessed for intraepidermal nerve fiber (IENF) density via immunofluorescent staining and confocal imaging. Peripheral nerves (PGP9.5) and nuclei (DAPI) (C). Medial gastrocnemius and soleus muscles were stained for neuromuscular junction (NMJ) occupation nerve/pre-synapse (SV2, 2H3), post-synapse (BTX) (D). NMJ occupation was quantified and compared by age; 50 NMJs were counted per tissue (E). At all ages NMJs were co-stained with markers for myelination (MPZ) (F) and Schwan cells (S100B) (G). Representative images of occupied and unoccupied NMJs displayed (F-G). BL6 mice; N=7. HET3 mice; N=5-12. Unpaired two-tailed Student's t-test (A). Two-way ANOVA with Tukey's correction for multiple comparisons (A-B). One-way ANOVA with Tukey's correction for multiple comparisons (E). Error bars are SEMs. ^{n.s.}p>0.05, *p<0.05, **p<0.01, ***p<0.001, ****p<0.0001. a=male-62 weeks: male-126 weeks; b=female-62 weeks: female-126 weeks; c=male-62 weeks: female-62 weeks; d=male-126 weeks: female-126 weeks.

FIG. 3 (A-E) shows Adipose tissue and skin gene expression changes across aging. Gene expression by qPCR in axillary scWAT from male BL6 mice (a). Gene expression in axillary scWAT of HET3, males (b) and females (c). Gene expression in axillary scWAT of HET3 in males (b) and females (c). Gene expression of HET3 flank skin in males (d) and females (e). Genes organized into functionally similar groups: vasculature (VA), cytokines (CK), Schwann cell (SC), synaptic (SY), cellular respiration (CR), and collagen (CO). BL6 mice; N=6-7. HET3 mice; N=5-7. Unpaired two-tailed Student's t-test (a). Kruskal-Wallis nonparametric test with Dunn's post hoc for multiple comparisons, corrected p-values reported (b-d). Error bars are SEMs. p>0.05, *p<0.05, **p<0.01, ***p<0.001, ****p<0.0001. a=30 weeks: 60 weeks, b=30 weeks: 126 weeks.

FIG. 4 (A-P) shows adipose tissue collagen distribution across aging. Picrosirius red (PSR) collagen staining and quantification of 7 μm thick adipose tissue sections. Male BL6 scWAT (A-D). Male BL6 pgWAT (E-H). Male HET3 scWAT (I-L). Male and female HET3 pgWAT (M-P).

Representative images of the same regional tissue area were captured separately with brightfield and polarized light at 20× objective magnification (A, E, I, M). Five representative images were captured per tissue per animal. Total collagen was measured as a ratio of birefringent collagen to total PSR staining (B, F, J, N). Changes in specific hues of collagen birefringence (C, G, K, O). Contribution of collagen fiber thickness determined by hue (D, H, L, P). Thin collagen fibers (green and yellow); thick collagen fibers (orange and yellow). BL6 mice; N=3-7. HET3 mice; N=3-6. Unpaired two-tailed Student's t-test (B, D, F, H, J, L). Two-way ANOVA with Tukey's correction for multiple comparisons (C, G, K, N, O). One-way ANOVA with Tukey's correction for multiple comparisons (P). All error bars are SEMS p>0.05, *p<0.05, **p<0.01, ***p<0.001, ****p<0.0001.

FIG. 5 (A-I) shows vascular changes with aging. Wire myography of male HET3 aortic vasoconstriction and (a) and vasorelaxation (b) at 30, 60, and 80 weeks (N=3-9). Segments of thoracic aorta were contracted with a dose response of phenylephrine. Vessels were then returned to basal tone, and pre-contracted to 50%-80% maximal phenylephrine-induced contraction. Dose response curve of acetylcholine was performed to measure vasorelaxation (a). Vasocontraction was normalized to maximal KCl contraction, and vasorelaxation was calculated as percentage of Pre-contraction (B). EC50 for contraction and relaxation were calculated (a,b). Intact inguinal scWAT depots were excised from male HET3 mice at 20, 60, and 100 weeks and labeled for nerves (tyrosine hydroxylase, TH) and blood vessels (isolectin IB4, IB4). Micrographs of whole tissues were generated from 10× objective magnification confocal images that were tiled together and Z-maximum intensity projected (c). Representative images of N=5 tissues are displayed (c). Relative nerve fiber density for the whole tissue was calculated as TH-labeled area normalized to total tissue area (d). Relative vascular density for the whole tissue was calculated as IB4-labeled area normalized to total tissue area (e). Relative neurovascular area was calculated as TH area normalized to IB4 area (f). Higher magnification representative maximum intensity projection images were captured at 10× objective magnification or 63× objective magnification with an additional 2.50× confocal zoom applied (g). Colocalization of nerve-blood vessel overlap was performed by comparing Mander's coefficients between groups. Higher values correspond to greater overlap. Overlap was calculated from the 10× objective magnification images of the intact whole tissues (N=4-6, n=5) (h,i). Scale bars are 10 mm (c), 200 μm and 20 μm (g). One-way ANOVA with Tukey's correction for multiple comparisons. All error bars are SEMs. p>0.05, *p<0.05, **p<0.01, ***p<0.001, ****p<0.0001.

FIG. 6 (A-C) shows neuro-adipose nexus (NAN) distribution fluctuates with age. Intact inguinal scWAT depots were excised from male HET3 mice at 20, 60, and 100 weeks and labeled for nerves with TH. NANs were identified by densely varicose axons innervating single adipocytes (visualized with autofluorescence). NAN morphology compared across ages (a). NAN distribution across whole ing-scWAT depots displayed as a representative tissue from each age group. Individual NANs labeled by a green dots superimposed over the intact tissue (b). Adjacent dot overlap was color coded and displayed below (b). Total number of NANs was counted for each tissue (N=4-6) (c). Scale bars are 30 μm (a) and 10 mm (b). One-way ANOVA with Tukey's correction for multiple comparisons. All error bars are SEMs. p>0.05, *p<0.05, **p<0.01, ***p<0.001, ****p<0.0001.

FIG. 7 (A-S) shows rapamycin treatment had no effect on age-related neuropathy in HET3 mice. Male and female HET3 mice were diet-fed rapamycin (42 ppm) (Rapa) or received standard chow (Veh) for 8 months. Treatment was either started at 30 weeks (early-intervention) or at 72 weeks (late intervention). Body weights were recorded through duration of treatment (A-B). Subcutaneous (C-D) and visceral (E-F) adiposity, and quad weight (G-H) at time tissue collection. Hematoxylin/Hemalum staining of scWAT (I-J). Von Frey tactile allodynia assay with area under each curve quantified (K-L). Medial gastrocnemius and soleus muscles were stained for NMJ occupation, nerve/pre-synapse (SV2, 2H3), post-synapse (BTX). Representative images of medial gastrocnemius muscles (M). White boxes are digital zoom-ins of NMJs, and white arrows mark unoccupied NMJs (M). NMJ occupation was quantified for medial gastrocnemius and soleus for early-(N) and late-intervention groups (O). Gene expression of axillary scWAT was measured by qPCR (P-S). Genes organized into functionally similar groups: vasculature (VA), cytokines (CK), Schwann cell (SC), synaptic (SY), cellular respiration (CR), and collagen (CO). N=3-12. Two-way ANOVA with Tukey's correction for multiple comparisons (A-H, K-L, N—O). Mann-Whitney nonparametric test (P-S). All error bars are SEMS. ^{n.s.}p>0.05, *p<0.05, **p<0.01, ***p<0.001, ****p<0.0001. a=male-veh: male-rapa; b=female-veh: female-rapa; c=male-veh: female-veh; d=male-rapa: female-rapa.

FIG. 8 (A-K) shows rapamycin treatment started early in life increased scWAT fibrosis. PSR staining of scWAT from early-intervention (A-F) versus late-Intervention (G-K) of rapamycin (42 ppm) treated male and female HET3 mice imaged at 4× objective magnification (A, G). Five representative images of tissue parenchyma were captured separately with brightfield and polarized light at 20× objective magnification per tissue per animal (B, H). Total collagen was measured as a ratio of birefringent collagen to total PSR staining (C, I). Changes in specific hues of collagen birefringence (D, J). Contribution of collagen fiber thickness determined by hue (E, K). Thin collagen (green and yellow); Thick collagen (orange and yellow). N=3-11. Two-way ANOVA with Tukey's correction for multiple comparisons (C-D, I-J). One-way ANOVA with Tukey's correction for multiple comparisons (E, K). All error bars are SEMs. p>0.05, *p<0.05, **p<0.01, ***p<0.001, ****p<0.0001.

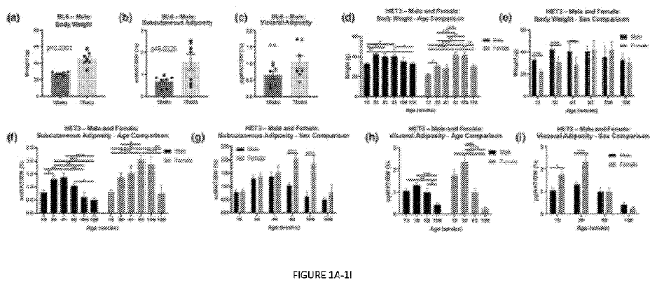

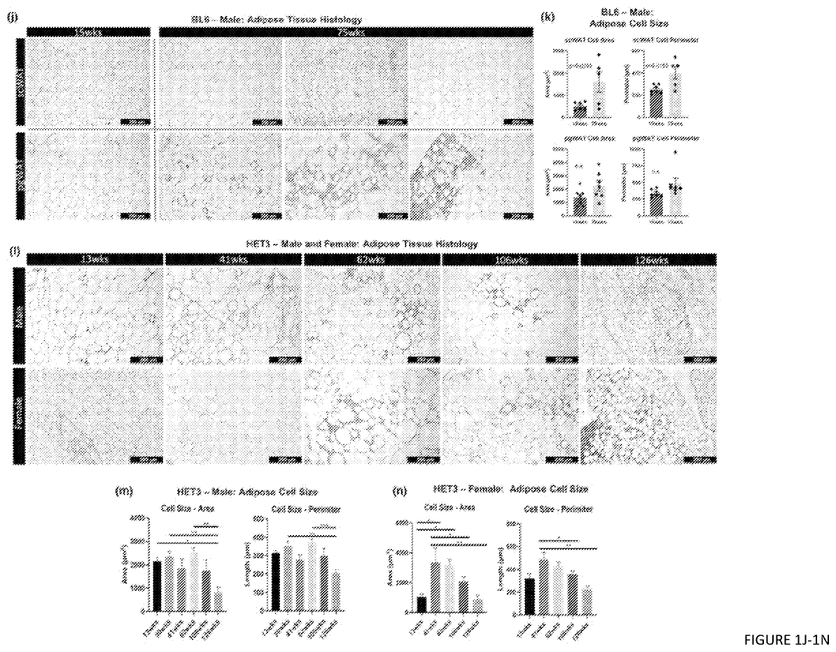

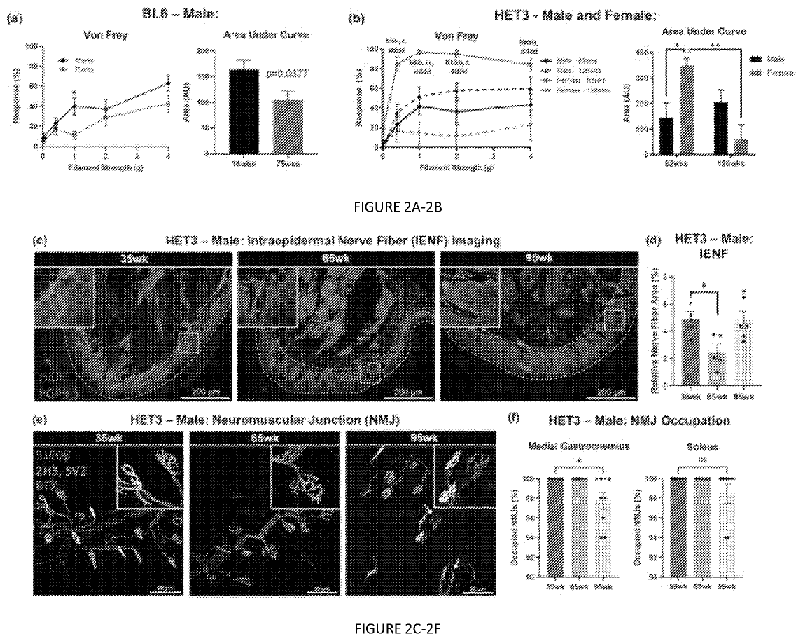

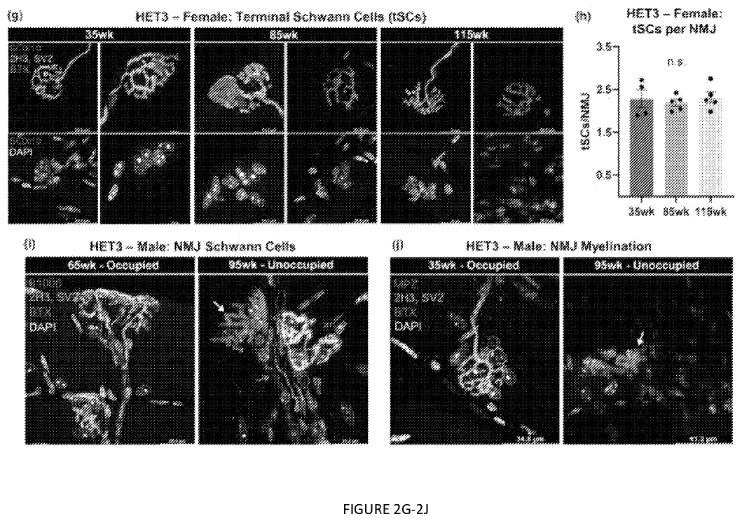

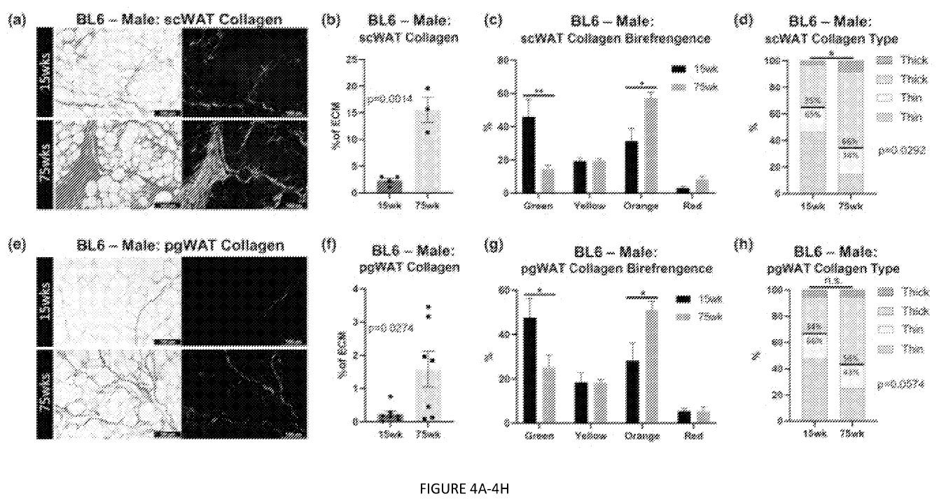

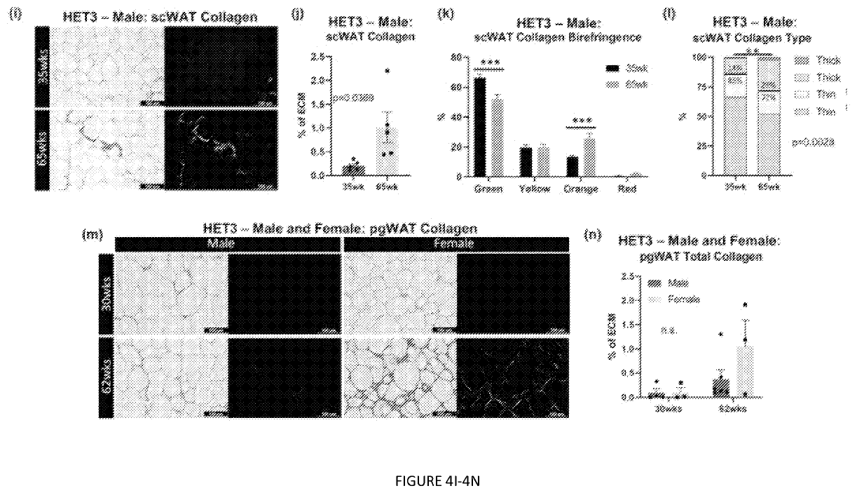

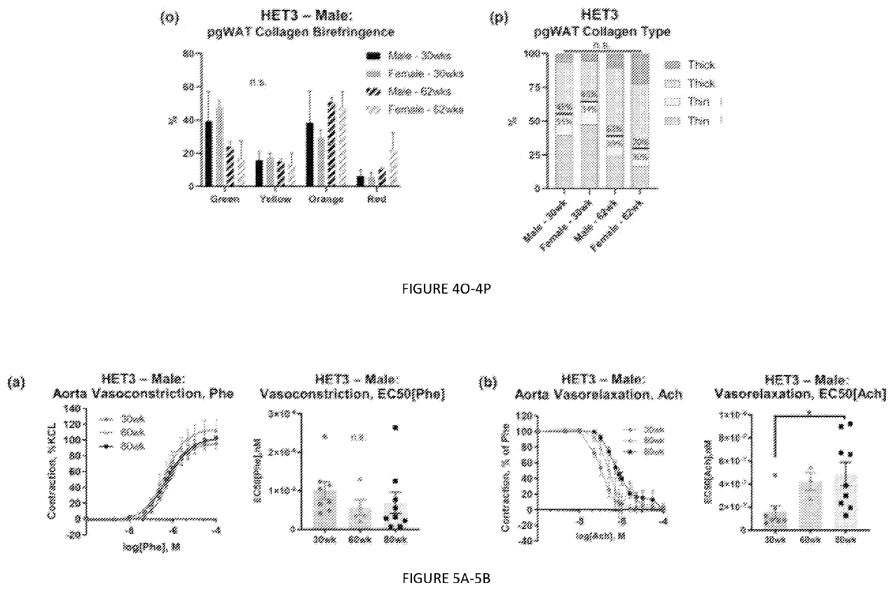

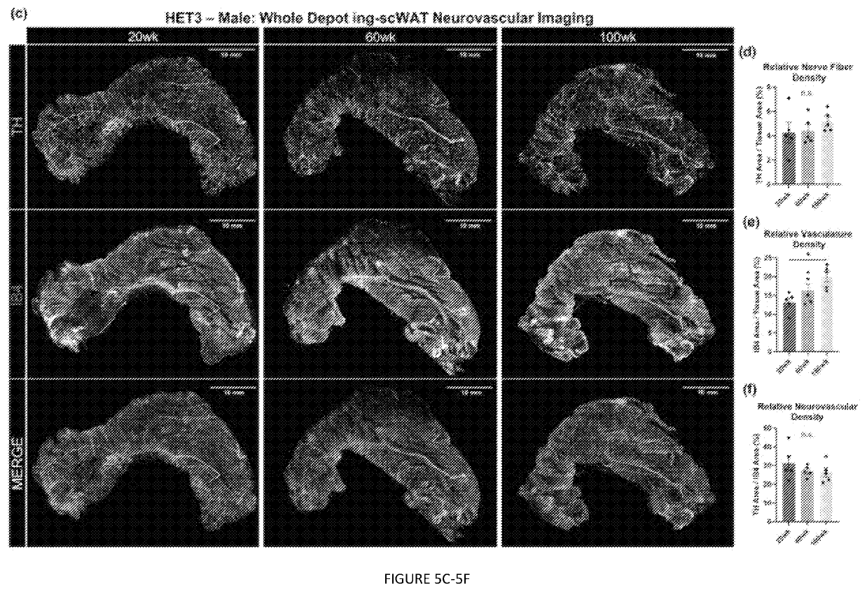

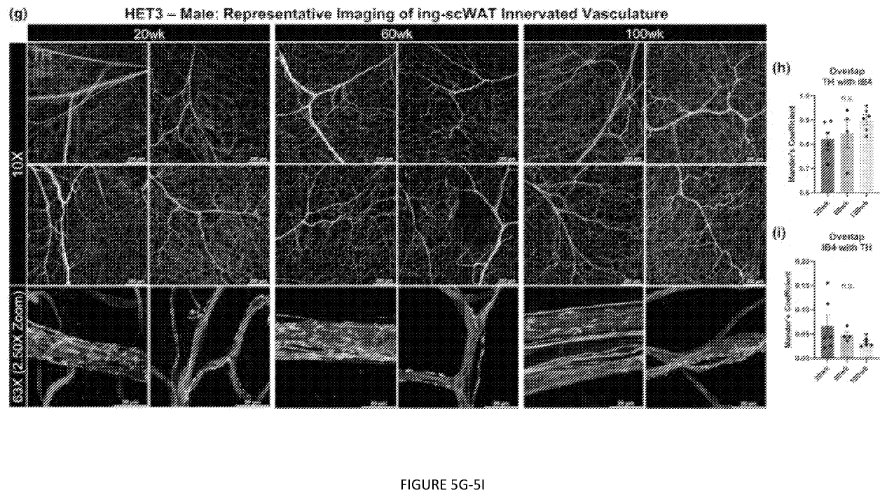

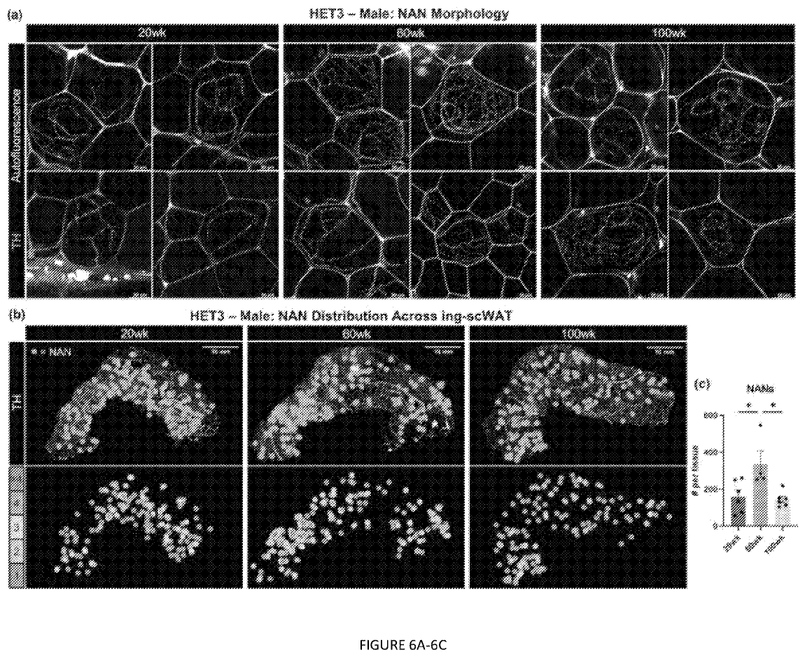

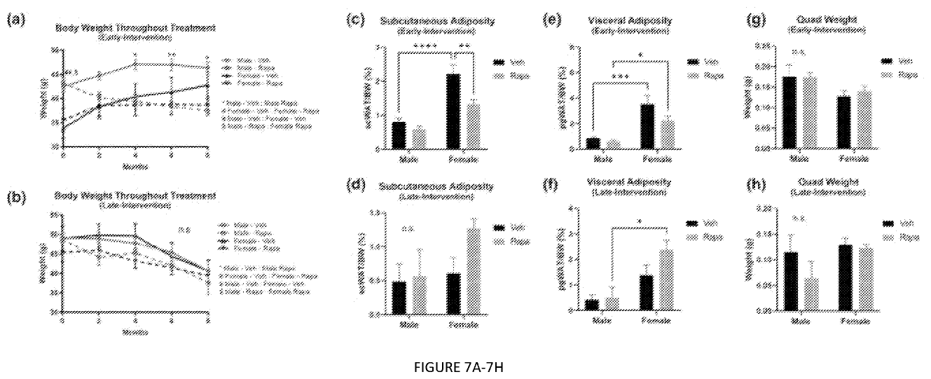

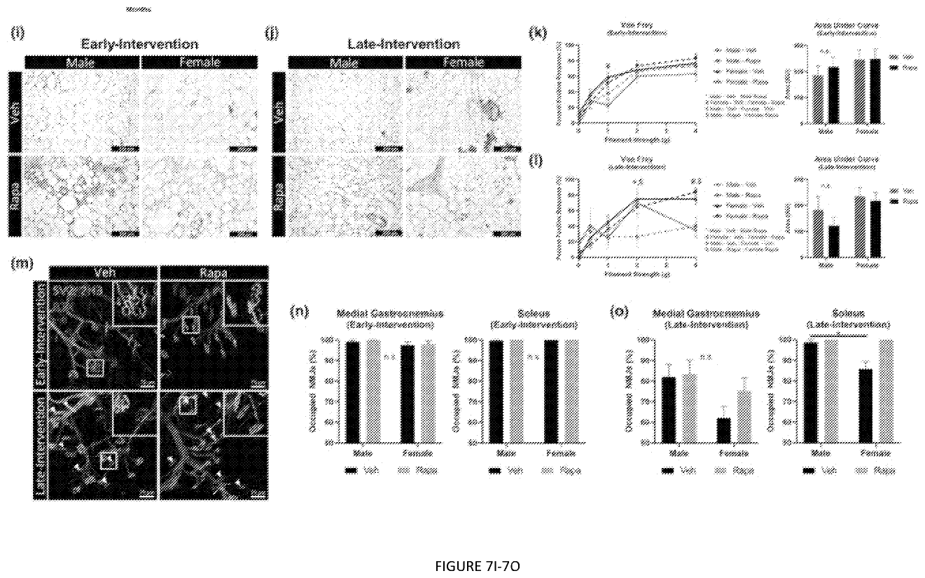

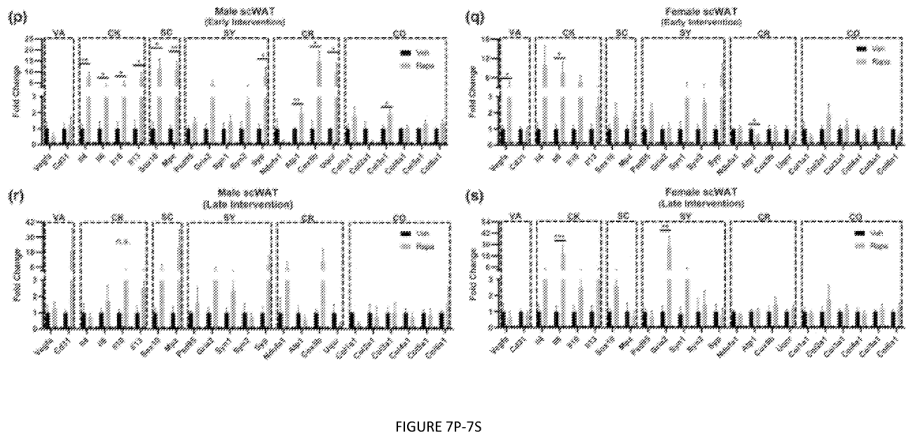

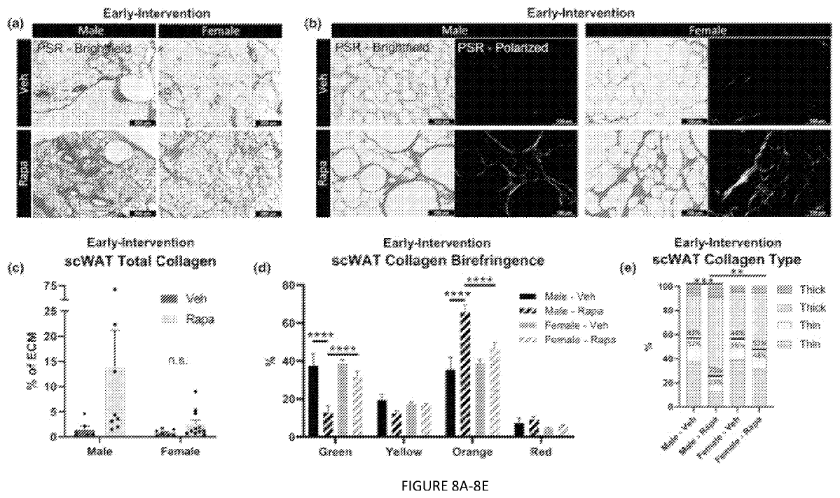

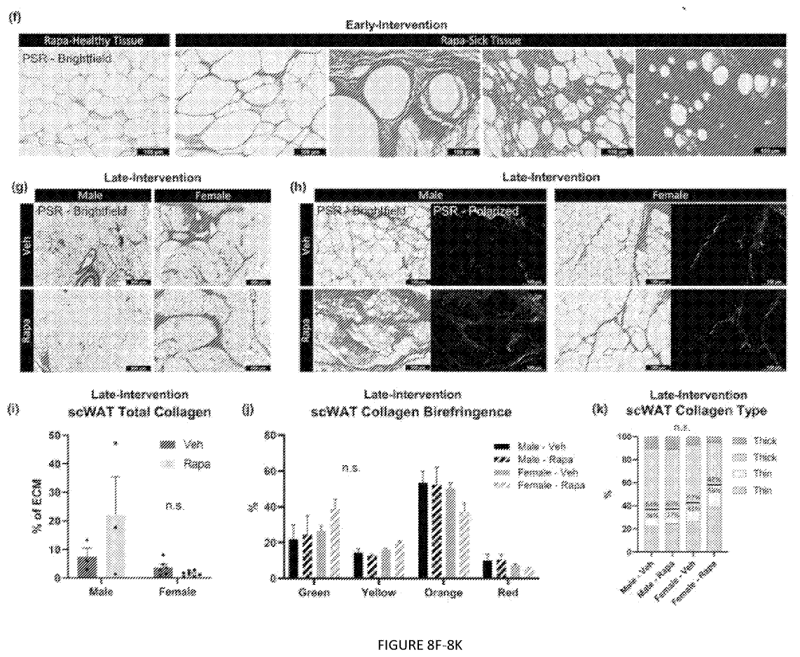

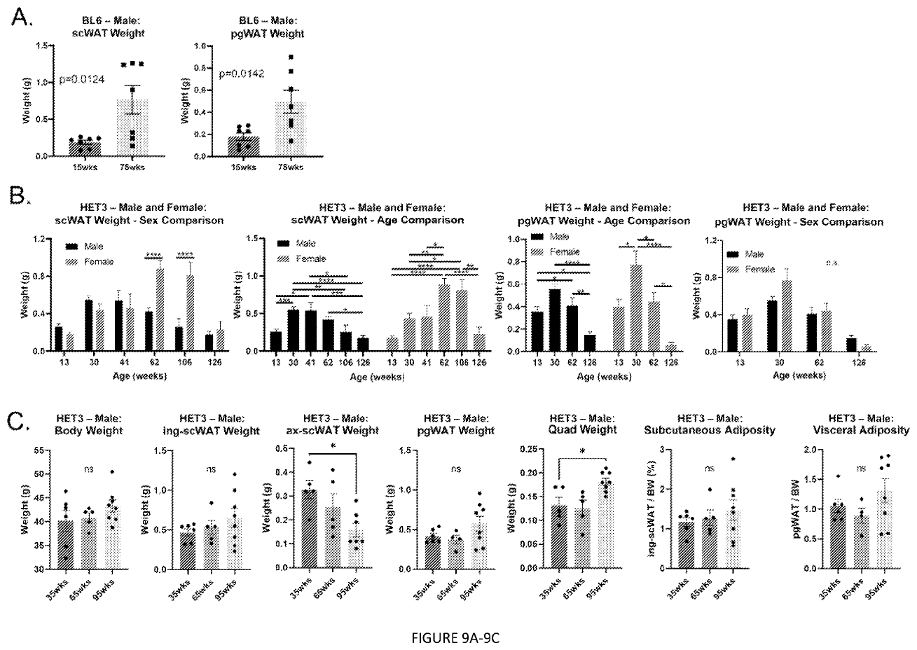

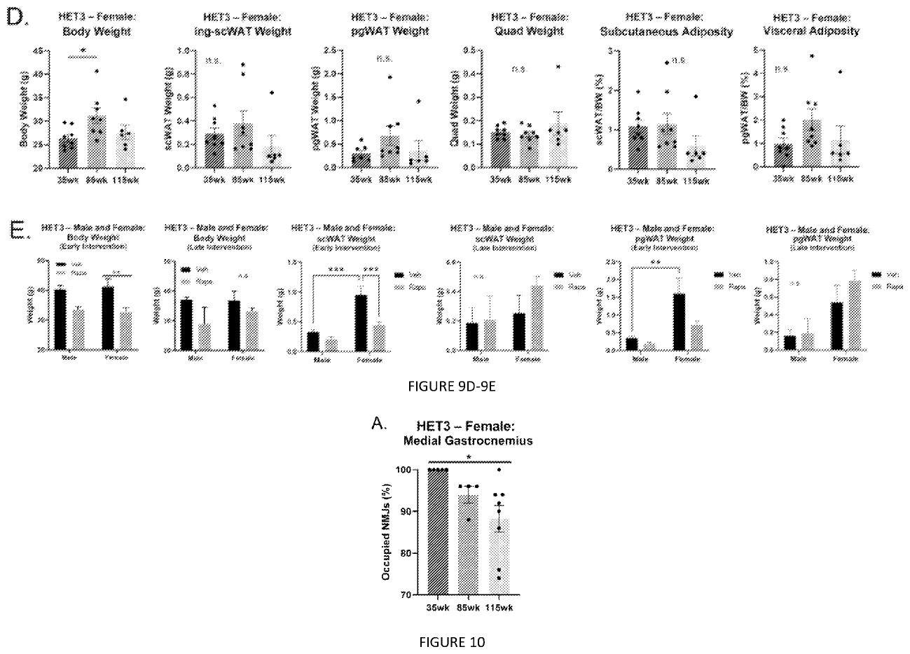

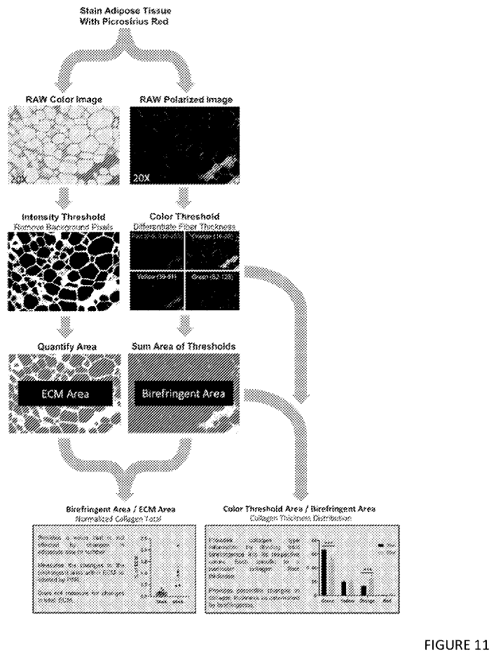

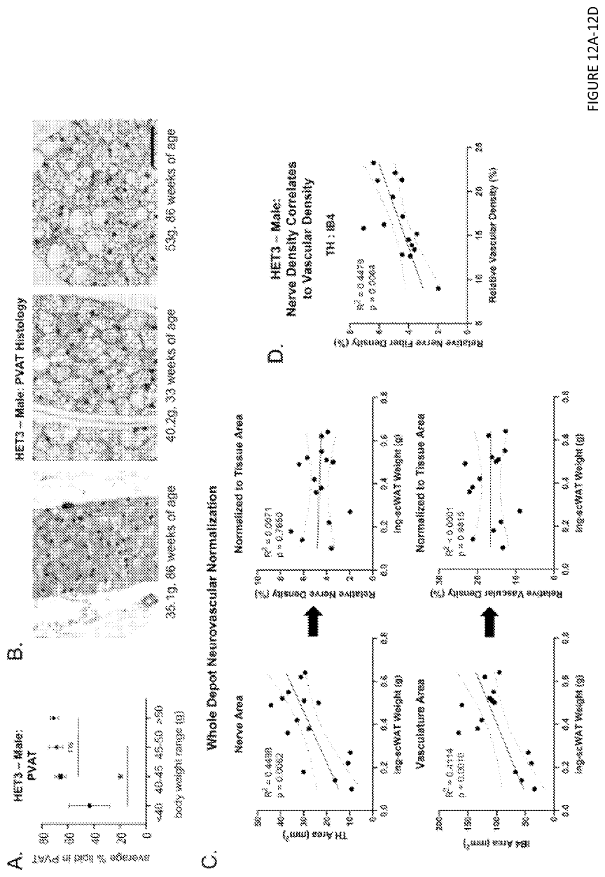

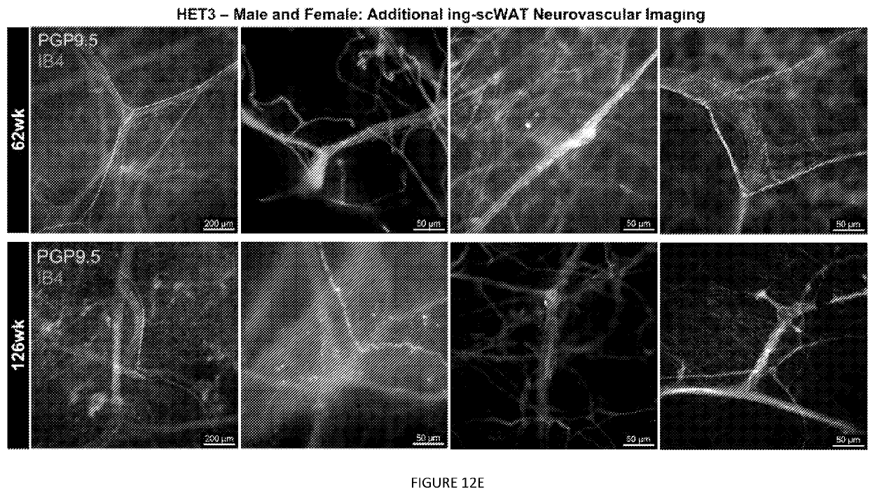

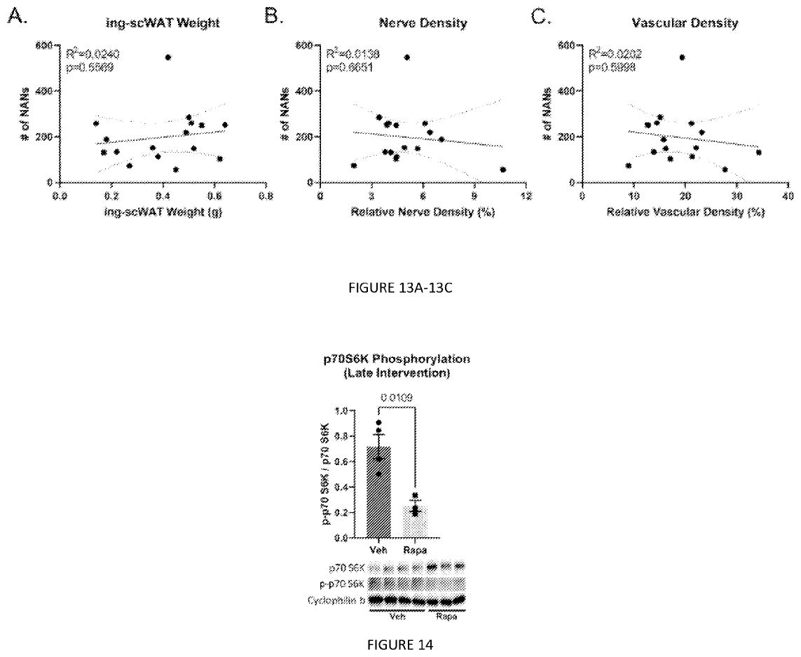

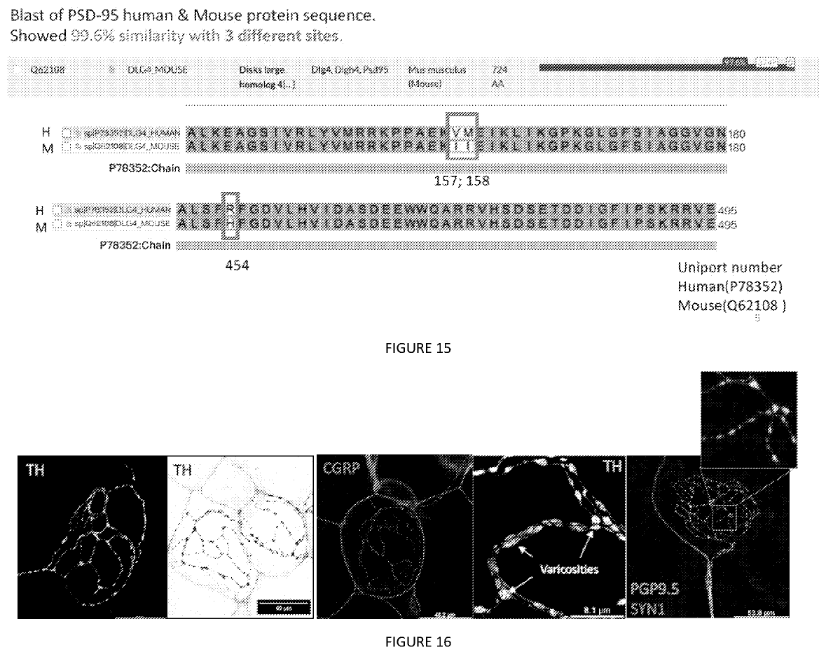

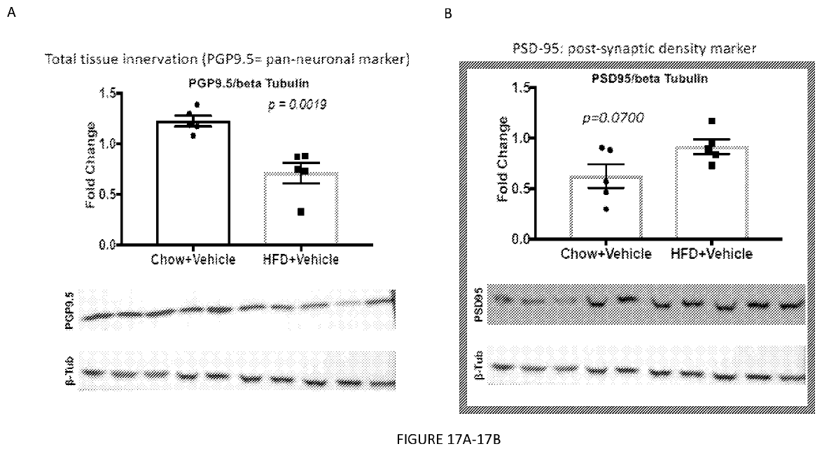

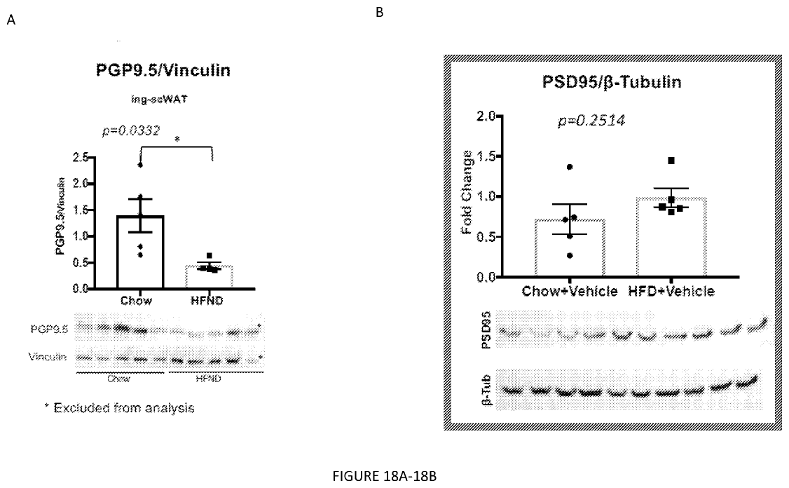

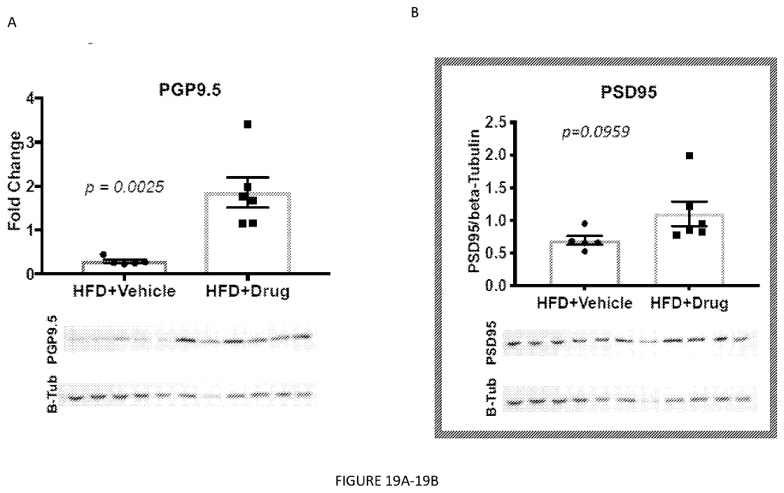

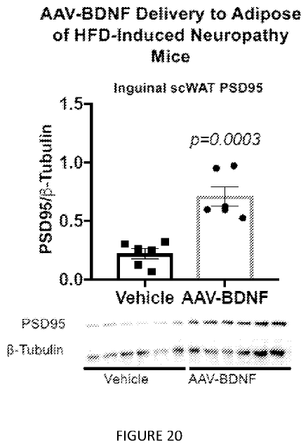

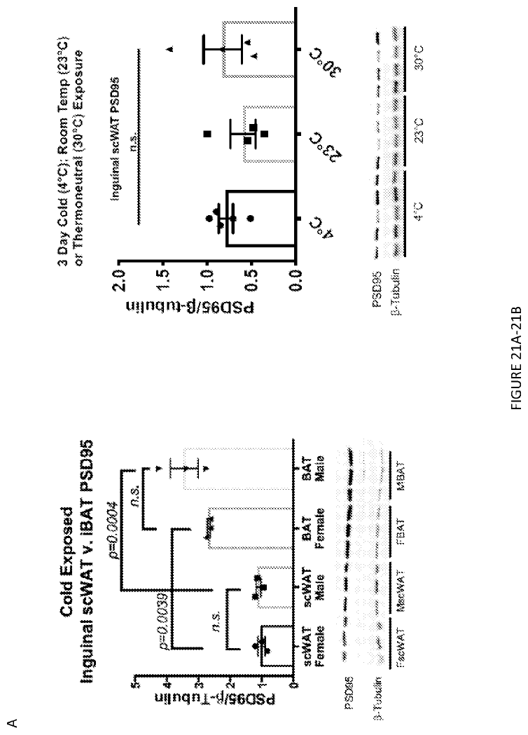

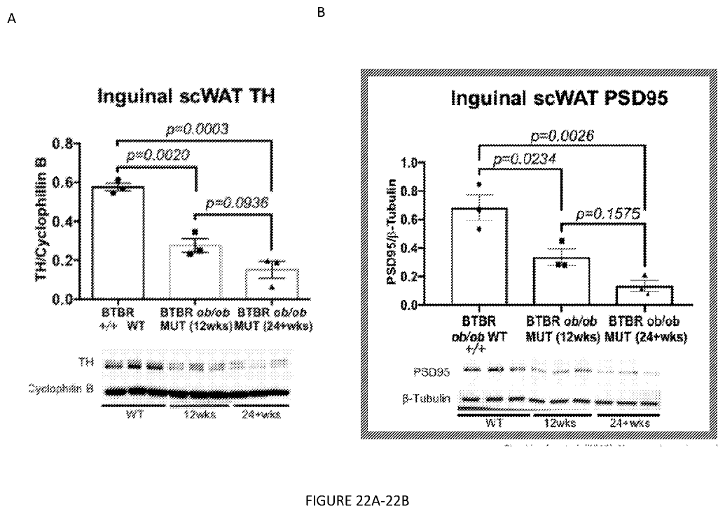

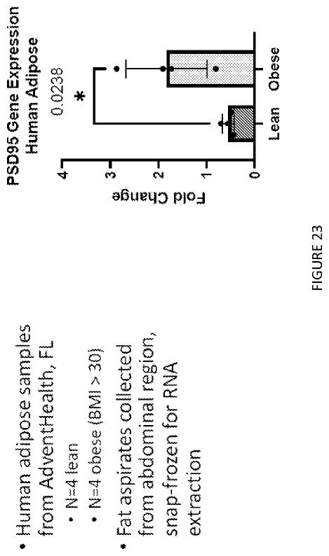

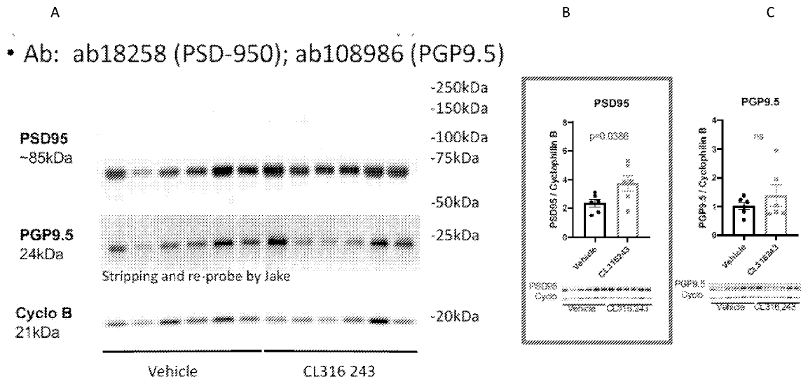

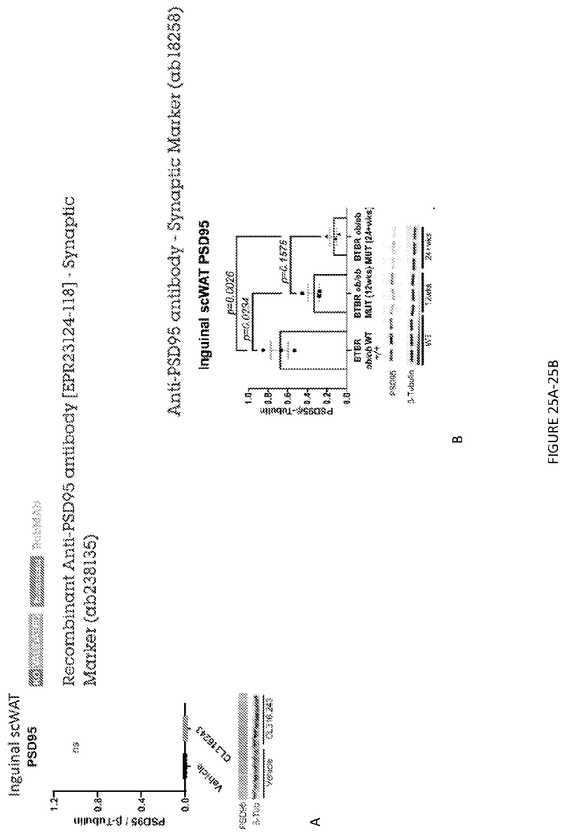

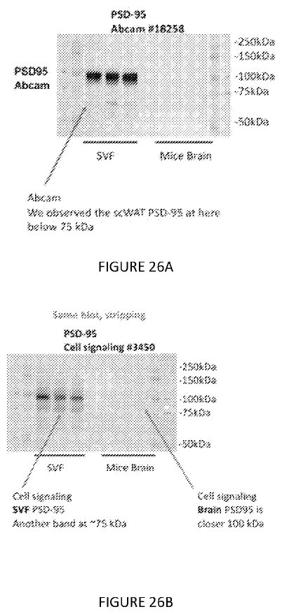

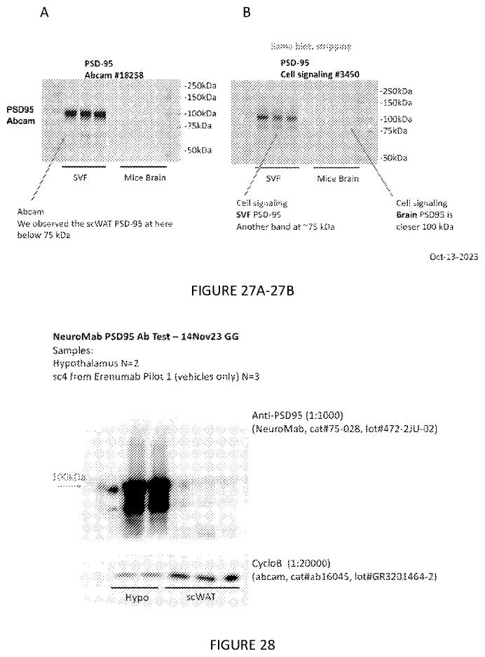

FIG. 9 (A-E) shows additional weight data. C57BL/6J (BL6) (A) and HET3 (B) adipose depot weights used to quantify adiposity in FIG. 1. Male HET3 Replicate weights and adiposities related to animals presented in FIGS. 2 and 4 (C). HET3 body and adipose depot weights are used to calculate adiposities in FIG. 6 (D-E). BL6 mice; N=7. HET3 mice; N=5-12. Unpaired two-tailed Student's t-test (A). Two-way ANOVA with Tukey's correction for multiple comparisons (B, D). One-way ANOVA with Tukey's correction for multiple comparisons (C). Error bars are SEMs. p>0.05, *p<0.05, **p<0.01, ***p<0.001, ****p<0.0001.

FIG. 10 shows age-related NMJ neuropathy in female HET3 mice. Medial gastrocnemius muscles from female HET3 mice at 35 weeks, 85 weeks, and 115 weeks were immunolabeled to assess NMJ occupation. Occupation was assessed for 50 NMJs per tissue (N=4-8). Related to data presented in FIG. 2G-H. One-way ANOVA with Tukey's correction for multiple comparisons (C). Error bars are SEMs. ^{n.s.}p>0.05, *p<0.05, **p<0.01, ***p<0.001, ****p<0.0001.

FIG. 11 shows the method for quantifying adipose tissue collagen. A diagram depicting the method used to quantify total adipose tissue collagen and distribution of thin and thick collagen fibers in adipose tissue that is not affected by adipocyte size or number. Hue discrimination was adapted from (Rich & Whittaker, 2005). Related to data presented in FIGS. 4 and 7.

FIG. 12 (A-E) shows Perivascular adipose tissue characterization and ing-scWAT neurovascular correlations. Lipid content was quantified in hematoxylin/eosin stained slides of mouse PVAT (Tero et al., 2022). Mice were categorized into body weight <40 g (N=3, one 81-week-old and two 86-week-old), 40-45 g (N=6, four 33-34-week-old, two 64-week-old), 45-50 g (N=10, two 33-34-week-old, two 40-week-old, three 64-65-week-old, two 83-86-week-old, one 93-week-old), and >50 g (N=2, 86 and 93-week-old). Mice above 40 g in body weight did not have significantly different lipid percentages to each other, whereas mice with <40 g body weight had significantly lower lipid accumulation in PVAT (asterisk, p<0.01) (A). Representative images of PVAT are shown for three mice: one <40 g, one ~40 g, and one 53 g (B). Increases in whole depot ing-scWAT nerve area (TH) and vascular area (IB4) were found to correlate with increased tissue weight in male HET3 mice N=15. Normalizing TH and IB4 area to total tissue area corrected for this correlation (C). Correlation of relative nerve fiber density and vascular density (D). Related to data presented in FIG. 5D-F (C-D). Intact inguinal scWAT depots were excised from male and female HET3 mice at 62 weeks and 126 weeks (N=4-6) and whole mount stained for the pan-neuronal marker PGP9.5 and the vascular marker IB4 (E). Representative images were captured with 10× and 40× objectives on a Nikon Eclipse E400 epifluorescence microscope (E). Scale bars are 50 μm (B), 200 μm and 50 μm (E). One-way ANOVA with Tukey's correction for multiple comparisons (C). Linear regression goodness of fit measured by R-squared and the significance of slope determined by F-test (C-D). Error bars are SEMs. p>0.05, *p<0.05, **p<0.01, ***p<0.001, ****p<0.0001.

FIG. 13 (A-C) shows the number of neuro-adipose nexuses (NANs) per ing-scWAT depot does not correlate with tissue size, nerve density, or vascular density. Lack of any correlation in the number of NANs in each whole ing-scWAT depot with weight (A), relative nerve density (B), or vascular density (C). Measured in male HET3 mice (N=15). All data related to FIG. 6C. Linear regression goodness of fit measured by R-squared and the significance of slope determined by F-test.

FIG. 14 shows rapamycin treatment was effective at inhibiting mTORC1. Protein expression of p70S6K and p-p70S6K from male HET3 mice in the late-intervention group (Veh N=4, Rapa N=3) normalized to the housekeeping protein cyclophilin b (A). Quantified as a ratio of normalized protein expression. Unpaired two-tailed Student's t-test. Error bars are SEMs. p>0.05, *p<0.05, **p<0.01, ***p<0.001, ****p<0.0001.

FIG. 15 shows protein amino acid sequence for PSD95 has very high similarity (99.6%) between human and mouse PSD-95. Sequences are SEQ ID NOS: 51, 52, 43, and 54 from top to bottom.

FIG. 16 shows Neuro-Adipose Nexuses (NANs) were discovered in white adipose tissue and are marked by tyrosine hydroxylase (TH) and CGRP. Immunofluorescent imaging of NANs in scWAT with markers of sympathetic (TH) and sensory (CGRP) innervation, captured on confocal at 63×. . . . These NANs can envelop, or wrap around, individual adipocytes, and are aputative nerve ending. These structures are rare and can be plastic, or remodel frequently in response to stimuli or tissue needs. These structures can occur at the end of axons that present with varicosities, indicating en passant release of nerve products (neurotransmitters, neuropeptides) and the axons express presynaptic markers (synapsin) and vesicle markers (SV2).

FIG. 17 (A-B) shows that a high fat, high sugar (HFD) neuropathic diet resulted in paw skin neuropathy and increased expression of PSD-95. (A) shows total tissue innervation as assessed by protein expression of PGP 9.5, a pan-neuronal marker. (B) shows protein expression of PSD-95, a post-synaptic density marker. Western blot data normalized to indicated housekeepers, band intensities quantified in Image J, and analyzed by two-tailed Student's t-test. Error bars are SEMs, as measured by western blotting.

FIG. 18 (A-B) shows HFD-induced neuropathy extends into scWAT and is accompanied by a trend for increased PSD95, likely indicating pathological nerve sprouting as occurs early in peripheral neuropathy (PN). (A) Protein expression of PGP 9.5 and (B) PSD9.5 in mouse scWAT. Western blot data normalized to indicated housekeepers, band intensities quantified in Image J, and analyzed by two-tailed Student's t-test. Error bars are SEMs.

FIG. 19 (A-B) shows reversal of neuropathy via pharmacological MMP-13 inhibition increased PGP9.5 expression, indicative of axon outgrowth and nerve regeneration. (A) Protein expression of PGP 9.5 and (B) PSD9.5 in mouse paw skin from HFD neuropathic animals with (HFD+Drug) or without (HFD+Vehicle) MMP13 inhibitor treatment. Western blot data normalized to indicated housekeepers, band intensities quantified in Image J, and analyzed by two-tailed Student's t-test. Error bars are SEMs.

FIG. 20 shows Rec2-AAV-BDNF delivery to mouse scWAT boosted PSD-95 expression in diet-induced neuropathic mice—also indicative of regeneration. Western blot data normalized to indicated housekeepers, band intensities quantified in Image J, and analyzed by two-tailed Student's t-test. Error bars are SEMs.

FIG. 21 (A-B) shows PSD-95 was not affected by cold-induced neuroplasticity but was more prevalent in BAT (which has higher TH/SNS activity and may not require axon branching/outgrowth). (A) shows PSP95 protein expression in male and female scWAT and BAT tissue. (B) shows PSD95 protein expression does not change in scWAT between room temperature, thermoneutral of 3 days cold exposed animals. Western blot data normalized to indicated housekeepers, band intensities quantified in Image J, and analyzed by two-tailed Student's t-test. Error bars are SEMs.

FIG. 22 (A-B) shows a reduction of adipose PSD-95 expression correlated with neuropathy (reduced TH) in a severe, genetically obesity/diabetes mouse model. (A) Protein expression of tyrosine hydroxylase (TH) sympathetic nerve marker, and (B) PSD95 in scWAT of lean BTBR WT mice, obese 12 wks old BTBR MUT mice and obese 24 wks old BTBR MUT mice. Western blot data normalized to indicated housekeepers, band intensities quantified in Image J, and analyzed by two-tailed Student's t-test. Error bars are SEMs.

FIG. 23 shows PSD-95 was increased (gene expression) in obese human subcutaneous adipose.

FIG. 24A-C shows that daily intra-adipose administration of CL316 243, a highly selective beta3-adrenergic receptor agonist (mimics SNS nerve activity) for 7 days, leads to (A) increased PSD95 expression and an upward trend in (B) PGP9.5 Protein expression in scWAT. Western blot data normalized to indicated housekeepers, band intensities quantified in Image J, and analyzed by two-tailed Student's t-test. Error bars are SEMs.

FIG. 25 (A-B) shows that (A) recombinant anti-PSD95 antibody from Abcam (cat #ab238135) does not effectively detect PSD95 in mouse scWAT, but (B) anti-PSD95 antibody from Abcam (cat #ab18258) does.

FIG. 27 (A-B) shows a comparison of two different antibodies. Both (A) Abcam #18258 antibody and (B) Cell signaling #3450 antibody can detect PSD95 in mouse tissue lysates.

FIG. 28 shows neuroMab PSD95 antibody Cat #75-028 can detect PSD95 in mouse brain and scWAT.

### DETAILED DESCRIPTION

### Definitions

In this specification and in the claims which follow, reference will be made to a number of terms which shall be defined to have the following meanings:

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that throughout the application, data is provided in a number of different formats, and that this data represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise.

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “antibody” is used in the broadest sense, and specifically covers monoclonal antibodies (including full length monoclonal antibodies), polyclonal antibodies, and multispecific antibodies (e.g., bispecific antibodies). Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrametric glycoproteins of about 150,000 Daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

As used herein, the term “buffer” refers to a solution consisting of a mixture of acid and its conjugate base, or vice versa. The solution is used as a means of keeping the pH at a nearly constant range to be used in a wide variety of chemical and biological applications.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

As used herein, “diagnose,” “diagnosed,” “diagnosing,” and any grammatical variations thereof as used herein, refers to the act of process of identifying the nature of an illness, disease, disorder, or condition in a subject by examination or monitoring of symptoms.

The term “detect” or “detecting” refers to an output signal released for the purpose of sensing of physical phenomenon. An event or change in environment is sensed and signal output released in the form of light.

“Differentially expressed” as applied to a gene or protein, refers to the differential production of the mRNA transcribed from the gene, or the protein product. A differentially expressed gene (or its protein product) may be overexpressed or under expressed as compared to the expression level of a normal or control cell. In one aspect, it refers to a differential that is 2.5 times, preferably 5 times, or preferably 10 times higher or lower than the expression level detected in a control sample. The term “differentially expressed” also refers to nucleotide sequences or proteins in a cell or tissue which are expressed/present where silent in a control cell or not expressed/present were expressed in a control cell.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. For example, a decrease can mean in protein expression, such as of PSD-95. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Expression” as used herein refers to the process by which information from a gene is used in the synthesis of a functional gene product that enables it to produce a peptide/protein end product, and ultimately affect a phenotype, as the final effect.

A “gene” refers to a polynucleotide containing at least one open reading frame that is capable of encoding a particular polypeptide or protein after being transcribed and translated. Any of the polynucleotide's sequences described herein may be used to identify larger fragments or full-length coding sequences of the gene with which they are associated. Methods of isolating larger fragment sequences are known to those of skill in the art, some of which (DLG4) are described herein.

An “increase” can refer to any change that results in a greater amount of a symptom, disease, composition, condition, or activity. An increase can be any individual, median, or average increase in a condition, symptom, activity, composition in a statistically significant amount. For example, a decrease can mean in protein expression, such as of PSD-95. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the term “lysis” refers to the process of breaking down the membrane of a cell, often by viral, enzymatic, or osmotic mechanisms that compromise cellular integrity.

A “neurodegenerative disease” is caused by the progressive loss of structure or function of neurons or glial cells, which make up the nervous system. These diseases include but are not limited to Peripheral Neuropathy, Amyotrophic Lateral Sclerosis (ALS), multiple sclerosis, Parkinson's disease, Alzheimer's disease, Huntington's disease, and prion diseases. Neurodegenerative diseases can lead to cognitive and physical impairments, neuroinflammation (inflammation of the brain and spinal cord), and deterioration of brain and spinal cord tissues.

A “nucleic acid” is a chemical compound that serves as the primary information-carrying molecules in cells and makes up the cellular genetic material. Nucleic acids comprise nucleotides, which are monomers made of a 5-carbon sugar (usually ribose or deoxyribose), a phosphate group, and a nitrogenous base. A nucleic acid can also be a deoxyribonucleic acid (DNA) or a ribonucleic acid (RNA). A chimeric nucleic acid comprises two or more of the same kind of nucleic acid fused together to form one compound comprising genetic material.

As used herein, the term “Polymerase Chain Reaction” (“PCR”) refers to a method for increasing the concentration of a segment of a target sequence in a mixture of genomic DNA without cloning or purification. This process for amplifying the target sequence typically consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured, and the primers then annealed to their complementary sequences within the target molecule. Following annealing, the primers are extended with a polymerase so as to form a new pair of complementary strands. The steps of denaturation, primer annealing, and polymerase extension can be repeated many times to obtain a high concentration of an amplified segment of the desired target sequence. Unless otherwise noted, PCR, as used herein, also includes variants of PCR such as allele-specific PCR, asymmetric PCR, hot-start PCR, ligation-mediated PCR, multi-plex-PCR, reverse transcription PCR, or any of the other PCR variants known to those skilled in the art.

As used herein, the term “polymerase” refers to an enzyme that synthesizes long chains of polymers or nucleic acids. DNA polymerase and RNA polymerase are used to assemble DNA and RNA molecules, respectively, by copying a DNA template strand using base-pairing interactions.

A “protein,” “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

A “primer” is a short polynucleotide, generally with a free 3′—OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in “PCR: A PRACTICAL APPROACH” (M. MacPherson et al., IRL Press at Oxford University Press (1991)). All processes of producing replicate copies of a polynucleotide, such as PCR or gene cloning, are collectively referred to herein as “replication.” A primer can also be used as a probe in hybridization reactions, such as Southern or Northern blot analyses. Sambrook et al., supra.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes. As used herein, the term “probe” can also refer to an antibody, a molecule or group of molecules used in molecular biology or chemistry to study the properties of other molecules or structures. If some measurable property of the molecular probe used changes when it interacts with the molecule of interest, the interactions between the probe and the molecule of interest can be studied. This makes it possible to indirectly study the properties of compounds and structures which may be hard to study directly.

By “reduce” or other forms of the word, such as “reducing” or “reduction,” is meant lowering of an event or characteristic (e.g., PSD-95 levels or symptoms of peripheral neuropathy). It is understood that this is typically in relation to some standard or expected value, in other words it is relative, but that it is not always necessary for the standard or relative value to be referred to. For example, “reduces PSD-95 levels” means reducing the level of PSD-95 compared to a standard or a control.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “treat,” “treating,” and grammatical variations thereof as used herein, include partially or completely delaying, alleviating, mitigating, or reducing the intensity of one or more attendant symptoms of a disorder or condition and/or alleviating, mitigating, or impeding one or more causes of a disorder or condition. Treatments according to the disclosure may be applied preventively, prophylactically, palliatively, or remedially. Treatments are administered to a subject prior to onset (e.g., before obvious signs of peripheral neuropathy), during early onset (e.g., upon initial signs and symptoms of peripheral neuropathy), or after an established development of peripheral neuropathy.

A “receptor” is a cellular protein whose activation causes a cell to modify its present functions or actions.

“Marker” in the context of the present invention refers to a polypeptide (of a particular apparent molecular weight) which is differentially present in a sample taken from patients having peripheral neuropathy as compared to a comparable sample taken from control subjects (e.g., a person with a negative diagnosis, normal or healthy subject).

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of neural injury and/or neuronal disorder. A diagnostic amount can be either in absolute amount (e.g., μg/ml) or a relative amount (e.g., relative intensity of signals).

A “control” is an alternative subject or sample used in an experiment for comparison purposes. A control can be “positive” or “negative.”

A “control amount” of a marker can be any amount or a range of amount which is to be compared against a test amount of a marker. For example, a control amount of a marker can be the amount of a marker in a person without peripheral neuropathy. A control amount can be either in absolute amount (e.g., μg/ml) or a relative amount (e.g., relative intensity of signals).

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.”

“Adsorption” or “retention” refers to the detectable binding between an absorbent and a marker either before or after washing with an eluant (selectivity threshold modifier) or a washing solution.

“Eluant” or “washing solution” refers to an agent that can be used to mediate adsorption of a marker to an adsorbent. Eluants and washing solutions are also referred to as “selectivity threshold modifiers.” Eluants and washing solutions can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

“Detectable moiety” or a “label” refers to a composition detectable by spectroscopic, photochemical, biochemical, immunochemical, or chemical means. For example, useful labels include 32P, 35S, fluorescent dyes, electron-dense reagents, enzymes (e.g., as commonly used in an ELISA), biotin-streptavidin, digoxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, or flow cytometry.

“Immunoassay” is an assay that uses an antibody to specifically bind an antigen (e.g., a marker). The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker PSD-95 from specific species such as rat, mouse, or human can be selected to obtain only those polyclonal antibodies that are specifically immunoreactive with marker PSD-95 and not with other proteins, except for polymorphic variants and alleles of marker PSD-95. This selection may be achieved by subtracting out antibodies that cross-react with marker PSD-95 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically, a specific or selective reaction will be at least twice background signal or noise and more typically more than 10 to 100 times background.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Substantially purified” refers to nucleic acid molecules or proteins that are removed from their natural environment and are isolated or separated, and are at least about 60% free, preferably about 75% free, and most preferably about 90% free, from other components with which they are naturally associated.

“Substrate” refers to any rigid or semi-rigid support to which nucleic acid molecules or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels, and pores.

“Neural cells” as defined herein, are cells that reside in the brain, central and peripheral nerve systems, including, but not limited to, nerve cells, glial cell, oligodendrocyte, microglia cells or neural stem cells.

“Neuronal specific or neuronally enriched proteins” are defined herein, as proteins that are present in neural cells and not in non-neuronal cells, such as, for example, cardiomyocytes, myocytes, in skeletal muscles, hepatocytes, kidney cells and cells in testis.

“Neural (neuronal) defects, disorders or diseases” as used herein refers to any neurological disorder, including but not limited to neurodegenerative disorders (Parkinson's; Alzheimer's) or autoimmune disorders (multiple sclerosis) of the central nervous system; memory loss; long term and short term memory disorders; learning disorders; autism, depression, benign forgetfulness, childhood learning disorders, close head injury, and attention deficit disorder; autoimmune disorders of the brain, neuronal reaction to viral infection; brain damage; depression; psychiatric disorders such as bi-polarism, schizophrenia and the like; narcolepsy/sleep disorders (including circadian rhythm disorders, insomnia and narcolepsy); severance of nerves or nerve damage; severance of the cerebrospinal nerve cord (CNS) and any damage to brain or nerve cells; neurological deficits associated with AIDS; tics (e.g. Giles de la Tourette's syndrome); Huntington's chorea, schizophrenia, traumatic brain injury, tinnitus, neuralgia, especially trigeminal neuralgia, neuropathic pain, inappropriate neuronal activity resulting in neurodysthesias in diseases such as diabetes, MS and motor neuron disease, ataxias, muscular rigidity (spasticity) and temporomandibular joint dysfunction; Reward Deficiency Syndrome (RDS) behaviors in a subject.

### General Description

The present invention identifies biomarkers that are diagnostic of peripheral neuropathy. Detection of different biomarkers of the invention are also diagnostic of the degree of severity of peripheral neuropathy. The phrase “differentially present” refers to differences in the quantity and/or the frequency of a marker present in a sample taken from patients having, for example, neural injury as compared to a control subject. For example, a marker can be a polypeptide which is present at an elevated level or at a decreased level in samples of patients with peripheral neuropathy compared to samples of control subjects. Alternatively, a marker can be a polypeptide which is detected at a higher frequency or at a lower frequency in samples of patients compared to samples of control subjects. A marker can be differentially present in terms of quantity, frequency, or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively, or additionally, a polypeptide is differentially present between the two sets of samples if the frequency of detecting the polypeptide in samples of patients' suffering from peripheral neuropathy, is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently than the control sample. Alternatively, a polypeptide is differentially present between the two sets of samples if it is detected at less than 90%, less than 80%, less than 70%, less than 60%, less than 50%, less than 40%, less than 30%, less than 20%, less than 10%, or at 5% or less than the control sample.

PSD-95, a scaffolding protein, is located in the membranes of excitatory neurons at the post synaptic ends wherein, neurons or nerve cells communicate through action potentials, which are either excitatory or inhibitory. Action potentials are electrical impulses that send signals throughout the body and are a temporary shift from a negative state to a positive state in the neuron's membrane potential caused by negative or positive ions flowing in and out of the neuron. In some embodiments, action potential can be excitatory currents wherein the excitatory currents are those that prompt one neuron to share information with the next neuron. The presynaptic terminal is at the end of an axon and is the place where the electrical signal (the action potential) is converted into a chemical signal (neurotransmitter release). The postsynaptic terminal membrane is less than 50 nanometers away and contains specialized receptors. The binding of neurotransmitters, either directly or indirectly, to the specialized receptors on the postsynaptic terminal membrane causes ion channels in the postsynaptic membrane to open or close resulting in ion fluxes. The ion fluxes thereby change the membrane potential of the postsynaptic cell, thus mediating the transfer of information across the synapse. PSD-95 is a major regulator of synaptic maturation by interacting, stabilizing, and trafficking N-methyl-D-aspartic acid receptors (NMDARs) and α-amino-3-hydroxy-5-methyl-4-isox-azoleproprionic acid receptors (AMPARs) to the postsynaptic membrane. NMDAR and AMPAR are ion channels allowing action potentiation. PSD-95 correlates with synaptic density.

By synaptic density is meant the net number of surviving synapses. It is noted that this number changes very little in adulthood, except due to the influence of neurodevelopmental abnormalities, or during some neurodegenerative disorders like peripheral neuropathy. PSD-95 is a post-synaptic protein, and its expression varies according to tissue innervation/neuropathy state. PSD-95 can therefore be used as a marker of aging, obesity, or other peripheral neuropathy-based associations.

The kit disclosed herein comprises a means for detecting PSD-95. Particularly disclosed herein is detection of the marker PSD-95 as a marker of peripheral neuropathy, kits for detecting PSD-95, and methods of treatment of peripheral neuropathy based on the detection of PSD-95. Other biomarkers can also be detected along with PSD-95, and these additional biomarkers can also be used to determine a method of treatment for a subject in need. These additional biomarkers include, but are not limited to, PGP9.5, CGRP, GAP43 and tyrosine hydroxyl.

### Methods of Diagnosing Peripheral Neuropathy

Detection of at least PSD-95 in adipose samples, interstitial fluid, blood, or other biological fluids, can be diagnostic of the severity of peripheral neuropathy. It can also be used as a means to predict future peripheral neuropathy in a subject. These findings can then be correlated with various methods of treating the subject, which are discussed in more detail below. It is noted that by determining presence, or levels of, PSD-95, the subject can be then treated in a specific manner. Importantly, without PSD-95 measurements, accurate dosing, regimens, and treatment modalities would not have been attained. In other words, treatment types and modalities are explicitly informed by the levels of PSD-95 detected in a subject. Without those markers, precise methods of treatment would not be available.

In one embodiment, the invention provides for the quantitative detection of peripheral neuropathy by detection of PSD-95 protein or nucleic acids encoding PSD-95. For example, quantified detection of PSD-95 can be used to assess the severity or type of peripheral neuropathy. Quantitation of PSD-95 can be done by nucleic acid detection (such as qPCR), immunoassays (such as ELISA's), spectrophotometry, HPLC, SELDI, or biochips, for example. Alternatively, nucleic acid encoding PSD-95 can be detected. Genetic analysis of PSD-95, optionally along with other biomarkers, can be used to calculate a risk score of having or developing peripheral neuropathy.

This risk score can comprise cutoff values, which can be used to calculate a score. The score can then be used to determine whether the subject has, or is at risk of developing, peripheral neuropathy, or to determine a classification or “level” of peripheral neuropathy.

Optionally, the detecting step comprises detecting mRNA levels of the biomarker. The mRNA detection can, for example, comprise reverse-transcription polymerase chain reaction (RT-PCR), quantitative real-time PCR (qRT-PCR), Northern analysis, microarray analysis, and cDNA-mediated annealing, selection, extension, and ligation (DASL) assay (Illumina, Inc.; San Diego, Calif.). Preferably, the RNA detection comprises the cDNA-mediated annealing, selection, extension, and ligation (DASL) assay (Illumina, Inc.). Optionally, the detecting step comprises detecting miRNA levels of the biomarker. The miRNA detection can, for example, comprise miRNA chip analysis, Northern analysis, RNase protection assay, in situ hybridization, miRNA expression profiling panels (Illumina, Inc.), or a modified reverse transcription quantitative real-time polymerase chain reaction assay (qRT-PCR). Preferably the miRNA detection comprises the miRNA expression profiling panels (Illumina, Inc.). Optionally, the detecting step comprises detecting mRNA and miRNA levels of the biomarker(s). The analytical techniques used to determine mRNA and miRNA expression are known. See, e.g., Sambrook et al., Molecular Cloning: A Laboratory Manual, 3rd Ed., Cold Spring Harbor Press, Cold Spring Harbor, N.Y. (2001), Yin et al., Trends Biotechnol. 26:70-6 (2008); Wang and Cheng, Methods Mol. Biol. 414:183-90 (2008); Einat, Methods Mol. Biol. 342:139-57 (2006).

Optionally, the detecting step comprises detecting the protein expression levels of the biomarkers. The protein detection can, for example, comprise an assay selected from the group consisting of Western blot, enzyme-linked immunosorbent assay (ELISA), enzyme immunoassay (EIA), radioimmunoassay (RIA), immunohistochemistry, and protein array. The analytical techniques used to determine protein expression are known. See, e.g., Sambrook et al., Molecular Cloning: A Laboratory Manual, 3rd Ed., Cold Spring Harbor Press, Cold Spring Harbor, N.Y. (2001).

The control can be a standardized gene panel obtained from one or more subjects who had not been diagnosed with peripheral neuropathy when or before control sampling took place. The standardized gene panel obtained from one or more control subjects can be obtained from a sample. The control used to compare the genetic marker panel can be obtained from one or more subjects which have been assessed and found not to have peripheral neuropathy, or which have been assessed and found to have peripheral neuropathy.

The control can also be from the subject being analyzed themselves and can be from a different time point. For example, the control can be from an earlier time period when the subject was not aged, or when the subject had not been diagnosed with peripheral neuropathy, or at an earlier timepoint. In this way, the control can serve as a measure of increasing peripheral neuropathy as a function of time in the subject. The control can be used to establish marker levels at different dosages or with various other treatments. One of skill in the art will understand how a control can be used, and varied, to inform a skilled artisan of how a subject is responding to treatment over time, or how a subject's disease or disorder is progressing (or regressing) over time.

According to the present invention, a “baseline” or “control” can include a normal or negative control and/or a disease or positive control, against which a test level of PSD-95 can be compared. Therefore, it can be determined, based on the control or baseline level of PSD-95, whether a sample to be evaluated for peripheral neuropathy has a measurable difference or substantially no difference in PSD-95 levels, as compared to the baseline level. In one aspect, the baseline control is indicative of the level of PSD-95 as expected in a normal (e.g., healthy, negative control) patient. Therefore, the term “negative control” used in reference to a baseline level of PSD-95 typically refers to a baseline level of expression from a population of individuals which is believed to be normal (i.e., not having or developing peripheral neuropathy). In some embodiments of the invention, it may also be useful to compare the gene expression in a test sample to a baseline that has previously been established from a patient or population of patients with peripheral neuropathy. Such a baseline level, also referred to herein as a “positive control”, refers to a level of PSD-95 established in a sample from one or preferably a population of individuals who had been positively diagnosed with peripheral neuropathy.

In one embodiment, when the goal is to monitor the progression or regression of peripheral neuropathy in a patient, for example, to monitor the efficacy of treatment of the disease or to determine whether a patient that appears to be predisposed to the disease begins to develop the disease, one baseline control can include the measurements of PSD-95 in a sample from the patient that was taken from a prior test in the same patient. In this embodiment, a new sample is evaluated periodically (e.g., at annual or more regular physicals), and any changes in levels in the patient as compared to the prior measurement and most typically, also with reference to the above-described normal and/or positive controls, are monitored. Monitoring of a patient's PSD-95 levels can be used by the clinician to prescribe or modify treatment for the patient based on whether any differences in gene expression in the disease or disorder is indicated.

In one example, the control or baseline levels of PSD-95 levels are collected from “matched individuals”. According to the present invention, the phrase “matched individuals” refers to a matching of the control individuals on the basis of one or more characteristics, such as gender, age, race, or any relevant biological or sociological factor that may affect the baseline of the control individuals and the patient (e.g., preexisting conditions, consumption of particular substances, levels of other biological or physiological factors). The number of matched individuals from whom control samples must be obtained to establish a suitable control level (e.g., a population) can be determined by those of skill in the art but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values. It will be appreciated by those of skill in the art that a baseline need not be established for each assay as the assay is performed but rather, a baseline can be established by referring to a form of stored information regarding a previously determined control level of PSD-95. Such a form of stored information can include, for example, but is not limited to, a reference chart, listing or electronic file of population or individual data regarding “normal” (negative control) or positive PSD-95 levels; a medical chart for the patient recording data from previous evaluations; or any other source of data regarding control PSD-95 that is useful for the patient to be diagnosed or evaluated.

In comparison to currently existing products, the invention provides several superior advantages and benefits. First, the identification of PSD-95 provides more rapid and less expensive diagnosis of injury severity than existing diagnostic devices such as computed tomography (CT) and magnetic resonance imaging (MRI). The invention also allows quantitative detection and high content assessment of peripheral neuropathy. In addition, levels of PSD-95 provide more accurate information regarding the level of peripheral neuropathy than what is currently on the market.

A biological sample can be obtained from a subject by conventional techniques. Blood can be obtained by venipuncture, while plasma and serum can be obtained by fractionating whole blood according to known methods. Surgical techniques for obtaining solid tissue samples are well known in the art. For example, methods for obtaining a nervous system tissue sample are described in standard neurosurgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999. Methods for obtaining and analyzing brain tissue are also described in Belay et al., Arch. Neurol. 58:1673-1678 (2001); and Seijo et al., J. Clin. Microbiol. 38:3892-3895 (2000).

Any subject that expresses PSD-95 can be used as a subject from which a biological sample is obtained. Preferably, the subject is a mammal, such as for example, a human, dog, cat, horse, cow, pig, sheep, goat, primate, rat, mouse, and other vertebrates such as fish, birds, and reptiles. More preferably, the subject is a human. Particularly preferred are subjects suspected of having or at risk for developing peripheral neuropathy, such as those who are aging. Examples of “aging” include subjects at or over the age of 40, 45, 50, 55, 60, 65, 70, 75, 80, or older.

The biomarkers of the invention can be detected in a sample by any means. Methods for detecting the biomarkers are described in detail in the materials and methods and Examples which follow. For example, as mentioned above, immunoassays, include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, fluorescent immunoassays, and the like. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding an antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen (i.e. neural biomarker), coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

A variety of methods exist in the art for the production of monoclonal antibodies and thus, the invention is not limited to their sole production in hybridomas. For example, the monoclonal antibodies may be made by recombinant DNA methods, such as those described in U.S. Pat. No. 4,816,567. In this context, the term “monoclonal antibody” refers to an antibody derived from a single eukaryotic, phage, or prokaryotic clone. The DNA encoding the monoclonal antibodies of the invention can be readily isolated and sequenced using conventional procedures (e.g., by using oligonucleotide probes that are capable of binding specifically to genes encoding the heavy and light chains of murine antibodies, or such chains from human, humanized, or other sources). The hybridoma cells of the invention serve as a preferred source of such DNA. Once isolated, the DNA may be placed into expression vectors, which are then transformed into host cells such as Simian COS cells, Chinese hamster ovary (CHO) cells, or myeloma cells that do not otherwise produce immunoglobulin protein, to obtain the synthesis of monoclonal antibodies in the recombinant host cells.

Methods for producing and screening for specific antibodies using hybridoma technology are routine and well known in the art. In a non-limiting example, mice can be immunized with a biomarker polypeptide or a cell expressing such peptide. Once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind PSD-95.

Other methods can also be used for the large-scale production of PSD-95 specific antibodies. For example, antibodies can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human, or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene III or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187 9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

The antibodies of the present invention have various utilities. For example, such antibodies may be used in diagnostic assays to detect the presence or quantification of the polypeptides of the invention in a sample. Such a diagnostic assay can comprise at least two steps. The first, subjecting a sample with the antibody, wherein the sample is a tissue (e.g., human, animal, etc.), biological fluid (e.g., blood, urine, sputum, semen, amniotic fluid, saliva, etc.), biological extract (e.g., tissue or cellular homogenate, etc.), a protein microchip (e.g., See Arenkov P, et al., Anal Biochem., 278 (2): 123-131 (2000)), or a chromatography column, etc. And a second step involving the quantification of antibody bound to the substrate. Alternatively, the method may additionally involve a first step of attaching the antibody, either covalently, electrostatically, or reversibly, to a solid support, and a second step of subjecting the bound antibody to the sample, as defined above and elsewhere herein.

As mentioned above, the antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 125I, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem., 13:1014 (1974); Pain et al., J. Immunol. Methods, 40: 219 (1981); and Nygren, J. Histochem, and Cytochem., 30:407 (1982).

### Methods of Treating Peripheral Neuropathy

Disclosed herein is a method of treating or preventing neuropathy in a subject. The method comprises detecting PSD-95 levels in a sample from the subject, wherein, when the PSD-95 level differs from a control, the subject is treated for neuropathy. The control, shown herein, is a measurement at a different timepoint from the subject themselves and is based on PSD-95 level in a subject not experiencing neuropathy. In some embodiments, the PSD-95 is decreased by 10%, 20%, 30%, 40%, 50%, 60%, 70%, 80%, 90% or 100% compared to control. As disclosed herein, PSD-95 is detected using the kit disclosed herein. In some embodiments, the sample is derived from adipose samples or interstitial fluid, or other bodily fluid samples. In some embodiments, samples are from skin or subdermal using the circular blade provided in the kit.

The method of treatment can comprise modulating adipose number and/or size by using a therapy selected from a group comprising calorie-restricted diets/exercise, cholesterol-free diets, low-fat diets, the use of bioactive compounds, pharmacological compounds, or a combination thereof. In some embodiments, the method treating or preventing symptoms in a subject comprises administering a PSD-95 modulator to the subject. In some embodiments, the subject is treated with a pharmacological MMP-13 inhibitor.

After treatment, the effectiveness of treatment can be assessed by measuring PSD-95 levels using the kit disclosed herein. In some embodiments, the effectiveness of the treatment is validated by immunostaining to measure PSD-95 levels in one target selected from the group comprising nerve fiber density, Intradermal N, number of terminal Schwann cells in a neuromuscular junction, adiposity, adipose cell size, adipose tissue inflammation.

### Kits

Disclosed herein is a kit for determining an amount of post-synaptic density protein-95 (PSD-95) present in a subject.

In yet another aspect, the invention provides kits for aiding a diagnosis of peripheral neuropathy, or degree of severity of peripheral neuropathy, wherein the kits can be used to detect PSD-95. For example, the kits can be used to detect any one or more of the markers described herein, which markers are differentially present in samples of a patient and normal subjects. The kits of the invention have many applications. For example, the kits can be used to identify compounds that modulate expression of one or more of the markers in in vitro or in vivo animal models. This can be used to identify new compounds for treating or preventing peripheral neuropathy, or to determine the effects of treatment of a known compound. It can also be used to diagnose, or determine the severity of, peripheral neuropathy in a subject. Furthermore, it can be used to determine that a subject is likely to experience symptoms of peripheral neuropathy if they are not already.

In one embodiment, a kit comprises (a) an antibody that specifically binds to a marker; and (b) a detection reagent. Such kits can be prepared from the materials described above, and the previous discussion regarding the materials (e.g., antibodies, detection reagents, immobilized supports, etc.) is fully applicable to this section and will not be repeated. Optionally, the kit may further comprise pre-fractionation spin columns. In some embodiments, the kit may further comprise instructions for suitable operation parameters in the form of a label or a separate insert.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate, or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Optionally, the kit may further comprise a standard or control information so that the test sample can be compared with the control information standard to determine if the test amount of a marker detected in a sample is a diagnostic amount consistent with a diagnosis of peripheral neuropathy and/or effect of treatment on the patient.

In another embodiment, a kit comprises: (a) a substrate comprising an adsorbent thereon, wherein the adsorbent is suitable for binding a marker, and (b) instructions to detect PSD-95 by contacting a sample with the adsorbent and detecting the marker or markers retained by the adsorbent. In some embodiments, the kit may comprise an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In another embodiment, the kit may comprise a first substrate comprising an adsorbent thereon (e.g., a particle functionalized with an adsorbent) and a second substrate onto which the first substrate can be positioned to form a probe which is removably insertable into a gas phase ion spectrometer. In other embodiments, the kit may comprise a single substrate which is in the form of a removably insertable probe with adsorbents on the substrate. In yet another embodiment, the kit may further comprise a pre-fractionation spin column (e.g., Cibacron blue agarose column, anti-HSA agarose column, size exclusion column, Q-anion exchange spin column, single stranded DNA column, lectin column, etc.).

Optionally, the kit can further comprise instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

The kit can also be used to detect nucleic acid encoding PSD-95. Suitable nucleic acid samples for screening on an array contain transcripts of interest or nucleic acids derived from the transcripts of interest (i.e., transcripts derived from the genes associated with diseases and disorders of the present invention). As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like. Preferably, such a sample is a total RNA preparation of a biological sample (e.g., peripheral blood mononuclear cells or PBMCs). More preferably in some embodiments, such a nucleic acid sample is the total mRNA isolated from such a biological sample.

In specific examples, the kit disclosed herein can comprise the means for detecting PSD-95. The means can be a probe and in some embodiments the probe can be an antibody which detects a protein, such as an Enzyme-linked Immunosorbent Assay (ELISA). Apart from the probe, the kit can further comprise a testing well, a secondary fluorescent detection agent, a wash buffer or extraction buffer, a signal amplification buffer, or a combination thereof. In some embodiments, the extraction buffer can be a lysis buffer that causes the sample tissue to break down and release proteins and nucleic acids for testing. In some embodiments, the probe can be a nucleic acid, for example, a set of primers that can detect a nucleic acid in a means that comprises a Polymerase Chain Reaction (PCR) assay. The PCR kit can comprise at least one of a polymerase enzyme, nuclease-free water, MgCl₂, deoxynucleotide triphosphates (dNTPs), or a combination thereof. The testing well can be a 96-well plate which has the probe coated onto the base of a testing well. The kit can further comprise a circular blade for sample collection prior to the use of the kit or any additional means for detecting any additional marker. In some embodiments, the additional marker comprises one selected from a group comprising PGP9.5, CGRP, GAP43 or tyrosine hydroxyl (TH), wherein PGP9.5 is a pan neuronal marker used in clinical histological assessment of Peripheral neuropathy in skin biopsy punches. CGRP is a sensory neuropeptide marking sensory axons. GAP43 is an axon outgrowth/growth cone marker and TH is a sympathetic marker wherein sympathetic is that branch of the nervous system that responds in dangerous and stressful situations.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### Example 1: Age-Related Changes to Adipose Tissue and Peripheral Neuropathy Differ By Mouse Sex and Genetic Background and are not Mitigated by Rapamycin Longevity Treatment

### Abstract

Neural communication between the brain and adipose tissues regulates energy expenditure and metabolism through modulation of adipose tissue functions. It has recently been demonstrated that under pathophysiological conditions (obesity, diabetes, and aging), total subcutaneous white adipose tissue (scWAT) innervation is decreased (‘adipose neuropathy’). With advanced age in the C57BL/6J mouse, small fiber peripheral nerve endings in adipose tissue die back, resulting in reduced contact with adipose-resident blood vessels and other cells. This vascular neuropathy and parenchymal neuropathy together likely pose a physiological challenge for tissue function. In the current work, the genetically diverse HET3 mouse model was used to investigate the incidence of peripheral neuropathy and adipose tissue dysregulation across several ages in both male and female mice. The anti-aging treatment rapamycin, an mTOR inhibitor, was also investigated as a means to prevent or reduce adipose neuropathy. It was found that HET3 mice displayed a reduced neuropathy phenotype compared to inbred C56BL/6J mice, indicating genetic contributions to this aging phenotype. Compared to female HET3 mice, male HET3 mice had worse neuropathic phenotypes by 62 weeks of age. Female HET3 mice appeared to have increased protection from neuropathy until advanced age (126 weeks), after reproductive senescence. It was found that rapamycin overall had little impact on neuropathy measures, and actually worsened adipose tissue inflammation and fibrosis. Despite its success as a longevity treatment in mice, higher doses and longer delivery paradigms for rapamycin may lead to a disconnect between lifespan and beneficial health outcomes.

### Introduction

For this study male and female HET3 mice, the genetically diverse mouse strain utilized by the National Institute on Aging (NIA)'s Interventions Testing Program (ITP; (Miller et al., 2007)) were used. The HET3 mouse population is produced by crossing BALB/cJ×C57BL/6J F1 females mated to C3H/HeJ×DBA/2J F1 males. This four-way cross of inbred strains creates reproducible genetic variability in the offspring (Miller, Burke, & Nadon, 1999), and age-related mortality will therefore not be the result of a strain-specific disease (Flurkey, Astle, & Harrison, 2010), making HET3 a more relevant model for investigating human aging pathophysiology. Importantly, the average lifespan of HET3 mice is approximately 120 weeks, which is the same as the inbred C57BL/6J (BL6) strain used as a reference in this study (Flurkey et al., 2010; Yuan et al., 2012).

To date, the ITP has tested over 50 treatments to assess improvements to longevity in the HET3 mouse, across 3 testing sites (The Jackson Laboratory, The University of Michigan, and the University of Texas Health Science Center), and 5 of these treatments were moved to a second phase of testing due to effectiveness at extending mean lifespan; one of which was rapamycin. Rapamycin increased lifespan of both male and female HET3 mice, regardless of starting the treatment early (Miller et al., 2011) or late in life (Harrison et al., 2009). (Of note, the majority of other ITP-tested longevity candidate treatments were only effective in expanding lifespan in males, also underscoring the striking sex differences in aging processes.)

Rapamycin is an inhibitor of the mechanistic target of rapamycin (mTOR) protein which is an integral component in several pathways that govern cell survival, cell proliferation, lipid metabolism, lipid synthesis, and adipogenesis, among many others (Laplante & Sabatini, 2009). mTOR functions as the catalytic component of two protein complexes: rapamycin-sensitive mTORC1 and rapamycin-insensitive mTORC2 (Laplante & Sabatini, 2009). Acute rapamycin treatment inhibits mTORC1 promoting increased lifespan, but chronic (on the order of weeks to months) rapamycin treatment can inhibit mTORC2 in addition to its main target of mTORC1, leading to glucose intolerance (Lamming et al., 2013), delayed glucose clearance (Reifsnyder, Te, & Harrison, 2020), insulin insensitivity in BL6 (Lamming et al., 2012) but not HET3 mice (Lamming et al., 2013), hyperlipidemia, and reduced browning-potential of WAT (Tran et al., 2016). Interestingly, these are also all characteristics of advanced age (DeFronzo, 1981; Palmer & Kirkland, 2016; Rosada et al., 2020). A comprehensive set of experiments were carried out to see if rapamycin had any effect on several structural and functional aging phenotypes in BL6 mice (Neff et al., 2013), which revealed that while rapamycin did increase mean lifespan, only a handful of the aging phenotypes were improved, and these were also improved in young mice. This suggested that longevity effects were dissociated from effects on aging pathophysiology (Neff et al., 2013). Rapamycin has been shown to be ineffective at increasing the lifespan of some mouse strains with pre-existing metabolic disease, resulting in decreased mean lifespan (Selvarani, Mohammed, & Richardson, 2021). These include the obese and diabetic C57BL/KsJleprdb/db mice (Reifsnyder et al., 2018; Sataranatarajan et al., 2016), though there were clear cardioprotective effects in this strain after treatment (Reifsnyder et al., 2018).

Chronic rapamycin treatment has been shown to improve age-related learning and memory deficits in rodents (Neff et al., 2013) by attenuating the loss in synaptic density driven by mTOR (Van Skike et al., 2020). This poses an intriguing dichotomy in which chronic rapamycin-treatment can potentially restore nerve function centrally, while simultaneously not having any protective effects on peripheral nerve health, potentially indirectly by exacerbating diabetic status. Until now, this rapamycin longevity treatment had not yet been comprehensively investigated in adipose tissue function and peripheral neuropathy in the HET3 mouse.

For these reasons, the 42 ppm encapsulated rapamycin treatment in male and female HET3 mice, were utilized (previously shown to increase the lifespan of both sexes by 23-26% (Miller et al., 2014)) for a chronic 8-month intervention commenced at two different ages (creating early- and late-intervention groups) to investigate whether rapamycin treatment would attenuate or accelerate age-related neuropathy in scWAT and other peripheral tissues such as skin and muscle. Age-related impacts on adipose tissue health and peripheral neuropathy were not mitigated by rapamycin treatment, and adipose inflammation fibrosis were actually worsened after treatment, further underscoring the dissociation between lifespan and health outcomes of this longevity treatment.

### METHODS

### Animals

Female CByB6F1/J (JAX® #100009) bred to male C3D2F1/J (JAX® #100004) at the Jackson Laboratory to produce the male and female HET3 offspring used in this study. Male C57BL/6J (BL6) (JAX® #000664) were also bred and housed at the Jackson Laboratory in the same room as the HET3 mice to limit confounding variables. Mice were housed initially as 4 to a cage in a climate-controlled vivarium with 12/12 h light/dark cycle and ad libitum access to food and water. Mice were euthanized by CO2 asphyxiation with cervical dislocation as a secondary confirmation of death. All procedures were performed in compliance with the National Institute of Health Guide for the Care and Use of Laboratory Animals and were approved by an Institutional Animal Care and Use Committee.

### Rapamycin Treatment

Forty-two ppm rapamycin was microencapsulated in chow (LabDiet® 5LG6) and fed to male and female HET3 mice for 8 months prior to behavioral tests and tissue collection. Control mice not receiving rapamycin treatment received standard chow (LabDiet® 5LG6). All mice were fed ad libitum. Mice began rapamycin treatment at one of two ages. Mice that started rapamycin treatment at ~30 weeks of age served as the early-intervention group, and mice that started treatment at ~72 weeks of age served as the late-intervention group.

### Manual Von Frey Tactile Sensitivity and Nociception Test

Manual von Frey tactile sensitivity and nociception test was performed on BL6 and HET3 mice of various ages to determine tactile sensitivity of hind paw skin. Mice were acclimated on top of a grid platform in individual clear-walled compartments for 1 hr. Following acclimation period von Frey monofilaments were applied to mouse hind paw in order of decreasing strength (4.00 g, 2.00 g, 1.00 g, 0.40 g, and 0.02 g) (Stoelting, Cat #58011), repeated 5 times for each filament with a 5-minute interval between subsequent pokes on the same mouse. Each filament was applied to the mid-plantar surface of the hind paw and slight pressure applied until the mouse showed a response or the filament bent with force. A positive response was determined as immediate (<1 second) withdrawal or licking of the paw.

### Hematoxylin Staining and Cell Size Quantification

Intact inguinal scWAT (ing-scWAT), axillary scWAT (ax-scWAT), and perigonadal WAT (pgWAT) depots were excised from BL6 and HET3 mice, fixed in 10% buffered formalin overnight at room temperature and embedded in paraffin. Paraffin embedded tissues were sectioned 7 μm thick. Tissue sections were deparaffinized with HistoChoice Clearing Agent and hydrated in decreasing concentrations of EtOH (100%, 95% 70%, 30%, 0%). Tissues were stained in Mayer's Hematoxylin Solution (Sigma-Aldrich Cat #MHS16) or Mayer's Hemalum Solution (Sigma-Aldrich Cat #109249) and a drop of mounting fluid was applied, and tissues were cover slipped, sealed, and imaged. Three representative images were captured per tissue per animal and cell perimeter and area was measured in Fiji (Schindelin et al., 2012).

### Picrosirius Red Staining and Collagen Birefringence Quantification

Intact scWAT and pgWAT depots were excised from BL6 and HET3 mice, fixed in 10% buffered formalin overnight at room temperature and embedded in paraffin. Paraffin embedded tissues were sectioned 7 μm thick. Tissue sections were deparaffinized with HistoChoice Clearing Agent (Sigma-Aldrich Cat #H2779) and hydrated in decreasing concentrations of EtOH (100%, 95% 70%, 30%, 0%). Tissues were stained in picrosirius red stain (Electron Microscopy Sciences Cat #26357) for 1 hr, washed in two changes of acidified water (5 seconds each), dehydrated in three changes of 100% EtOH, cleared in histo-grade Xylenes for 5 minutes and cover slipped using Permount mounting media (Electron Microscopy Sciences Cat #17986-01). Five representative images per tissue were captured using both brightfield and circularly polarized light to gauge collagen distribution. Total collagen was measured by first quantifying the area comprised by picrosirius red staining in the field of view using brightfield microscopy. This was then divided by the area comprised exclusively of polarized collagen fibers. This provided a ratiometric value of collagen area that was unaffected by sample group variations in cell number and cell size which, as demonstrated here, vary greatly by sex and age. To differentiate the relative contribution of collagen fiber thicknesses, methods were adapted from (Rich & Whittaker, 2005) for use in Fiji (Schindelin et al., 2012). Hue values used for this analysis were as follows: green (52-128), yellow (39-51), orange (10-38), and red (0-9, 230-255). Saturation was not accounted for, and brightness was thresholded at 35-255 to prevent noise from being included in pixel counts. See FIG. 11 for a schematic of this method.

### Intraepidermal Nerve Fiber (IENF) Immunostaining and Quantification

Glabrous hind paw skin was excised and fixed in 2% Zamboni's fixative (Newcomer Supply #1459A) for 2 hrs at room temperature, moved to 30% sucrose in 1×PBS overnight (until tissues sank), and embedded in OCT (Tissue-Tek cat #4583 Miles, Inc.) with orientation noted. Tissues were sectioned at 25 μm onto glass slides. Tissue sections were blocked in 200 μL blocking solution (1×PBS/0.3% Triton X-100/5% BSA) at room temperature for 1 hour and then incubated in primary antibody (Proteintech rabbit anti-PGP9.5 (14730-1-ap), 1:1000) for 1 hour at room temperature and then moved to 4° C. for incubation overnight. Sections were rinsed 3×1 hr in 1×PBS and incubated with goat anti-rabbit IgG Alexa Fluor Plus 594 (1:1000, ThermoFisher Cat #A32740) for 1 hour at room temperature and then moved to 4° C. for incubation overnight. Sections were rinsed with 3×1 hour with 1×PBS and then incubated in 100 ng/mL DAPI solution for 15 minutes. Sections were rinsed 3×10 minutes in distilled H₂O then mounted with a drop of mounting fluid (Prolong Gold, Thermofisher #P36931) and a No. 1.5 glass coverslip for imaging on a Leica Stellaris 5 confocal microscope. Region of interest (ROI) of the epidermis was generated for each image in Fiji (Schindelin et al., 2012). Fluorescence intensity was thresholded in Fiji using the intermodes function to remove skin autofluorescence from measurements. Nerve fiber density was measured as a ratio of PGP9.5 fluorescence to total ROI area for each tissue.

### Neuromuscular Junction (NMJ) Immunostaining and Quantification

Medial gastrocnemius (MG) and soleus (SOL) muscles were excised from male and female HET3 mice. Muscles were fixed in 2% PFA for 2 hrs, washed in 1×PBS, and muscle fibers were teased apart and placed between two glass slides held together by binder clips for 30 min at 4° C. Tissues were then blocked in 1×PBS/2.5% BSA/1% Triton X-100 overnight at 4° C. on rotator. The next day tissues were incubated in primary antibodies 2H3 (1:500, DSHB, Cat #2H3) and SV2 (1:250, DSHB, Cat #SV2) diluted in 1×PBS for 24 hr at 4° C. on rotator. The tissues were washed in 1×PBS for 4 hrs at 4° C. with the 1×PBS replace hourly. Next, tissues were incubated in secondary antibody solution containing α-Bungarotoxin (BTX) Alexa Fluor 555 conjugate (1:1000, ThermoFisher Cat #B35451) and goat anti-mouse IgG1 Alexa Fluor 488 (1:500, ThermoFisher Cat #A-21121) diluted in 1×PBS, incubated 24 hrs at 4° C. on rotator. The next day tissues were washed in 1×PBS for 4 hrs with 1PBS replaced fresh every 1 hr. When necessary, tissues were additionally stained with either SOX10 (1:250, Abcam Cat #ab227680), MPZ (1:250, Abcam, Cat #ab31851) or S100B (1:250, Abcam Cat #ab52642) for 24 hrs at 4° C. on rotator, washed in 1×PBS, incubated in secondary antibody (goat anti-rabbit IgG Alexa Fluor Plus 647, 1:500) for 24 hrs at 4° C. on rotator, and then washed again in 1×PBS. Finally, tissues were placed onto a slide with 3-4 drops of aqueous mountant applied, sealed with coverslip, and imaged on a confocal microscope. Fifty NMJs were counted per tissue and were categorized as being fully occupied or not. The number of tSCs labeled by SOX10 were counted for each of those 50 NMJs when co-staining was performed.

### Microscopy

### Confocal

Confocal micrographs were captured on a Leica Stellaris 5, laser scanning confocal microscope using LASX software. Fluorescent labels were excited with either a diode 405 nm laser: DAPI (ex 405 nm, em 417-570) or a white light laser: Alexa Fluor 488 (ex 499 nm, em 510-570), Alexa Fluor 555 (ex 553 nm, em 565-610 nm), Alexa Fluor Plus 594 (ex 590 nm, em 600-700 nm), Alexa Fluor Plus 647 (ex 653 nm, em 665-720). The scanning speed was set to 400 Hz or 600 Hz. Photons were detected with Power HyD S detectors. Objectives included: HC PL APO 10×/0.40 CS2, HC PL APO 40×/1.30 OIL CS2, and HC PL APO 63×/1.40 OIL CS2. Pinhole Airy 1.00 AU. Confocal zoom was applied to further increase magnification when necessary. Image processing performed in Leica Application Suite X and Fiji software.

### Widefield Epifluorescence

Epifluorescence micrographs were captured on a Nikon Eclipse E400 epifluorescence microscope using a Hamamatsu ORCA-Flash4.0 V2 Digital CMOS monochrome camera. Alexa Fluor 594 fluorophores were excited using a TxRed filter cube and Alexa Fluor 488 was excited with a GFP filter cube. Objectives used: Nikon CFI Plan Apo 10×/0.45 and Nikon CFI Plan Fluor 40×/0.75. Images were captured utilizing the extended depth of field (EDF) function; LUTs were adjusted to improve structural visualization. Post processing was performed in Nikon Elements BR software.

### Brightfield and Polarized

Brightfield and polarized light micrographs were captured on a Nikon Eclipse E400 microscope using a Nikon DS-fi3 color camera. Objectives used: Nikon CFI Plan Apo 4×/0.20, Nikon CFI Plan Apo 10×/0.45, Nikon CFI Plan Apo 20×/0.75 and Nikon CFI Plan Fluor 40×/0.75. LUTs were adjusted to improve structural visualization. Post processing was performed in Nikon Elements BR software.

### Quantitative Real Time PCR

Zymo DirectZol RNA extraction kit (Zymo, Irvine, CA, USA; Cat #R2052) was used for RNA extraction from whole scWAT depot or similarly sized flank skin region. RNA yield was determined using a Nanodrop and cDNA was synthesized using High-Capacity Synthesis Kit (Applied Biosystems, Foster City, CA, USA; Cat #4368813). Real-time quantitative polymerase chain reaction was performed using SYBR Green (Bio-Rad, Cat #1725271) on a CFX384 real-time PCR detection system (Bio-Rad, Hercules, CA, USA). Gene expression was normalized to housekeeper gene Ppia for analysis. Primers used for qPCR are listed in Table 1.

### Whole Adipose Depot Immunostaining, Imaging, and Quantification

Intact scWAT depots were excised from mice, fixed overnight in 2% PFA at 4° C., and processed following the Z-depth reduction method as described previously (Willows et al., 2021) with accompanying protocol (Willows, Blaszkiewicz, & Townsend, 2022). Tissues were stained with the sympathetic nerve marker tyrosine hydroxylase (TH, 1:200, EMD Millipore, AB152), secondary antibody (goat anti-rabbit IgG Alexa Fluor Plus 594, 1:1000, ThermoFisher Cat #A32740) and the vascular marker Isolectin IB4 conjugated to Alexa Fluor 488 (2.5 μg/mL, Thermofisher, Cat #I21411). Whole scWAT tissues were imaged on a Leica Stellaris 5 confocal microscope at 10× or 63× objective magnifications. Whole tissue images were generated by tiling Z-maximum intensity projections of 10× objective magnification micrographs captured at 720×720-pixel resolution with a Z-step size of 10 μm. When tiled together, whole depot image resolutions were roughly 30,000×19,000 pixels and were used for whole depot relative density quantifications and was reduced to an 800 pixel by linear binning (height×width ratio maintained) for visualization in figures. Tissue boundaries were outlined with the polygon sections tool in Fiji to create an ROI of the whole tissue which was used to measure the tissue area for normalization. TH fluorescence intensity was threshold at 35-255 and IB4 fluorescence intensity was threshold at 45-255 to generate masks for relative density measurements.

Fluorescence overlap was measured in five representative images of innervated blood vessels per tissue. Images were captured as 50 μm Z-stacks (2.5 μm step size) at 10× objective magnification at 1024×1024-pixel resolution. A gaussian blur (sigma=80) was subtracted from each channel to remove background and a mask was generated for each channel separately. TH fluorescence intensity threshold was set to 100-255 to generate the mask for quantification. IB4 channel had an unsharp mask applied (radius=1, mask-0.60) and fluorescent intensity threshold was set to 15-255 to generate the first iteration of the mask. To further refine boundaries a binary dilation was applied, a fill holes function was applied, and a median filter (radius=1) was applied. Mander's overlap coefficient was measured between the two generated masks using the Coloc 2 plugin in Fiji and averaged for the five images per tissue.

### Wire Myography

A Danish Myography Technologies A420 (Denmark) was utilized for wire myography experiments as previously described (del Campo & Ferrer, 2015). Mouse protocols for vascular analyses were approved by the Maine Medical Center Institutional Animal Care and Use Committee. Mice were heparinized with 300U heparin injected intraperitoneally (Patterson Veterinary Cat #07-893-7851) 5 min prior to euthanasia. Mouse thoracic aortae with attached PVAT were excised in a 2 mm segment and bathed in physiological saline solution (0.13 M NaCl, 4.7 mM KCl, 1.18 mM KH2PO4, 1.17 mM MgSO47H2O, 5.5 mM glucose, 0.026 mM EDTA, and 1.6 mM CaCl2) on ice. After mounting of with 40 μm wire, vessel segments were bathed in physiological saline solution under oxygenated conditions (95% O2+5% CO2) at 37° C. for 30 minutes. Phenylephrine (Sigma Aldrich P6126) was used for vessel contraction at a dose curve from 2 nM-10 mM. Every consecutive dose was added after vessel change reached a plateau. Vessels were washed twice for 15 minutes to restore basal tone, and pre-contracted to 50-80% of maximum phenylephrine-induced contraction. Acetylcholine (Sigma-Aldrich Cat #A2661) was used in a dose curve from 2 nM-10 mM for relaxation. After acetylcholine-induced vasodilation, vascular integrity was tested using 100 mM KCl, and peak contraction was recorded at 8 minutes post-addition. EC50 calculations were performed using the non-linear regression algorithm from GraphPad Prism v7.

Western blot (WB)

To confirm that the rapamycin treatment was effective at inhibiting mTORC1 in the late-intervention group was analyzed for phosphorylation of p70 S6 kinase following methods described (Lamming et al., 2012). Protein expression was measured by western blot analysis of liver lysates. Livers were homogenized in RIPA buffer with protease inhibitors in a Bullet Blender. A Bradford assay was performed to measure total protein from which equal concentrations of protein lysates were prepared in Laemmli buffer using 1×PBS as diluent. 30 μg of protein were loaded per lane of a 10% polyacrylamide gel, and following gel running, proteins were transferred to PVDF membrane and incubated with 10% Roche Blocking Reagent for 1 hr at room temperature prior to antibody incubation. The membrane was bisected horizontally at 37 kDa to avoid the need for stripping proteins later. Primary antibodies included: p70 S6 Kinase (65kda, 1:1000, Cell Signaling Cat #2708); p-Thr389 p70 S6 Kinase (65kda, 1:1000, Cell Signaling Cat #9234); Cyclophilin B (21 kDa, 1:40,000, Abcam, Cat #16045). Primary antibodies were incubated overnight at 4° C. on a rotator with gentle agitation. Membranes were rinsed with 1×TBS-T and then incubated in anti-rabbit HRP-linked secondary antibody (1:3000, Cell Signaling Technology, Cat #7074) for 1 hr at room temperature. Blots were visualized with enhanced chemiluminescence (ECL; Pierce) on a Syngene G: BOX. Protein expression was normalized to Cyclophilin B and quantified by densitometry in Fiji (Schindelin et al., 2012).

### Statistics

Statistical calculations for determining significance were made in Prism (GraphPad Software). ROUT outlier test (Q=1%) was performed on raw data to identify and remove statistical outliers within data sets. Indicators of statistical significance were unpaired two-tailed Student's t-tests, Mann-Whitney nonparametric test, one- and two-way ANOVAs with Tukey's correction for multiple comparisons, and Kruskal-Wallis nonparametric test with Dunn's correction for multiple comparisons (specified in each figure legend). Nonparametric tests were used for qPCR analysis of HET3 tissues due to a non-gaussian distribution of data points. Linear regression analysis was performed with Goodness of Fit measured by R-squared and the significance of slope determined by F-test. All error bars are SEMs. When p-value is otherwise not directly stated: ^{n.s.}p>0.05, *p<0.05, **p<0.01, ***p<0.001, ****p<0.0001.

### Results

Age-induced changes to adiposity, adipocyte cell size, and adipose tissue inflammation Aging precipitates several phenotypic changes to adipose tissue in both mice and humans, including adipose tissue redistribution (subcutaneous depots shrink while visceral depots expand), chronic inflammation, and reduced WAT browning potential, for example (Mancuso & Bouchard, 2019; Palmer & Kirkland, 2016; Von Bank, Kirsh, & Simcox, 2021). In mice, the majority of these studies were performed using genetically inbred strains such as BL6. The goal was to investigate these age-related changes in genetically diverse HET3 mice that are used for systematic longevity treatment studies and serve as a more appropriate mouse model for human aging. Additional data was also collected in the well-studied BL6 mouse as an inbred-strain reference. Male mice are more prone to weight gain, macrophage infiltration and insulin resistance when placed on a high-fat diet (Chang et al., 2018). Additionally, male rats show an increase in age-related metabolic disturbances, such as insulin resistance and WAT inflammation compared females (Garcia-Carrizo, Priego, Szostaczuk, Palou, & Pico, 2017). Taken together male WAT is less accommodating to energy imbalance and takes on pathophysiological states earlier than in females. For these reasons, the focus was on assessment of aging in BL6 male mice, as a reference for HET3 sex difference assessments.

Male BL6 mice at 15 weeks and 75 weeks (N=7 at each age) were measured for body weight (FIG. 1A), subcutaneous adiposity (scWAT weight/body weight; FIG. 1B), and visceral adiposity (pgWAT weight/body weight; FIG. 1C). Adipose depot weights used for calculating adiposity can be found in FIG. 9. Body weight was increased significantly at 75 weeks of age versus 15 weeks (p=0.0001), which more closely correlated with an increase in subcutaneous adiposity (p=0.0326) and not visceral adiposity (FIG. 1B-C).

Male and female HET3 mice were aged to 13 weeks, 30 weeks, 41 weeks, 62 weeks, 106 weeks, and 126 weeks (N=5-12 per group depending on cohort survival). Body weights and adiposities were measured at these ages and compared by age and sex. Male and female HET3 mice both displayed a significant increase in body weight from 13 weeks to 30 weeks (male, p=0.0022; female, p=0.0128; FIG. 1D). After 30 weeks, males steadily lost body weight, whereas females had a secondary increase in body weight between 41 weeks and 62 weeks (p=0.0008). However, comparing males to females at 62 wks the body weights were the same, but at 13 and 30 weeks of age male mice weighed more than females (FIG. 1E). Therefore, the 41-62-week weight gain in females served to blunt the initial weight difference between the sexes.

Changes in HET3 scWAT adiposity with age mimicked the trends observed in body weight for both sexes (FIG. 1F), with females displaying a much greater proportion of scWAT than males at 62 weeks (p<0.0001) and 106 weeks (p<0.0001; FIG. 1G). By contrast, visceral adiposity continuously decreased with age in both sexes (FIG. 1H), and females had higher visceral adiposity than males at 13 weeks (p=0.0183) and 30 weeks (p-0.0002; FIG. 1I). Taken together, the genetically diverse HET3 mouse line displayed a striking sexual dimorphism in age-related body weight and adiposity trajectories that may cause, or reflect, underlying changes to health outcomes.

In females, irregular hormone cycling signals the start of reproductive senescence which occurs at approximately 8 months (32 weeks of age). In the majority (70%) of mice, this is followed by a polyfollicular anovulatory state of constant estrus characterized by sustained levels of plasma 17ß-estradiol and low levels of progesterone, which is followed by anestrous several weeks later, which is characterized by low circulating ovarian hormones (Diaz Brinton, 2012). It is possible that the increased subcutaneous adiposity observed in females is a result of the increased circulating hormones at those ages, as estrogens have been linked to the promotion, maintenance, and control of adipose tissue metabolism (Pallottini, Bulzomi, Galluzzo, Martini, & Marino, 2008).

Hematoxylin staining of pgWAT and scWAT sections from BL6 male mice demonstrated several age-related changes (FIG. 1J). Cell size appeared visually smaller in both depots at 75 weeks, but was only statistically significant for scWAT (area, p=0.0203; perimeter, p=0.0153; FIG. 1K). Most importantly, pgWAT and scWAT appeared visually distinct from one another at 75 weeks, as scWAT lacked the increase in crown-like structures and lipofuscin that were observed in pgWAT at this age. These provide supporting evidence that aging pathologies can vary between adipose depots (Blaszkiewicz et al., 2020).

HET3 pgWAT tissue sections looked similar to those of BL6, with numerous crown-like structures and patches of lipofuscin appearing at 62 weeks of age and later (FIG. 1L). In general, cell size followed the patterns displayed by changes in adiposity (FIG. 1M), however, several hypertrophic adipocytes were dispersed throughout the tissue and were surrounded by patches of lipofuscin and crown-like structures. This was observed primarily in females (FIG. 1L, female at 106 weeks).

### Aging Impacts Peripheral Nerves in Skin and Muscle

Aging is associated with increased rates of peripheral neuropathy in both mice (Yezierski, 2012) and in humans (Richardson, 2002), including in adipose tissue (Blaszkiewicz et al., 2019). Studies in mice have been limited to investigating neuropathy in inbred-strains and have not yet been undertaken in genetically diverse HET3 mice, despite the likelihood that genetic background may contribute to age-related tissue neuropathy.

Since small fiber peripheral neuropathy in skin and subdermal tissues, the type of neuropathy most associated with aging, is thought to begin with the longest axons that innervate the skin of the extremities, paw skin sensory nerve function was assessed through the standard von Frey tactile sensitivity and nociception test, a classical measure for sensory peripheral neuropathy. This revealed that BL6 males had reduced sensitivity as they aged from 15 weeks to 75 weeks (FIG. 2A). Interestingly, a focused assessment at the branch-point of sexually dimorphic phenotypes in weight and adiposity (62 weeks) showed that female HET3 mice were protected from neuropathy at middle age (62 weeks; FIG. 2B), while at later age (126 weeks) they fared worse than the males. This further supports the observation that females have delayed onset of aging-related morbidities, which are then exacerbated compared to males at late age and are likely linked to hormonal changes after reproductive senescence.

This behavioral nerve testing was supported by histological staining of skin intraepidermal nerve fibers (IENF) using the pan-neuronal marker PGP9.5 (FIG. 2C, area within dashed line) and was quantified as relative nerve fiber area (FIG. 2D). Male HET3 mice had decreased IENF from 35 weeks to 65 weeks of age (p=0.0358; FIG. 2D). Compared to the hind paw skin of 35-week-old mice, in 65-week-old mice the dermal fibers were still evident within the subepidermal neural plexus (FIG. 2C), but a paucity of fine fibers branching into the epidermal layer was noted. However, at a late age (95 weeks old) IENF density increased back to baseline. This pattern of paw skin innervation looks strikingly similar to what was previously observed in BL6 mice (Verdu, Ceballos, Vilches, & Navarro, 2000). It was suspected that the increase in IENF density could be due to a pathologic aberrant outgrowth of small fibers in late-stage peripheral neuropathy (Hirai et al., 2017). This would at least partially explain why an increase in paw innervation in old age was observed, but not an increase in the response to mechanical stimuli.

Finally, neuromuscular junction (NMJ) integrity was assessed by histology and quantification of occupied versus unoccupied junctions in male HET3 mice (FIG. 2E). Staining revealed significant NMJ neuropathy in the medial gastrocnemius (MG) muscle (p=0.0486), but not the soleus (SOL) muscle (FIG. 2F). Similar MG neuropathy was observed in female HET3 mice (p=0.0151) (FIG. 10A). Decreased NMJ occupation by 95 weeks was comparable to what had been shown in BL6 mice previously (Valdez et al., 2010). Co-staining NMJs with the Schwann cell nuclear marker SOX10 was performed to see if the age-related NMJ neuropathy in HET3 mice could be attributed to a loss of terminal Schwann cells (tSCs, or specialized support cells located at the NMJ terminus) as had been proposed previously (Fuertes-Alvarez & Izeta, 2021). At 85 weeks and 115 weeks of age wherein an increase in structurally altered and unoccupied NMJs was noted (FIG. 2G), as anticipated (Taetzsch & Valdez, 2018). However, tSCs were located at all NMJs regardless of NMJ morphology or pre-synaptic occupation (FIG. 2G), and the number of tSCs per NMJ was unchanged among all ages (FIG. 2H). Co-labeling of NMJs was additionally performed with the Schwann cell membrane marker S100ß or the myelin marker MPZ to parse out potential differences with age. Interestingly, the Schwann cells continued to form a path to NMJ's, even when the nerve ending itself was absent in aged animals (FIG. 2I, arrow). This sustained Schwann cell presence appeared to have no impact on de-myelination, as myelin was absent around unoccupied NMJs (FIG. 2J).

### Changes in Adipose Tissue and Skin Gene Expression Varies by Age and Sex.

It was previously reported adipose tissue neuropathy with aging in mouse and human samples (Blaszkiewicz et al., 2019). Now, in order to investigate potential contributions of tissue gene expression changes across aging in the HET3 mouse as factors contributing to age-related adipose neuropathy, a panel of qPCR markers to investigate cytokines (interleukin 4, Il4; interleukin 6, Il6; interleukin 10, Il10; interleukin 13, Il13), angiogenesis markers (platelet endothelial cell adhesion molecule, Pecam1/Cd31; vascular endothelial growth factor a, Vegfa), Schwann cells (Sox10), nerve terminals (post-synaptic density 95, Psd95 (also referred to herein as PSD-95); glutamate ionotropic receptor AMPA type subunit 2, Gria2; Synapsin I, Syn1; Synapsin II, Syn2; Synaptophysin, Syp), mitochondrial respiration (ATP synthase, Atpl; Ubiquinol-cytochrome c reductase, Uqcr; Cytochrome c oxidoreductase Vb, Cox5b; NADH dehydrogenase, Ndufa1), and collagens (collagen type 1 αl, Col1a1; collagen type 2 α1, Col2a1; collagen type 3 α1, Col3a1; collagen type 4 α1, Col4a1; collagen type 5 α1, Col5a1; collagen type 6 α1, Col6a1), was developed.

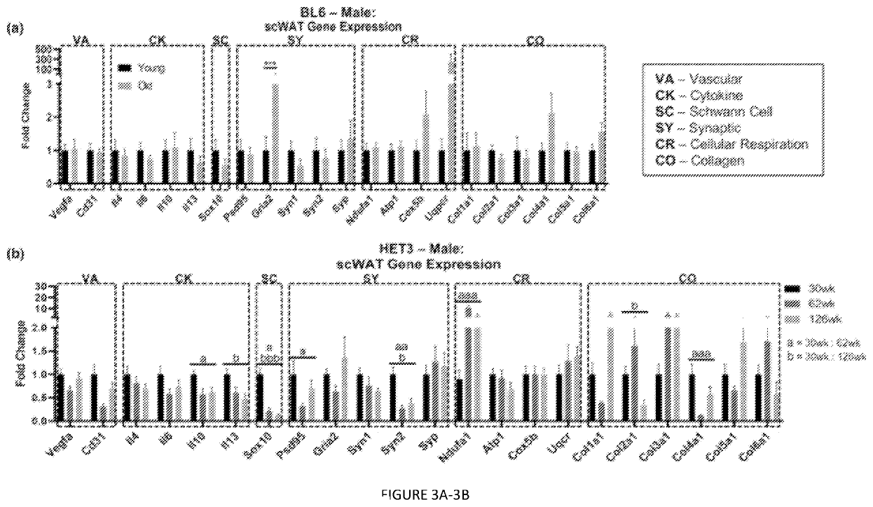

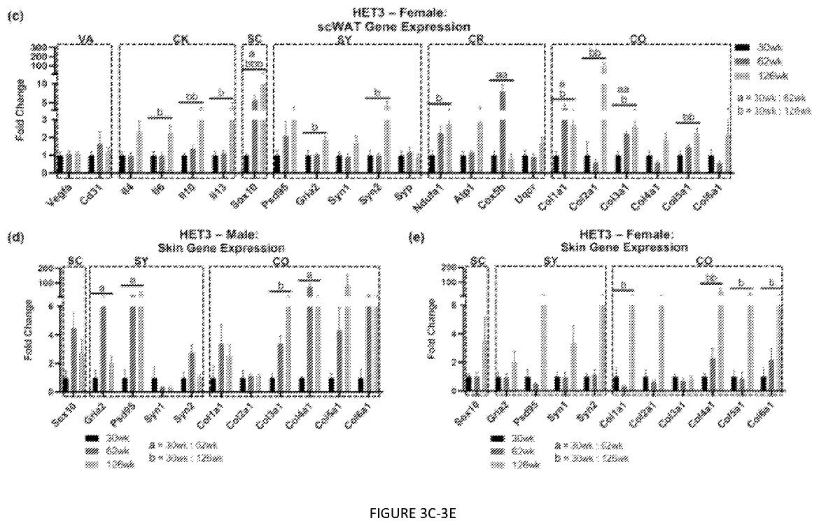

Gene expression in whole scWAT tissue lysates from BL6 (15 weeks; 75 weeks; FIG. 3A) and HET3 (30 weeks; 62 weeks; 126 weeks; FIG. 3B-C) mice, was measured. Interestingly, male BL6 mice only displayed a change in Gria2 expression, which was increased at 75 weeks (p=0.0002). Male HET3 mice displayed a marked decrease in Sox10 and Syn2 (with Syn1 trending down) with advancing age, which may indicate adipose neuropathy. Male HET3 mice displayed a coordinated decreasing trend for all cytokine markers with increasing age, with significant decreases in Il10 and Il13 (FIG. 3B), together suggesting age-induced tissue inflammation. The variation in expression across individuals may be due to the mixed genetic background of this line, and the impacts of genetics on gene expression (Lipman, Galecki, Burke, & Miller, 2004)). Results from female HET3 scWAT gene expression was opposite of the male mice (FIG. 3C), again indicative of protection from age-related pathologies. Cytokine gene expression revealed a coordinated increase for Il4, Il6, Il10 and Il13 at 126 weeks of age, with all but Il4 being statistically significant. In females there was also a marked increase in Sox10 and Syn2 (with Syn1 trending up), indicating compensatory support for tissue innervation. Like BL6 mice, female HET3 mice had increased Gria2 expression, and if this represents loss of tissue nerve endings, the upregulation of Schwann cell and other synaptic markers may indicate enhanced neural plasticity in females to counteract this onset of age-related neuropathy. Mitochondrial markers Ndufa1 and Cox5b increased with age in females, and collagen gene expression increased for all collagen types by 126 weeks, with Col1a1, Col2a1, Col3a1, and Col5a1 all reaching statistical significance (FIG. 3C).

Additionally, in HET3 mice gene expression of many of these same markers was measured in the ‘flank’ skin directly above the inguinal scWAT depot (FIG. 3D-E), since age-related small fiber peripheral neuropathy likely starts at the skin surface (length-dependent neuropathy of the longest axons first) and then progresses down to deeper tissue layers over time. Male mice displayed increased Psd95 expression, which can represent peripheral neuropathy in the tissue since shedding of PSD95 from nerve terminals can occur with loss of tissue innervation and synaptic remodeling (Low & Cheng, 2006). Also observed was an increase in Gria2 expression with age and a trend for increased collagen expression overall, with just Col3a1 and Col4a1 being significant (FIG. 3D). Similar to scWAT, Female HET3 skin displayed consistent increasing trends for synaptic, Schwann cell, and collagen gene expression at 126 weeks of age, but none of these changes were significant (FIG. 3E).

The dissimilarities between male and female HET3 gene expression in scWAT and the overlying skin provided further evidence for sexual dimorphism in aging. Females seem to be protected from mid-age peripheral neuropathy, and increased Schwann cell and synaptic gene expression may be the contributing (or resulting) factors. While direct comparisons cannot be made between BL6 and HET3 mice, the stark contrast in gene expression patterns further emphasized the impact that genetic variability, or lack there-of, may have on age-related pathologies, including neuropathy.

White Adipose Tissue Becomes Fibrotic with Age Regardless of Genetic Background.

Fibrosis is defined as the excess accumulation of extracellular matrix (ECM) components, primarily collagens type I, III, and VI (Sun, Tordjman, Clement, & Scherer, 2013). In fibrotic states in adipose tissues, collagen limits adipocyte cell growth causing constriction and reducing the morphological flexibility that is characteristic of adipose tissue cells. Fibrosis in adipose is especially prevalent with obesity (Khan et al., 2009). This mechanical constriction due to increased ECM can lead to cell damage and chronic inflammation, and adipose tissue fibrosis has been linked to insulin resistance, chronic inflammation, and impaired adipogenesis (Datta, Podolsky, & Atabai, 2018; DeBari & Abbott, 2020; Khan et al., 2009). Much focus has been placed on investigating adipose fibrosis with obesity due high-fat/high-sugar diets (Pincu et al., 2016), but little work has been done with aged adipose, despite the fact that aging has been demonstrated to increase the likelihood of pathological cardiac and pulmonary fibrosis in mice and humans (Murtha et al., 2019). Infrequent studies to date have reported increased fibrosis in visceral and dermal WAT with aging (Donato et al., 2014).

Changes to collagen deposition in both BL6 and HET3 mice in visceral and subcutaneous adipose depots with age were investigated. To do so, a method was developed for quantifying total collagen and the ratio of thin to thick collagen fibers in adipose tissue, which is not confounded by the sex and age specific changes in adipocyte cell size and cell number (FIG. 10). Picrosirius red (PSR) staining was used, which binds to type I, II, and III collagens (Rittie, 2017), coupled with polarized light microscopy to differentiate collagen fiber thickness in the tissue ECM (Lattouf et al., 2014). A striking increase in total collagen was observed in BL6 mouse scWAT with increasing age (FIG. 4A-B, p-0.0014). The color of collagen birefringence indicated the thickness of collagen fibers, with green and yellow representing thin fibers and orange and red representing thick fibers (Rich & Whittaker, 2005). Although collagen fiber thickness has been historically used to distinguish between type I (thick) and type III (thin) collagens (Junqueira, Cossermelli, & Brentani, 1978), there are several instances when this is not a direct correlation (Rich & Whittaker, 2005), so fibers are referred to as ‘thick’ or ‘thin’ in the analyses. With increased age, green birefringence decreased (p=0.0121), while orange birefringence increased (p=0.0108; FIG. 4C), and the ratio of thin to thick collagen (15 weeks, 65%: 35%) decreased significantly with age (75 weeks, 34%: 66%; p=0.0292; FIG. 4D). BL6 pgWAT demonstrated identical changes with age, but less collagen was observed overall (FIG. 4E-H).

HET3 mice appeared to have less scWAT collagen than BL6 mice (FIG. 4I), likely a protective effect of their diverse genetic background, but aging from 35 weeks to 65 weeks increased total collagen in the tissue (p-0.0369; FIG. 4J). The proportion of green birefringence decreased, and orange increased with age (green, p=0.0001; orange, p=0.0007; FIG. 4K). Accordingly, the ratio of thin to thick collagen (35 wk, 85%: 14%) decreased significantly with age (66 weeks, 72%: 28%; p-0.0028; FIG. 4L). In addition to comparing HET3 pgWAT collagen across age, males to females were also compared. Although the females at 62 weeks appeared to become fibrotic (FIG. 4M), this difference was not statistically significant in either sex (FIG. 4N). Trends in collagen birefringence and ratios of thin to thick collagen also lacked statistical significance (FIG. 4O-P). These data confirm that both subcutaneous and visceral adipose depots become fibrotic with age, regardless of the mouse strain, sex, and that scWAT is more affected than pgWAT.

### Aging Impacts on Vasculature

Based on previously reported observations of peripheral nerves around blood vessels in scWAT reducing with age in male BL6 mice (Blaszkiewicz et al., 2019), it was exigent to determine whether this was also observed in a more genetically heterogeneous mouse strain (HET3). First, wire-myography of thoracic aortae from male HET3 mice was performed, at approximately 30 wks, 60 weeks, or >80 weeks of age. Because of the impact of the surrounding perivascular adipose tissue on vascular reactivity, these assays were performed on isolated aortic segments with overlying perivascular adipose intact. No difference between ages were observed in total vasoconstriction when induced with phenylephrine (Phe; FIG. 5A). Vasorelaxation was induced with acetylcholine (Ach) following constriction with Phe, and this did reveal a steady decrease in relaxation potential with age that became significantly blunted at 80 weeks (p=0.0189; FIG. 5B). This was similar to what had been observed in aged rats (Luttrell et al., 2020), and similar to a previous study which demonstrated that B6D2F1 mice (which comprise 50% of the HET3 genome) displayed decreased insulin-stimulated arterial vasodilation in scWAT, pgWAT, and BAT with age (Islam et al., 2020). Perivascular adipose tissue phenotype was assessed across aging, using a previously established reproducible protocol to quantify percentage lipid within mouse PVAT (Tero, Fortier, Soucy, Paquette, & Liaw, 2022). Interestingly, there were no differences in percent lipid in PVAT between mouse ages, but lower lipid in PVAT was associated with body weight (FIG. 11A-B).

To follow up on prior observation of nerves dying-back from ing-scWAT vasculature in aged male BL6 mice the occurrence of neurovascular neuropathy in the HET3 mouse was investigated through quantitative histology. Intact whole inguinal scWAT depots were excised from male HET3 mice (20 week, 60 week, and 100 week-old) and co-stained for tyrosine hydroxylase (TH, the rate-limiting step in catecholamine synthesis) to label sympathetic nerves and Isolectin-Ib4 (IB4) to label blood vessels (a caveat being that IB4 may also label a small subset of non-peptidergic nociceptive neurons, which are easily distinguishable from the vasculature by morphology). The fluorescence area of Z-maximum intensity projections of the whole tissue was measured for nerve and blood vessels. It was found that tissue innervation and vascularity somewhat increased with increased tissue mass (FIG. 12C), contrary to what was observed in the obese, diabetic, and neuropathic BTBR ob/ob mouse model (Wang et al., 2020; Willows, Gunsch, et al., 2022). Potentially, a relatively slow increase in fat mass (as with aging) allows the nerves and vasculature to respond adequately to tissue demands, in contrast with obese states. To account for this slight increase, total innervation and vascularity were normalized to total tissue area, which eliminated any correlation between fluorescence area and tissue mass (FIG. 12C). It was found that age had no impact on total nerve fiber density as averaged across the whole tissue (although regional patterns were observed to be different across age; FIG. 5D), but that tissue vascularity did increase by 100 weeks of age (FIG. 5E). Since the relative innervation of an ing-scWAT depot correlated to the relative vascularity (FIG. 12D), the disconnect between increasing vascularity and unchanged innervation could be reflected in a significantly altered ratio of nerves to blood vessels at 100 weeks. However, this was not the case (FIG. 5F). To investigate further, tissue vasculature was analyzed at higher magnifications to look for signs of neuropathy, such as nerves disassociated from blood vessels, as previously observed in BL6 mice (Blaszkiewicz et al., 2019). No such signs were observed (FIG. 5G) in the HET3 mice, again potentially a protective effect of the mixed genetic background. Quantification of nerve-blood vessel overlap further supported that there was no change in blood vessel innervation with age (FIG. 5H-I). While extensive quantification was only performed in males, histology of female HET3 mice at 62 weeks and 126 weeks also did not reveal any signs of adipose tissue neuropathy (FIG. 12E). Likely, inter-individual differences due to relative genetic contributions of the four founder strains contributed to the blunting of any neuropathic phenotype in adipose compared to BL6 mice, which may be a strain more prone to peripheral neuropathy with aging.

Aging impacts on the Neuro-adipose Nexus (NAN).

With the recent discovery of specialized synaptic vesicle-containing nerve terminals in ing-scWAT, the neuro-adipose nexus (NAN) (Willows et al., 2021; Willows, Gunsch, et al., 2022), it was concluded that the analysis of adipose tissue innervation by assessing changes in NAN structure and/or number with age in 20 week, 60 week, and 100 week-old male HET3 mice. NANs were observed in all ing-scWAT depots regardless of age; visualized by varicose axons clustering around specific adipocytes (visualized by their autofluorescence; FIG. 6A). There was a striking increase in NANs with age, ranging from an average of ~160 per tissue at 20 weeks to ~335 per tissue at 60 weeks old (p=0.0260). By 100 weeks, the number of NANs per tissue returned to baseline at ~144 (p=0.0157; FIG. 6B). The two-fold increase in NANs at middle age was not reflected by nerve fiber density changes (FIG. 5) and moreover, it was found that the number of NANs per tissue did not correlate to ing-scWAT weight (FIG. 13A), relative nerve fiber density (FIG. 13B), nor relative vascular density (FIG. 13C). This provided strong evidence for the coordinated redistribution of tissue innervation in response to changing metabolic needs associated with aging and may underscore that nerve remodeling aberrations and not just loss of total neurite density in the tissue, may indicate a pathologic state in adipose. However, this serves as the first piece of data showcasing the neuroplastic potential of NANs.

Longevity treatment rapamycin triggered significant body-weight loss in males with loss in adiposity only in females.

To investigate the effects of the anti-aging treatment rapamycin on adipose tissue and age-related peripheral neuropathy, male and female HET3 mice were fed rapamycin (42 ppm in the diet) ad libitum for 8 months. Treatment was started at two different ages to gauge effectiveness of an early-intervention (started at ~30 weeks old, N=12), as well as a late-intervention (started at ~72 weeks, N=11-12). At the time of behavioral experiments and tissue collection, mice in the early-intervention group were ~76 weeks old (N=8-12) and those in the late-intervention group were ~120 weeks old (N=3-8). Given the additional aging of the late-intervention group, there was a greater attrition of study animals due to age-related death compared to the early-intervention group, and therefore data could be confounded by survivorship bias.

The starting ages for the early- and late-intervention groups were chosen to resemble the studies conducted by the ITP in which rapamycin treatment was started at either 270 days (38.6 weeks) or 600 days (85.7 weeks) of age, whereby both treatment paradigms provided an overall increase in lifespan (Harrison et al., 2009). This same diet (42 ppm rapa) was previously shown to inhibit mTORC1 in both male and female HET3 mice at old age (>100 wk), as measured by a reduction in phosphorylated ribosomal protein S6 (Ser240/244), a substrate of S6 kinase 1, in visceral adipose tissue. It was also found that circulating rapamycin was equivalent between males and females (Harrison et al., 2009). The effectiveness of this treatment in this study was confirmed by reduced p70S6K phosphorylation in the liver of males in the late-intervention group (FIG. 14A).

Body weights were measured throughout the 8-month treatment (FIGS. 7A-B), and it was found that rapamycin decreased body weight in males more than females. This difference was most prominent in the early-intervention group. Rapamycin treatment decreased subcutaneous adiposity only when started early in life (FIG. 7C-D), with no significant changes to visceral adiposity (FIG. 7E-F) or quadriceps weight (FIG. 7G-H) regardless of when treatment was started.

Hematoxylin staining of scWAT sections revealed that rapamycin increased the presence of crown-like structures, lipofuscin, and likely immune cell infiltration in the early-intervention group (FIG. 71), but this was not observed in the late-intervention group which, although older, unexpectedly had fewer signs of inflammation than the rapamycin treated mice of the early-intervention group (FIG. 7J).

### Diet-Fed Rapamycin Did Not Change the Peripheral Neuropathy Phenotype in Aged HET3 Mice.

A von Frey test was used to gauge the level of peripheral neuropathy in the hind paws of HET3 mice following rapamycin treatment. Early-intervention (FIG. 7K) and late-intervention (FIG. 7L) rapamycin treatments had no impact on sensitivity to mechanical stimuli. A previous study using the hot/cold plate nociceptive test also did not find that rapamycin had any impact on paw sensitivity (Neff et al., 2013). NMJ occupation of the medial gastrocnemius (FIG. 7N) and soleus remained mostly intact for the early-intervention group. This is consistent with occupation observed at similar ages in HET3 mice (FIG. 2E), and this was not further affected by rapamycin (FIG. 7N). Similarly, in the late-intervention group, and consistent with the other observations (FIG. 2E), there was a significant decrease in NMJ occupation due to age which was not mitigated by rapamycin (FIG. 7O). Together, these data indicate that rapamycin neither improves nor worsens age-related peripheral neuropathy.

### Diet-Fed Rapamycin Increased Inflammatory, Schwann Cell, and Synaptic Gene Expression in sc WAT.

Males who started rapamycin treatment early in life displayed significant increases in scWAT gene expression for all measured cytokine (Il4, Il6, Il10, Il13) and Schwann cell genes (Sox10, Mpz) (FIG. 7Q). Synaptic gene expression displayed an overall increase with treatment, with Syp being statistically significant (FIG. 7P). Mitochondrial genes also increased with rapamycin (AtP1, Cox5b, and Atp1 all statistically significant) (FIG. 7P). Col3a1 increased with rapamycin, and no other collagen genes were changed (FIG. 7P). Female mice of the early-intervention group displayed similar gene expression profiles as the males for cytokines, Schwann cells, and synapses; all of which revealed a trend to increase, while collagen genes were relatively unchanged (FIG. 7Q). Interestingly, females displayed a significant increase in the angiogenic Vegfa gene in response to rapamycin, whereas males did not, potentially due to the known link between estrogen action and VEGF expression (Fatima et al., 2017). Mitochondrial gene expression was either unchanged, or significantly decreased (Atp1), in the females with rapamycin (FIG. 7Q). Mice in the late-intervention group displayed similar overall sex specific trends in cytokine, Schwann cell, and synaptic genes, but with fewer gene changes reaching statistical significance (FIG. 7R-S). The increased cytokine gene expression could explain the inflammation observed in the male and female early-intervention group scWAT (FIG. 71) but does not explain why this adipose inflammation appeared diminished or absent in the late-intervention group (FIG. 7J). Additionally, this pro-inflammatory phenotype in scWAT was likely similar to the reduced lifespan of rapamycin treated obese C57BL/KsJleprdb/db mice, which was attributed to an increase in inflammation (Sataranatarajan et al., 2016).

Rapamycin Treatment Started Early in Life Increased scWAT Fibrosis.

Inhibition of mTORC1 with rapamycin has been used as an effective treatment for TGF-α-induced pulmonary fibrosis caused by chronic inflammation (Korfhagen et al., 2009), but is ineffective at treating TGFβ1-induced pulmonary fibrosis. TGFβ1 activity drives mTORC1 phosphorylation of 4E-BP1 and increases ECM distribution, and this phosphorylation by mTORC1 is insensitive to rapamycin (Plate, Guillotin, & Chambers, 2020). It was also found that 14 ppm diet-fed rapamycin treatment caused pancreatic fibrosis in diabetic NONcNZO10/LtJ mice (Reifsnyder, Doty, & Harrison, 2014). Given the interest in whether rapamycin would be able to ameliorate the WAT fibrosis observed with aging, scWAT from male and female HET3 mice were stained for collagen following early and late-interventions with rapamycin treatment.

Mice that had been given rapamycin in the early-intervention group appeared to have greater collagen deposition than the vehicle group (FIG. 8A-B), with 3 out of 8 males and 3 out of 11 females exhibiting severely fibrotic tissue, worse than any observations made with aging alone (FIG. 4). However, the total increase in collagen was not statistically significant between treatment groups (FIG. 8C). Birefringence analysis revealed that in males the rapamycin decreased green fibers (p=0.0001) and increased orange (p=0.0001), similar to what was observed with aging, but this change was more pronounced in males than females (green, p=0.0001; orange, p=0.0001) (FIG. 8D). Males demonstrated a significant increase in thick collagen fibers (orange+red) following rapamycin treatment (Veh, 57%: 43%; Rapa, 25%: 75%; p=0.0007) (FIG. 8E) that was more pronounced than females following rapamycin treatment (p=0.0058) (FIG. 8E). Of note, males treated with rapamycin in the early-intervention group displayed the lowest ratio of thin to thick collagen fibers that were observed thus far. The striking fibrosis observed in a handful of male and female HET3 mice following rapamycin treatment was displayed by comparing representative images of tissues with the lowest recorded total collagen (‘Rapa-Healthy’), to those tissues with the highest total collagen after rapamycin treatment (‘Rapa-Sick’) (FIG. 8F). The exacerbated fibrosis is possibly due to inhibition of mTORC2 as a result of the chronic rapamycin treatment, as mTORC2 regulates cytoskeletal organization (Laplante & Sabatini, 2009). Again, variations within these two subsets of rapamycin-treated mice may be due to underlying genetic contributions from the founder strains.

Mice (116-120 weeks old) that had been given rapamycin in the late-intervention group were less susceptible to the fibrosis-inducing effects of rapamycin treatment. Male mice demonstrated a possible increase in total collagen, whereas the females showed no difference between vehicle and rapamycin treated groups (FIG. 8G-H). Differences observed in the males failed to meet statistical significance (FIG. 8I). Birefringence patterns did not vary by treatment or sex (FIG. 8J) and neither did the ratio of collagen fiber thickness (FIG. 8K). These data support the conclusion that treatment with rapamycin at this dose and duration has the potential of being more harmful to adipose tissue health if started early in life rather than late.

### DISCUSSION

### Age-Induced Changes to Adipose Tissues

With age and across genetic strains numerous differences were observed in pathophysiological changes to adipose tissues. Changes to adipose tissue were also often depot-specific, with an age-induced redistribution of fat mass that prioritized accumulation of visceral adipose tissue (Palmer & Kirkland, 2016). These changes also varied by sex, as observed previously (Bond, Calkin, & Drew, 2021; Hoffman & Valencak, 2021). Though a general increase in fat mass occurred with age (as seen in BL6 mice aged to 75 weeks, FIG. 1B-C), at later ages (~100 weeks) a decrease in fat mass was observed in BL6 mice (Hemmeryckx et al., 2010). These observations were similar in HET3 mice (FIGS. 1G and 1I). Data indicate an age-related decline in adipose tissue total vascularity (Donato et al., 2014) and vascular function (Islam et al., 2020), which fits with the observations that adipose vessel innervation changes with age in BL6 mice, but this was not observed in HET3 mice despite changes to vascular function with age. Aging adipose tissue is also characterized by chronic inflammation (De Carvalho, Justice, Freitas, Kershaw, & Sparks, 2019), and in the studies, this was most prominent in visceral pgWAT for both strains and was only prominent in scWAT following rapamycin treatment. In adipose tissue chronic inflammation is tightly linked with tissue fibrosis. As fat mass increases, the proximity of each adipocyte to the vasculature decreases, driving tissue hypoxia and an increase in hypoxia inducible factor (HIF)-1a which induces tissue fibrosis and inflammation (Halberg et al., 2009). This has primarily been observed and described in obese tissues (Crewe, An, & Scherer, 2017), but similar mechanisms likely occur in aging adipose as well (Zhang et al., 2011). The fibrotic and now inflexible EMC applies shear stress to aging adipocytes as they attempt to increase in size. This causes cell damage, lysis, and an increased inflammatory profile (Khan et al., 2009). Subsequently, increased tissue inflammation can also induce tissue fibrosis since many cytokines, including IL-13, can activate macrophages or fibroblasts (Wynn, 2008) which can secrete collagen (Kendall & Feghali-Bostwick, 2014). Both BL6 and HET3 mice displayed this inflamed and fibrotic aged adipose phenotype, as demonstrated by histological assessment (FIG. 1 and FIG. 4), and HET3 mice displayed increased cytokine and collagen gene expression with age (FIG. 3). The fibrotic phenotype was most prevalent in scWAT, potentially due to the larger amount of basal collagen that was observed at a younger age. Previous research in mice on a high fat diet hinted at the possibility that females could be protected from adipose fibrosis onset due their greater ability to expand adiposity (Wynn, 2008), which likely also occurs with aging. However, this was not observed in the aging HET3 mice, suggesting that this protection is limited to instances of increased energy intake alone or may be impacted by genetic strain.

In addition to displaying increased total collagen deposition, the relative proportions of thin and thick collagen fibers also changed with age. Young mice, regardless of sex and strain, displayed primarily thin collagen fibers, while aged mice showed an increase in thick collagen fibers (FIG. 4). While types I, III, and V collagens all constitute the thick fibrils (Spiess & Zorn, 2007), and type I collagen is generally considered to be thicker than type III (Rich & Whittaker, 2005), birefringence of collagen thickness does not necessarily differentiate between these collagen types. The proportion of type I to type III collagen fibers increases in many tissues with age (Mays, Bishop, & Laurent, 1988) and in instances of fibrosis (Ganganna, Shetty, & Shroff, 2012; McKleroy, Lee, & Atabai, 2013). In some cases, these findings were based on incorrectly classifying thick-fiber birefringence as exclusively type I collagen (Tzortzaki et al., 2006). Regardless, these published data coupled with the new findings (FIG. 4) support that aging adipose is distinguished by fibrosis that is characterized by the accumulation of thick collagen fibers, which are possibly more restrictive than thin fibers, further driving shear stress on adipocytes and exacerbating secondary inflammation.

### Age-Related Peripheral Neuropathy

It has been demonstrated that both male and female HET3 mice become neuropathic with age, findings that are consistent with data from BL6 mice presented here and elsewhere (Valdez et al., 2010; Verdu et al., 2000). The data supports a model in which age-related peripheral neuropathy has initial onset in the skin at 65 weeks in males (later in females) and gradually proceeds to the muscle at later ages. This is supported by the observations of reduced sensitivity and nerve fiber density in hind paws at 65 weeks (FIG. 2), and a later reduction in NMJ occupation at 95 weeks (~98% occupation). However, the onset of adipose neuropathy is unclear. It was previously found that by 65 weeks of age BL6 mice displayed a reduction in total innervation of their scWAT depot that was prominent around tissue vasculature (Blaszkiewicz et al., 2019). A consistent loss of adipose vessel innervation was not observed in HET3 mice (FIGS. 5 and 12E) possibly due to genetic background. Regardless, the neuropathic phenotype in adipose is far more prevalent in BL6 mice and appears to be blunted by the genetic diversity of the HET3 mice. Why this blunting was observed for neuropathy in scWAT and not in skin or muscle remains unclear and may be due to other complex changes occurring in adipose with aging, or the fact that total innervation density is more easily quantified in skin and muscle.

PSD95 is a biomarker for aging-related neuropathy. In male HET3 mice, first signs of neuropathy were observed in the skin at about 65 weeks (IENF and von Frey data, FIG. 2). This was accompanied by increased expression of Psd95 in the flank skin (FIG. 3) in the males, indicating that higher Psd95 reflects a more neuropathic tissue. Females, which are found to be at least partially protected from neuropathy (FIG. 2) at this same age, showed no difference in flank skin Psd95 at this age, but a trend for increased levels at 126 weeks when behavioral signs of neuropathy were observed (FIG. 4). Adipose neuropathy in male or female HET3 mice was not observed by histology, and similarly a significant increase was not observed in scWAT Psd95 expression in either sex across ages. Rapamycin treatment, which was unable to attenuate any neuropathic phenotypes, also did not alter levels of scWAT Psd95 expression (FIG. 6). Psd95 appears to be upregulated in neuropathic tissues to compensate for the loss of pre-synaptic nerves and terminals, and this compensation eventually wanes (FIG. 4D), or it is shed as a biomarker of peripheral neuropathy, as is seen in other neurodegenerative states like Alzheimer's Disease (Kivisäkk et al., 2021). Alternatively, PSD95 can be upregulated as an attempt at nerve recovery and regrowth, as is seen after sciatic nerve crush (Gao et al., 2008). Changes in pre-synaptic gene expression (Syn1, Syn2, and Syp) are also observed, and appear to be sex, age, tissue, and strain specific (FIG. 3). Schwann cell gene expression in scWAT decreased in males across aging, but increased in female adipose across aging, and was restored in males by rapamycin treatment, indicating that Schwann cells can be another biomarker for adipose neuropathy.

### Genetic and Sex-Dependent Differences

While direct comparisons cannot be made between BL6 and HET3 mice due to differences in ages across experimental cohorts, many adipose tissue similarities were noted between the strains as they aged, which were summarized with available literature from these aged mice in Table 2. At approximately middle age (65-75 weeks old), BL6 and HET3 mice had similar adipose tissue distribution, cell size, inflammation, and fibrosis. At similar ages, both BL6 and HET3 mice also displayed neuropathy in the skin and muscle. The only substantial differences between genetic strains were specific to scWAT, namely gene expression and neurovascular innervation, and these appeared to demonstrate protection in the HET3 model, likely due to the greater genetic diversity in this strain.

By contrast, sexual dimorphism was prominent in the HET3 mice; also summarized in Table 2. Males and females displayed different patterns of adiposity changes across aging, and females had a delayed onset of skin neuropathy (as has previously been observed in female BTBR ob/ob mice as well (O'Brien et al., 2015). Additionally, while males had decreased synaptic and Schwann cell gene expression with aging, the females had an increase, potentially conferring a protective benefit. The key age for neuropathy onset in males was 62 weeks, and this was significantly later in females-likely due to reproductive senescence, although the NMJ neuropathy onset occurred at similar ages between the sexes. The striking scWAT and flank skin gene expression differences between males and females possibly related to the metabolic protection female mice display in their adipose depots (Chang et al., 2018) at middle age, which could be mediated in large part by estrogen (Bjune, Stromland, Jersin, Mellgren, & Dankel, 2022). Additionally, the hormone fluctuations in female mice at middle to late ages (Diaz Brinton, 2012) likely contributes to the patterns of weight gain and may be pivotal in mediating protection from neuropathic phenotypes until late age when female mice lose estrogen.

### Effects of Rapamycin

Although rapamycin has been demonstrated time and again to be a reliable means of increasing mean lifespan, there is mixed evidence supporting rapamycin as a means of combating aging-related health phenotypes. While some data suggest that rapamycin can slow aging deficits in spontaneous activity and various age-related organ and tissue alterations (Wilkinson et al., 2012), there is also mounting evidence that rapamycin does not extend lifespan by combatting all the pathologies of the aging phenotype, but instead by suppressing cancers (Ehninger, Neff, & Xie, 2014; Neff et al., 2013). The data presented here supports the latter conjecture, as rapamycin had no mitigating effects on age-related declines in tactile sensitivity, adipose tissue pathology, or NMJ occupation as summarized in Table 2.

While neither early-nor late-intervention with chronic rapamycin provided any benefit to nerve integrity or responsiveness, aside from increasing expression of some Schwann cell and synaptic genes, the early-intervention treatment proved to have a more detrimental impact on scWAT than the late-intervention, potentially due to the chronic treatment causing off-target effects on mTORC2 that led to diabetic complications, as reported previously. Cytokine gene expression increased regardless of sex or age of treatment onset, but histological assessment revealed that the early-intervention rapamycin group displayed a striking pro-inflammatory phenotype, coupled with greater thick-fiber collagen deposition. A subset of both male and female HET3 mice treated with rapamycin in the early-intervention group displayed aberrant fibrosis and tissue inflammation when compared to the other mice in their group. Potentially the precise genetic contribution of the four inbred founder strains could explain the variation of this phenotype across HET3 individuals. Genetic loci mapping (Lipman et al., 2004) of the HET3 mice with exacerbated collagen deposition could provide important insights to the genetic factors that predispose mice, or even humans, to the “rapa-sick” scWAT phenotype observed in some mice, but this is beyond the scope of the current study.

When interpreting these results, it is emphasized that the fact that this study chose to use the highest effective dose of diet-fed encapsulated rapamycin as previously tested by the ITP, for reasons described above. Chronic treatment of 42 ppm rapamycin, in addition to having the greatest increase in mean lifespan, also has some of the most dramatic negative side effects through chronic off-target inhibition of mTORC2. Known side effects such as glucose intolerance can be mitigated by decreasing the concentrations of rapamycin (Miller et al., 2014) or by intermittent dosing (Arriola Apelo et al., 2016). Here is provided evidence that a chronic diet-fed rapamycin intervention started late in life (~72 weeks) caused less adipose tissue inflammation and fibrosis than an intervention started early in life (~30 weeks), while both interventions increase lifespan (Harrison et al., 2009), indicating that this can be a preferable treatment approach in order to promote healthy lifespan.

### CONCLUSIONS

While striking impacts were observed on adipose tissue and peripheral neuropathy pathophysiology with aging, these differed by sex and genetic strain, and were not mitigated by rapamycin longevity treatment. Taken together, it has been demonstrated that despite rapamycin's success as a longevity agent in male and female animals, it may be uncoupled from health outcomes, and depending on dose and duration it may reduce the health of adipose tissue and not mitigate peripheral nervous system degradation with aging.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

TABLE 1
List of qPCR Primers.

| | | SEQ | | SEQ |
|---|---|---|---|---|
| GENE | FORWARD SEQUENCE 5′ to 3′ | ID NO | REVERSE SEQUENCE 5′ to 3′ | ID NO |
| Vegfa | AACAAAGCCAGAAAATCACTGT | 1 | CGGATCTTGGACAAACAAATGC | 2 |
| Cd31 | ACGCTGGTGCTCTATGCAAG | 3 | TCAGTTGCTGCCCATTGCATCA | 4 |
| I14 | GGTCAACCCCCAGCTAGT | 5 | GCCGATGATCTCTCTCAAGTGAT | 6 |
| I16 | TAGTCCTTCCTACCCCAATTTC | 7 | TTGGTCCTTAGCCACTCCTTC | 8 |
| I110 | CTATGCTGCCTGCTCTTACTGAC | 9 | CGGAGAGAGGTACAAACGAGG | 10 |
| I113 | CCTGGCTCTTGCTTGCCTT | 11 | GGTCTTGTGTGATGTTGCTCA | 12 |
| Sox10 | AGATCCAGTTCCGTGTCAATAA | 13 | GCGAGAAGAAGGCTAGGTG | 14 |
| Mpz | GTCAAGTCCCCCAGTAGAA | 15 | AGGAGCAAGAGGAAAGCAC | 16 |
| Psd95 | GGACATTCAGGCCAAG | 17 | TCCCGTAGAGGTGGCTGTTG | 18 |
| Gria2 | GCGGTGCTAAAATCGAATGC | 19 | ACAGAGAGGGGCAGGCAGT | 20 |
| Syn1 | CCACAGGGTATGTTGTGCTG | 21 | GCCCAGATGGTTCGACTACA | 22 |
| Syn2 | GCCACCAGGTTAAGCTCTGA | 23 | TTCCAGGAAGGCCAAGGT | 24 |
| Syp | TGACTTCAGGACTCAACACCTC | 25 | CAGGAGCTGGTTGCTTTTCT | 26 |
| Alp1 | CGGTTCAGGTCTCTCCACTC | 27 | TGACGCCTCACTTGAGAATG | 28 |
| Uqpcr | GGGGTGACCCTGAGTATTGA | 29 | ATGTAAGGCACCCAGTCCAG | 30 |
| Cox5b | CAGAAGGGACTGGACCCATA | 31 | ATAACACAGGGGCTCAGTGG | 32 |
| Ndufa1 | GCGGAGCCAGATGTTAAAAA | 33 | CCATCCACCATCTGACACTG | 34 |
| Col1 | TTCTCCTGGCAAAGACGGACTCAA | 35 | GGAAGCTGAAGTCATAACCGCCA | 36 |
| Col2 | AAGGGACACCGAGGTTTCACTGG | 37 | GGGCCTGTTTCTCCTGAGCGT | 38 |
| Col3 | GCCCACAGCCTTCTACACCT | 39 | GCCAGGGTCACCATTTCTC | 40 |
| Col4 | CTCACTGTGGATCGGCTATTC | 41 | CGCTTCTAAACTCTTCCAGACA | 42 |
| Col5 | CTTCGCCGCTACTCCTGTTC | 43 | CCCTGAGGGCAAATTGTGAAAA | 44 |
| Col6 | ACAGTGACGAGGTGGAGATCA | 45 | GATAGCGCAGTCGGTGTAGG | 46 |
| Ppia | CAAATGCTGGACCAAACACAA | 47 | AAGACCACATGCTTGCCAT | 48 |

TABLE 2
PRIMERS TO MEASURE PSD-95

| PRIMER | SEQUENCE in 5′ to 3′ | TM | SEQ ID NO |
|---|---|---|---|
| Mouse PSD-95 | GGACATTCAGGCCAAG | 56.9 | 17 |
| Forward | | | |
| Mouse PSD-95 | TCCCGTAGAGGTGGCTGTTG | 59.1 | 18 |
| Reverse | | | |
| Human PSD-95 | TGAGATCAGTCATAGCAGCTACT | 54.9 | 49 |
| Forward | | | |
| Human PSD-95 | CTTCCTCCCCTAGCAGGTCC | 59.1 | 50 |
| Reverse | | | |

### REFERENCES

1. Arriola Apelo, S. I., Neuman, J. C., Baar, E. L., Syed, F. A., Cummings, N. E., Brar, H. K., . . . Lamming, D. W. (2016). Alternative rapamycin treatment regimens mitigate the impact of rapamycin on glucose homeostasis and the immune system. Aging Cell, 15 (1), 28-38. doi: 10.1111/acel. 12405
2. Bjune, J. I., Stromland, P. P., Jersin, R. A., Mellgren, G., & Dankel, S. N. (2022). Metabolic and Epigenetic Regulation by Estrogen in Adipocytes. Front Endocrinol (Lausanne), 13, 828780. doi: 10.3389/fendo.2022.828780
3. Blaszkiewicz, M., Willows, J. W., Dubois, A. L., Waible, S., DiBello, K., Lyons, L. L., . . . Townsend, K. L. (2019). Neuropathy and neural plasticity in the subcutaneous white adipose depot. PLOS One, 14 (9), e0221766. doi: 10.1371/journal.pone.0221766
4. Blaszkiewicz, M., Wood, E., Koizar, S., Willows, J., Anderson, R., Tseng, Y. H., Townsend, K. L. (2020). The involvement of neuroimmune cells in adipose innervation. Mol Med,
26 (1), 126. doi: 10.1186/s10020-020-00254-3
5. Bond, S. T., Calkin, A. C., & Drew, B. G. (2021). Sex differences in white adipose tissue expansion: emerging molecular mechanisms. Clin Sci (Lond), 135 (24), 2691-2708. doi: 10.1042/CS20210086
6. Brisset, M., & Nicolas, G. (2018). Peripheral neuropathies and aging. Geriatr Psychol Neuropsychiatr Vieil, 16 (4), 409-413. doi: 10.1684/pnv.2018.0768
7. Chang, E., Varghese, M., & Singer, K. (2018). Gender and Sex Differences in Adipose Tissue. Curr Diab Rep, 18 (9), 69. doi: 10.1007/s11892-018-1031-3
8. Crewe, C., An, Y. A., & Scherer, P. E. (2017). The ominous triad of adipose tissue dysfunction: inflammation, fibrosis, and impaired angiogenesis. J Clin Invest, 127 (1), 74-82. doi: 10.1172/JCI88883
9. Datta, R., Podolsky, M. J., & Atabai, K. (2018). Fat fibrosis: friend or foe? JCI Insight, 3 (19). doi: 10.1172/jci.insight. 122289
10. De Carvalho, F. G., Justice, J. N., Freitas, E. C., Kershaw, E. E., & Sparks, L. M. (2019). Adipose Tissue Quality in Aging: How Structural and Functional Aspects of Adipose Tissue Impact Skeletal Muscle Quality. Nutrients, 11 (11). doi: 10.3390/nu11112553
11. DeBari, M. K., & Abbott, R. D. (2020). Adipose Tissue Fibrosis: Mechanisms, Models, and Importance. Int J Mol Sci, 21 (17). doi: 10.3390/ijms21176030
12. DeFronzo, R. A. (1981). Glucose intolerance and aging. Diabetes Care, 4 (4), 493-501. Retrieved from PM: 7049632
13. del Campo, L., & Ferrer, M. (2015). Wire Myography to Study Vascular Tone and Vascular Structure of Isolated Mouse Arteries. Methods Mol Biol, 1339, 255-276. doi: 10.1007/978-1-4939-2929-0_18
14 Diaz Brinton, R. (2012). Minireview: translational animal models of human menopause: challenges and emerging opportunities. Endocrinology, 153 (8), 3571-3578. doi: 10.1210/en.2012-1340
15. Donato, A. J., Henson, G. D., Hart, C. R., Layec, G., Trinity, J. D., Bramwell, R. C., . . . Lesniewski, L. A. (2014). The impact of ageing on adipose structure, function and vasculature in the B6D2F1 mouse: evidence of significant multisystem dysfunction. J Physiol, 592 (18), 4083-4096. doi: 10.1113/jphysiol.2014.274175
16. Ehninger, D., Neff, F., & Xie, K. (2014). Longevity, aging and rapamycin. Cell Mol Life Sci, 71 (22), 4325-4346. doi: 10.1007/s00018-014-1677-1
17. Fatima, L. A., Campello, R. S., Santos, R. S., Freitas, H. S., Frank, A. P., Machado, U. F., & Clegg, D. J. (2017). Estrogen receptor 1 (ESR1) regulates VEGFA in adipose tissue. Sci Rep, 7 (1), 16716. doi: 10.1038/s41598-017-16686-7
18. Flurkey, K., Astle, C. M., & Harrison, D. E. (2010). Life extension by diet restriction and N-acetyl-L-cysteine in genetically heterogeneous mice. J Gerontol A Biol Sci Med Sci, 65 (12), 1275-1284. doi: 10.1093/gerona/glq155
19. Fuertes-Alvarez, S., & Izeta, A. (2021). Terminal Schwann Cell Aging: Implications for Age-Associated Neuromuscular Dysfunction. Aging Dis, 12 (2), 494-514. doi: 10.14336/AD.2020.0708
20. Ganganna, K., Shetty, P., & Shroff, S. E. (2012). Collagen in histologic stages of oral submucous fibrosis: A polarizing microscopic study. J Oral Maxillofac Pathol, 16 (2), 162-166. doi: 10.4103/0973-029X.98446
21. Gao, S., Fei, M., Cheng, C., Yu, X., Chen, M., Shi, S., . . . Shen, A. (2008). Spatiotemporal expression of PSD-95 and nNOS after rat sciatic nerve injury. Neurochem Res, 33 (6), 1090-1100. doi: 10.1007/s11064-007-9555-y
22. Garcia-Carrizo, F., Priego, T., Szostaczuk, N., Palou, A., & Pico, C. (2017). Sexual Dimorphism in the Age-Induced Insulin Resistance, Liver Steatosis, and Adipose Tissue Function in Rats. Front Physiol, 8, 445. doi: 10.3389/fphys.2017.00445
23. Halberg, N., Khan, T., Trujillo, M. E., Wernstedt-Asterholm, I., Attie, A. D., Sherwani, S., . . . Scherer, P. E. (2009). Hypoxia-inducible factor 1alpha induces fibrosis and insulin resistance in white adipose tissue. Mol Cell Biol, 29 (16), 4467-4483. doi: 10.1128/MCB.00192-09
24. Harrison, D. E., Strong, R., Sharp, Z. D., Nelson, J. F., Astle, C. M., Flurkey, K., . . . Miller, R. A. (2009). Rapamycin fed late in life extends lifespan in genetically heterogeneous mice. Nature, 460 (7253), 392-395. doi: 10.1038/nature08221
25. Hemmeryckx, B., Loeckx, D., Dresselaers, T., Himmelreich, U., Hoylaerts, M. F., & Lijnen, H. R. (2010). Age-associated adaptations in murine adipose tissues. Endocr J, 57 (10), 925-930. doi: 10.1507/endocrj.k10e-179
26. Hirai, T., Mulpuri, Y., Cheng, Y., Xia, Z., Li, W., Ruangsri, S., . . . Nishimura, I. (2017). Aberrant plasticity of peripheral sensory axons in a painful neuropathy. Sci Rep, 7 (1), 3407. doi: 10.1038/s41598-017-03390-9
27. Hoffman, J. M., & Valencak, T. G. (2021). Sex differences and aging: Is there a role of brown adipose tissue? Mol Cell Endocrinol, 531, 111310. doi: 10.1016/j.mce.2021.111310
28. Islam, M. T., Henson, G. D., Machin, D. R., Bramwell, R. C., Donato, A. J., & Lesniewski, L. A. (2020). Aging differentially impacts vasodilation and angiogenesis in arteries from the white and brown adipose tissues. Exp Gerontol, 142, 111126. doi: 10.1016/j.exger.2020.111126
29. Junqueira, L. C., Cossermelli, W., & Brentani, R. (1978). Differential staining of collagens type I, II and III by Sirius Red and polarization microscopy. Arch Histol Jpn, 41 (3), 267-274. doi: 10.1679/aohc1950.41.267
30. Karastergiou, K., Smith, S. R., Greenberg, A. S., & Fried, S. K. (2012). Sex differences in human adipose tissues—the biology of pear shape. Biol Sex Differ, 3 (1), 13. doi: 10.1186/2042-6410 March 13
31. Kendall, R. T., & Feghali-Bostwick, C. A. (2014). Fibroblasts in fibrosis: novel roles and mediators. Front Pharmacol, 5, 123. doi: 10.3389/fphar.2014.00123
32. Khan, T., Muise, E. S., Iyengar, P., Wang, Z. V., Chandalia, M., Abate, N., . . . Scherer, P. E. (2009). Metabolic dysregulation and adipose tissue fibrosis: role of collagen VI. Mol Cell Biol, 29 (6), 1575-1591. doi: 10.1128/MCB.01300-08
33. Kirkman, M. S., Briscoe, V. J., Clark, N., Florez, H., Haas, L. B., Halter, J. B., . . . Swift, C. S. (2012). Diabetes in older adults. Diabetes Care, 35 (12), 2650-2664. doi: 10.2337/dc12-1801
34. Kivisakk, P., Carlyle, B. C., Ramirez, C. E., Trombetta, B. A., Mendes, M., Brock, M., . . . Arnold, S. E. (2021). Levels of the synaptic proteins PSD-95, SNAP-25, and neurogranin are selectively increased in the cerebrospinal fluid of patients with Alzheimer's disease. Alzheimer's & Dementia, 17 (S5), e056339. doi: https://doi.org/10.1002/alz.056339
35. Korfhagen, T. R., Le Cras, T. D., Davidson, C. R., Schmidt, S. M., Ikegami, M., Whitsett, J. A., & Hardie, W. D. (2009). Rapamycin prevents transforming growth factor-alpha-induced pulmonary fibrosis. Am J Respir Cell Mol Biol, 41 (5), 562-572. doi: 10.1165/rcmb.2008-03770C
36. Lamming, D. W., Ye, L., Astle, C. M., Baur, J. A., Sabatini, D. M., & Harrison, D. E. (2013). Young and old genetically heterogeneous HET3 mice on a rapamycin diet are glucose intolerant but insulin sensitive. Aging Cell, 12 (4), 712-718. doi: 10.1111/acel. 12097
37. Lamming, D. W., Ye, L., Katajisto, P., Goncalves, M. D., Saitoh, M., Stevens, D. M., . . . Baur, J. A. (2012). Rapamycin-induced insulin resistance is mediated by mTORC2 loss and uncoupled from longevity. Science, 335 (6076), 1638-1643. doi: 10.1126/science.1215135
38. Laplante, M., & Sabatini, D. M. (2009). mTOR signaling at a glance. J Cell Sci, 122 (Pt 20), 3589-3594. doi: 10.1242/jcs.051011
39. Lattouf, R., Younes, R., Lutomski, D., Naaman, N., Godeau, G., Senni, K., & Changotade, S. (2014). Picrosirius red staining: a useful tool to appraise collagen networks in normal and pathological tissues. J Histochem Cytochem, 62 (10), 751-758. doi: 10.1369/0022155414545787
40. Lipman, R., Galecki, A., Burke, D. T., & Miller, R. A. (2004). Genetic loci that influence cause of death in a heterogeneous mouse stock. J Gerontol A Biol Sci Med Sci, 59 (10), 977-983. doi: 10.1093/gerona/59.10.b977
41. Low, L. K., & Cheng, H. J. (2006). Axon pruning: an essential step underlying the developmental plasticity of neuronal connections. Philos Trans R Soc Lond B Biol Sci, 361 (1473), 1531-1544. doi: 10.1098/rstb.2006.1883
42. Lumish, H. S., O'Reilly, M., & Reilly, M. P. (2020). Sex Differences in Genomic Drivers of Adipose Distribution and Related Cardiometabolic Disorders: Opportunities for Precision Medicine. Arterioscler Thromb Vasc Biol, 40 (1), 45-60. doi: 10.1161/ATVBAHA.119.313154
43. Luttrell, M., Kim, H., Shin, S. Y., Holly, D., Massett, M. P., & Woodman, C. R. (2020). Heterogeneous effect of aging on vasorelaxation responses in large and small arteries. Physiol Rep, 8 (1), e14341. doi: 10.14814/phy2.14341
44. Mancuso, P., & Bouchard, B. (2019). The Impact of Aging on Adipose Function and Adipokine Synthesis. Front Endocrinol (Lausanne), 10, 137. doi: 10.3389/fendo.2019.00137
45. Mays, P. K., Bishop, J. E., & Laurent, G. J. (1988). Age-related changes in the proportion of types I and III collagen. Mech Ageing Dev, 45 (3), 203-212. doi: 10.1016/0047-6374 (88) 90002-4
46. McKleroy, W., Lee, T. H., & Atabai, K. (2013). Always cleave up y
47. mess: targeting collagen degradation to treat tissue fibrosis. Am J Physiol Lung Cell Mol Physiol, 304 (11), L709-721. doi: 10.1152/ajplung.00418.2012
48. Miller, R. A., Burke, D., & Nadon, N. (1999). Announcement: four-way cross mouse stocks: a new, genetically heterogeneous resource for aging research. J Gerontol A Biol Sci Med Sci, 54 (8), B358-360. doi: 10.1093/gerona/54.8.b358
49. Miller, R. A., Harrison, D. E., Astle, C. M., Baur, J. A., Boyd, A. R., de Cabo, R., . . . Strong, R. (2011). Rapamycin, but not resveratrol or simvastatin, extends life span of genetically heterogeneous mice. J Gerontol A Biol Sci Med Sci, 66 (2), 191-201. doi: 10.1093/gerona/glq178
50. Miller, R. A., Harrison, D. E., Astle, C. M., Fernandez, E., Flurkey, K., Han, M., . . . Strong, R. (2014). Rapamycin-mediated lifespan increase in mice is dose and sex dependent and metabolically distinct from dietary restriction. Aging Cell, 13 (3), 468-477. doi: 10.1111/acel. 12194
51. Miller, R. A., Harrison, D. E., Astle, C. M., Floyd, R. A., Flurkey, K., Hensley, K. L., . . . Strong, R. (2007). An Aging Interventions Testing Program: study design and interim report. Aging Cell, 6 (4), 565-575. doi: 10.1111/j.1474-9726.2007.00311.x
52. Murtha, L. A., Morten, M., Schuliga, M. J., Mabotuwana, N. S., Hardy, S. A., Waters, D. W., Boyle, A. J. (2019). The Role of Pathological Aging in Cardiac and Pulmonary Fibrosis. Aging Dis, 10 (2), 419-428. doi: 10.14336/AD.2018.0601
53. Neff, F., Flores-Dominguez, D., Ryan, D. P., Horsch, M., Schroder, S., Adler, T., Ehninger, D. (2013). Rapamycin extends murine lifespan but has limited effects on aging. J Clin Invest, 123 (8), 3272-3291. doi: 10.1172/JCI67674
54. Niccoli, T., & Partridge, L. (2012). Ageing as a risk factor for disease. Curr Biol, 22 (17), R741-752. doi: 10.1016/j.cub.2012.07.024
55. North, B. J., & Sinclair, D. A. (2012). The intersection between aging and cardiovascular disease. Circ Res, 110 (8), 1097-1108. doi: 10.1161/CIRCRESAHA.111.246876
56. O'Brien, P. D., Hur, J., Hayes, J. M., Backus, C., Sakowski, S. A., & Feldman, E. L. (2015). BTBR ob/ob mice as a novel diabetic neuropathy model: Neurological characterization and gene expression analyses. Neurobiol Dis, 73, 348-355. doi: 10.1016/j.nbd.2014.10.015
57. Pallottini, V., Bulzomi, P., Galluzzo, P., Martini, C., & Marino, M. (2008). Estrogen regulation of adipose tissue functions: involvement of estrogen receptor isoforms. Infect Disord Drug Targets, 8 (1), 52-60. doi: 10.2174/187152608784139631
58. Palmer, A. K., & Kirkland, J. L. (2016). Aging and adipose tissue: potential interventions for diabetes and regenerative medicine. Exp Gerontol, 86, 97-105. doi: 10.1016/j.exger.2016.02.013
59. Pincu, Y., Huntsman, H. D., Zou, K., De Lisio, M., Mahmassani, Z. S., Munroe, M. R., Boppart, M. D. (2016). Diet-induced obesity regulates adipose-resident stromal cell quantity and extracellular matrix gene expression. Stem Cell Res, 17 (1), 181-190. doi: 10.1016/j.scr.2016.07.002
60. Plate, M., Guillotin, D., & Chambers, R. C. (2020). The promise of mTOR as a therapeutic target pathway in idiopathic pulmonary fibrosis. Eur Respir Rev, 29 (157). doi: 10.1183/16000617.0269-2020
61. Reifsnyder, P. C., Doty, R., & Harrison, D. E. (2014). Rapamycin ameliorates nephropathy despite elevating hyperglycemia in a polygenic mouse model of type 2 diabetes, NONcNZO10/LtJ. PLOS One, 9 (12), e114324. doi: 10.1371/journal.pone.0114324
62. Reifsnyder, P. C., Ryzhov, S., Flurkey, K., Anunciado-Koza, R. P., Mills, I., Harrison, D. E., & Koza, R. A. (2018). Cardioprotective effects of dietary rapamycin on adult female C57BLKS/J-Lepr (db) mice. Ann N Y Acad Sci, 1418 (1), 106-117. doi: 10.1111/nyas. 13557
63. Reifsnyder, P. C., Te, A., & Harrison, D. E. (2020). Differential Effects of Rapamycin on Glucose Metabolism in Nine Inbred Strains. J Gerontol A Biol Sci Med Sci, 75 (1), 50-57. doi: 10.1093/gerona/glz157
64. Rich, L., & Whittaker, P. (2005). COLLAGEN AND PICROSIRIUS RED STAINING: A POLARIZED LIGHT ASSESSMENT OF FIBRILLAR HUE AND SPATIAL DISTRIBUTION. Journal of Morphological Sciences. Retrieved from http://www.jms.periodikos.com.br/article/587cb4587f8c9d0d058b460c 65.
Richardson, J. K. (2002). The clinical identification of peripheral neuropathy among older persons. Arch Phys Med Rehabil, 83 (11), 1553-1558. doi: 10.1053/apmr.2002.35656
66. Rittie, L. (2017). Method for Picrosirius Red-Polarization Detection of Collagen Fibers in Tissue Sections. Methods Mol Biol, 1627, 395-407. doi: 10.1007/978-1-4939-7113-8_26
67. Rosada, A., Kassner, U., Weidemann, F., Konig, M., Buchmann, N., Steinhagen-Thiessen, E., & Spira, D. (2020). Hyperlipidemias in elderly patients: results from the Berlin Aging Study II (BASEII), a cross-sectional study. Lipids Health Dis, 19 (1), 92. doi: 10.1186/s12944-020-01277-9
68. Sataranatarajan, K., Ikeno, Y., Bokov, A., Feliers, D., Yalamanchili, H., Lee, H. J., . . . Kasinath, B. S. (2016). Rapamycin Increases Mortality in db/db Mice, a Mouse Model of Type 2 Diabetes. J Gerontol A Biol Sci Med Sci, 71 (7), 850-857. doi: 10.1093/gerona/glv170
69. Schindelin, J., Arganda-Carreras, I., Frise, E., Kaynig, V., Longair, M., Pietzsch, T., Cardona, A. (2012). Fiji: an open-source platform for biological-image analysis. Nat Methods, 9 (7), 676-682. doi: 10.1038/nmeth.2019
70. Selvarani, R., Mohammed, S., & Richardson, A. (2021). Effect of rapamycin on aging and age-related diseases-past and future. Geroscience, 43 (3), 1135-1158. doi: 10.1007/s11357-020-00274-1
71. Spiess, K., & Zorn, T. M. (2007). Collagen types I, III, and V constitute the thick collagen fibrils of the mouse decidua. Microsc Res Tech, 70 (1), 18-25. doi: 10.1002/jemt.20381
72. Sun, K., Tordjman, J., Clement, K., & Scherer, P. E. (2013). Fibrosis and adipose tissue dysfunction. Cell Metab, 18 (4), 470-477. doi: 10.1016/j.cmet.2013.06.016
73. Taetzsch, T., & Valdez, G. (2018). NMJ maintenance and repair in aging. Curr Opin Physiol, 4, 57-64. doi: 10.1016/j.cophys.2018.05.007
74. Tero, B. W., Fortier, B., Soucy, A. N., Paquette, G., & Liaw, L. (2022). Quantification of Lipid Area within Thermogenic Mouse Perivascular Adipose Tissue Using Standardized Image Analysis in FIJI. Journal of vascular research, 59 (1), 43-49. doi: 10.1159/000517178
75. Tran, C. M., Mukherjee, S., Ye, L., Frederick, D. W., Kissig, M., Davis, J. G., . . . Baur, J. A. (2016). Rapamycin Blocks Induction of the Thermogenic Program in White Adipose Tissue. Diabetes, 65 (4), 927-941. doi: 10.2337/db15-0502
76. Tzortzaki, E. G., Koutsopoulos, A. V., Dambaki, K. I., Lambiri, I., Plataki, M., Gordon, M. K., Siafakas, N. M. (2006). Active remodeling in idiopathic interstitial pneumonias: evaluation of collagen types XII and XIV. J Histochem Cytochem, 54 (6), 693-700. doi: 10.1369/jhc.5A6835.2006
77. Valdez, G., Tapia, J. C., Kang, H., Clemenson, G. D., Jr., Gage, F. H., Lichtman, J. W., & Sanes, J. R. (2010). Attenuation of age-related changes in mouse neuromuscular synapses by caloric restriction and exercise. Proc Natl Acad Sci USA, 107 (33), 14863-14868. doi: 10.1073/pnas. 1002220107
78. Van Skike, C. E., Lin, A. L., Roberts Burbank, R., Halloran, J. J., Hernandez, S. F., Cuvillier, J., . . . Galvan, V. (2020). mTOR drives cerebrovascular, synaptic, and cognitive dysfunction in normative aging. Aging Cell, 19 (1), e13057. doi: 10.1111/acel.13057
79. Varghese, M., Griffin, C., McKernan, K., Eter, L., Abrishami, S., & Singer, K. (2020). Female adipose tissue has improved adaptability and metabolic health compared to males in aged obesity. Aging (Albany NY), 12 (2), 1725-1746. doi: 10.18632/aging.102709
80. Verdu, E., Ceballos, D., Vilches, J. J., & Navarro, X. (2000). Influence of aging on peripheral nerve function and regeneration. J Peripher Nerv Syst, 5 (4), 191-208. doi: 10.1046/j. 1529-8027.2000.00026.x
81. Von Bank, H., Kirsh, C., & Simcox, J. (2021). Aging adipose: Depot location dictates age-associated expansion and dysfunction. Ageing Res Rev, 67, 101259. doi: 10.1016/j.arr.2021.101259
82. Wang, P., Loh, K. H., Wu, M., Morgan, D. A., Schneeberger, M., Yu, X., . . . Friedman, J. (2020). A leptin-BDNF pathway regulating sympathetic innervation of adipose tissue. Nature, 583 (7818), 839-844. doi: 10.1038/s41586-020-2527-y
83. Wilkinson, J. E., Burmeister, L., Brooks, S. V., Chan, C. C., Friedline, S., Harrison, D. E., . . . Miller, R. A. (2012). Rapamycin slows aging in mice. Aging Cell, 11 (4), 675-682. doi: 10.1111/j.1474-9726.2012.00832.x
84. Willows, J. W., Blaszkiewicz, M., Lamore, A., Borer, S., Dubois, A. L., Garner, E., Townsend, K. L. (2021). Visualization and analysis of whole depot adipose tissue neural innervation. iScience, 24 (10), 103127. doi: 10.1016/j.isci.2021.103127
85. Willows, J. W., Blaszkiewicz, M., & Townsend, K. L. (2022). A clearing-free protocol for imaging intact whole adipose tissue innervation in mice. STAR Protoc, 3 (1), 101109. doi: 10.1016/j.xpro.2021.101109
86. Willows, J. W., Gunsch, G., Paradie, E., Blaszkiewicz, M., Tonniges, J. R., Pino M. F., Townsend, K. L. (2022). The Presence of Myelinated Nerves and Schwann Cells in White Adipose Tissue: Proximity to Synaptic Vesicle Containing Nerve Terminals and Potential Role in BTBR ob/ob Demyelinating Diabetic Neuropathy. bioRxiv. doi: https://doi.org/10.1101/2022.08.25.505298
87. Wynn, T. A. (2008). Cellular and molecular mechanisms of fibrosis. J Pathol, 214 (2), 199-210. doi: 10.1002/path.2277
88. Yezierski, R. P. (2012). The effects of age on pain sensitivity: preclinical studies. Pain Med, 13 Suppl 2, S27-36. doi: 10.1111/j. 1526-4637.2011.01311.x
89. Yuan, R., Meng, Q., Nautiyal, J., Flurkey, K., Tsaih, S. W., Krier, R., . . . Paigen, B. (2012). Genetic coregulation of age of female sexual maturation and lifespan through circulating IGF1 among inbred mouse strains. Proc Natl Acad Sci USA, 109 (21), 8224-8229. doi: 10.1073/pnas. 1121113109
90. Zhang, L., Ebenezer, P. J., Dasuri, K., Fernandez-Kim, S. O., Francis, J., Mariappan, N., . . . Keller, J. N. (2011). Aging is associated with hypoxia and oxidative stress in adipose tissue: implications for adipose function. Am J Physiol Endocrinol Metab, 301 (4), E599-607. doi: 10.1152/ajpendo.00059.2011
Devigili et al. Clinical diagnosis and management of small fiber neuropathy: an update on best practice. Expert Rev Neurother. 2020 September; 20 (9): 967-980.
Cascio et al. Small Fiber Neuropathy. StatPearls Publishing; 223 January 2023.
Hovaguimian et al. Diagnosis and treatment of pain in small-fiber neuropathy. Curr Pain Headache Rep. 2011 June; 15 (3): 193-200
Lacomis D. Small-fiber neuropathy. Muscle Nerve. 2002 August; 26 (2): 173-88

1. A kit for determining an amount of post-synaptic density protein-95 (PSD-95) present in a subject, wherein the kit comprises a means for detecting PSD-95.

2. The kit of claim 1, wherein a change in the amount of PSD-95 is reflective of a change in peripheral nerve innervation or neuropathy within the subject.

3. The kit of claim 1, wherein the means for detecting PSD-95 is a probe.

4. The kit of claim 3, wherein the probe detects a protein or nucleic acid.

5. The kit of claim 3, wherein the probe is an antibody or nanobody.

6. The kit of claim 3, wherein the probe is a nucleic acid.

7. The kit of claim 1, wherein the means for detecting PSD-95 is a set of primers.

8. The kit of claim 1, wherein the kit further comprises a testing well, a secondary fluorescent or colorimetric detection agent, a wash buffer or extraction buffer, a signal amplification buffer, or a combination thereof.

9. The kit of claim 8, wherein the testing well is a 96-well plate or another well-plate design.

10. The kit of claim 9, wherein a probe is coated onto the base of a testing well or added to the well as part of the assay.

11. The kit of claim 1, wherein the means comprises an Enzyme-linked Immunosorbent Assay (ELISA) or an Enzyme Immunoassay (EIA) or a Polymerase chain reaction (PCR).

12. The kit of claim 11, wherein the PCR comprises, a polymerase enzyme, a nuclease-free water, an MgCl², a deoxynucleotide triphosphate (dNTP), or a combination thereof.

13. The kit of claim 1, wherein the kit further comprises additional means for detecting an additional marker.

14. The kit of claim 13, wherein the additional marker comprises other peripheral nerve markers such as PGP9.5, CGRP, GAP43, tyrosine hydroxylase, or others.

15. A method of treating or preventing peripheral neuropathy or changes to peripheral innervation in response to disease, injury, treatments, or therapies in a subject, the method comprises detecting PSD-95 levels in a sample from the subject, wherein, when the PSD-95 level differs from a control.

16. The method of claim 15, wherein the control is a measurement at a different timepoint from the subject themselves.

17. The method of claim 15, wherein the control is based on PSD-95 level in a subject not experiencing neuropathy or in a standardized control sample.

18. The method of claim 15, wherein the sample is derived from tissue samples, interstitial fluid, or other bodily fluid samples.

19. The method of claim 15, wherein samples are from skin or a subdermal sample such as a skin biopsy punch or an open biopsy from any body surface.

20. The method of claim 15, wherein the method of treatment comprises modulating adipose number and/or size by using a therapy selected from a group comprising calorie-restricted diets/exercise, cholesterol-free diets, low-fat diets, the use of bioactive compounds, pharmacological compounds, or a combination thereof.

21. The method of claim 15, wherein, the method of treating or preventing symptoms in a subject comprises administering a therapeutically effective dose of a PSD-95 modulator to the subject.

22. The method of claim 15, wherein the subject is treated with a pharmacological MMP-13 inhibitor at a therapeutically effective dose.

23. The method of claim 20, wherein after treatment, effectiveness of treatment is assessed.

24. The method of claim 23, wherein PSD-95 levels are measured to determine if treatment was effective.

25. The method of claim 24, wherein PSD-95 levels are measured using the kit of claim 1.

26. The method of claim 25, wherein the effectiveness of the treatment is validated by immunostaining to measure PSD-95 levels in one target selected from the group comprising nerve fiber density, Intradermal N, number of terminal Schwann cells in a neuromuscular junction, adiposity, adipose cell size, adipose tissue inflammation.

27. The method of claim 1, wherein the subject is a human.


SEQUENCE LISTING (58 sequences)

SEQ ID NO: 1 - DNA, 22 bp, synthetic construct
  misc_feature 1..22: note = Description of sequence: VEGFa forward primer
  source 1..22: mol_type = other DNA
aacaaagccagaaaatcactgt

SEQ ID NO: 2 - DNA, 22 bp, synthetic construct
  misc_feature 1..22: note = Description of sequence: VEGFa reverse primer
  source 1..22: mol_type = other DNA
cggatcttggacaaacaaatgc

SEQ ID NO: 3 - DNA, 20 bp, synthetic construct
  misc_feature 1..20: note = Description of sequence: Cd31 forward primer
  source 1..20: mol_type = other DNA
acgctggtgctctatgcaag

SEQ ID NO: 4 - DNA, 22 bp, synthetic construct
  misc_feature 1..22: note = Description of sequence: Cd31 reverse primer
  source 1..22: mol_type = other DNA
tcagttgctgcccattgcatca

SEQ ID NO: 5 - DNA, 18 bp, synthetic construct
  misc_feature 1..18: note = Description of sequence: Il4 forward primer
  source 1..18: mol_type = other DNA
ggtcaacccccagctagt

SEQ ID NO: 6 - DNA, 23 bp, synthetic construct
  misc_feature 1..23: note = Description of sequence: Il4 reverse primer
  source 1..23: mol_type = other DNA
gccgatgatctctctcaagtgat

SEQ ID NO: 7 - DNA, 22 bp, synthetic construct
  misc_feature 1..22: note = Description of sequence: Il6 forward primer
  source 1..22: mol_type = other DNA
tagtccttcctaccccaatttc

SEQ ID NO: 8 - DNA, 21 bp, synthetic construct
  misc_feature 1..21: note = Description of sequence: Il6 reverse primer
  source 1..21: mol_type = other DNA
ttggtccttagccactccttc

SEQ ID NO: 9 - DNA, 23 bp, synthetic construct
  misc_feature 1..23: note = Description of sequence: Il10 forward primer
  source 1..23: mol_type = other DNA
ctatgctgcctgctcttactgac

SEQ ID NO: 10 - DNA, 21 bp, synthetic construct
  misc_feature 1..21: note = Description of sequence: Il10 reverse primer
  source 1..21: mol_type = other DNA
cggagagaggtacaaacgagg

SEQ ID NO: 11 - DNA, 19 bp, synthetic construct
  misc_feature 1..19: note = Description of sequence: Il13 forward primer
  source 1..19: mol_type = other DNA
cctggctcttgcttgcctt

SEQ ID NO: 12 - DNA, 21 bp, synthetic construct
  misc_feature 1..21: note = Description of sequence: Il13 reverse primer
  source 1..21: mol_type = other DNA
ggtcttgtgtgatgttgctca

SEQ ID NO: 13 - DNA, 22 bp, synthetic construct
  misc_feature 1..22: note = Description of sequence: Sox10 forward primer
  source 1..22: mol_type = other DNA
agatccagttccgtgtcaataa

SEQ ID NO: 14 - DNA, 19 bp, synthetic construct
  misc_feature 1..19: note = Description of sequence: Sox10 reverse primer
  source 1..19: mol_type = other DNA
gcgagaagaaggctaggtg

SEQ ID NO: 15 - DNA, 19 bp, synthetic construct
  misc_feature 1..19: note = Description of sequence: Mpz forward primer
  source 1..19: mol_type = other DNA
gtcaagtcccccagtagaa

SEQ ID NO: 16 - DNA, 19 bp, synthetic construct
  misc_feature 1..19: note = Description of sequence: Mpz reverse primer
  source 1..19: mol_type = other DNA
aggagcaagaggaaagcac

SEQ ID NO: 17 - DNA, 16 bp, synthetic construct
  misc_feature 1..16: note = Description of sequence: PSD95 forward primer
  source 1..16: mol_type = other DNA
ggacattcaggccaag

SEQ ID NO: 18 - DNA, 20 bp, synthetic construct
  misc_feature 1..20: note = Description of sequence: PSD95 reverse primer
  source 1..20: mol_type = other DNA
tcccgtagaggtggctgttg

SEQ ID NO: 19 - DNA, 20 bp, synthetic construct
  misc_feature 1..20: note = Description of sequence: Gria2 forward primer
  source 1..20: mol_type = other DNA
gcggtgctaaaatcgaatgc

SEQ ID NO: 20 - DNA, 19 bp, synthetic construct
  misc_feature 1..19: note = Description of sequence: Gria2 reverse primer
  source 1..19: mol_type = other DNA
acagagaggggcaggcagt

SEQ ID NO: 21 - DNA, 20 bp, synthetic construct
  misc_feature 1..20: note = Description of sequence: Syn1 forward primer
  source 1..20: mol_type = other DNA
ccacagggtatgttgtgctg

SEQ ID NO: 22 - DNA, 20 bp, synthetic construct
  misc_feature 1..20: note = Description of sequence: Syn1 reverse primer
  source 1..20: mol_type = other DNA
gcccagatggttcgactaca

SEQ ID NO: 23 - DNA, 20 bp, synthetic construct
  misc_feature 1..20: note = Description of sequence: Syn2 forward primer
  source 1..20: mol_type = other DNA
gccaccaggttaagctctga

SEQ ID NO: 24 - DNA, 18 bp, synthetic construct
  misc_feature 1..18: note = Description of sequence: Syn2 reverse primer
  source 1..18: mol_type = other DNA
ttccaggaaggccaaggt

SEQ ID NO: 25 - DNA, 22 bp, synthetic construct
  misc_feature 1..22: note = Description of sequence: Syp forward primer
  source 1..22: mol_type = other DNA
tgacttcaggactcaacacctc

SEQ ID NO: 26 - DNA, 20 bp, synthetic construct
  misc_feature 1..20: note = Description of sequence: Syp reverse primer
  source 1..20: mol_type = other DNA
caggagctggttgcttttct

SEQ ID NO: 27 - DNA, 20 bp, synthetic construct
  misc_feature 1..20: note = Description of sequence: Atp1 forward primer
  source 1..20: mol_type = other DNA
cggttcaggtctctccactc

SEQ ID NO: 28 - DNA, 20 bp, synthetic construct
  misc_feature 1..20: note = Description of sequence: Atp1 reverse primer
  source 1..20: mol_type = other DNA
tgacgcctcacttgagaatg

SEQ ID NO: 29 - DNA, 20 bp, synthetic construct
  misc_feature 1..20: note = Description of sequence: Uqpcr forward primer
  source 1..20: mol_type = other DNA
ggggtgaccctgagtattga

SEQ ID NO: 30 - DNA, 20 bp, synthetic construct
  misc_feature 1..20: note = Description of sequence: Uqpcr reverse primer
  source 1..20: mol_type = other DNA
atgtaaggcacccagtccag

SEQ ID NO: 31 - DNA, 20 bp, synthetic construct
  misc_feature 1..20: note = Description of sequence: Cox5b forward primer
  source 1..20: mol_type = other DNA
cagaagggactggacccata

SEQ ID NO: 32 - DNA, 20 bp, synthetic construct
  misc_feature 1..20: note = Description of sequence: Cox5b reverse primer
  source 1..20: mol_type = other DNA
ataacacaggggctcagtgg

SEQ ID NO: 33 - DNA, 20 bp, synthetic construct
  misc_feature 1..20: note = Description of sequence: Ndufa1 forward primer
  source 1..20: mol_type = other DNA
gcggagccagatgttaaaaa

SEQ ID NO: 34 - DNA, 20 bp, synthetic construct
  misc_feature 1..20: note = Description of sequence: Ndufa1 reverse primer
  source 1..20: mol_type = other DNA
ccatccaccatctgacactg

SEQ ID NO: 35 - DNA, 24 bp, synthetic construct
  misc_feature 1..24: note = Description of sequence: Col1 forward primer
  source 1..24: mol_type = other DNA
ttctcctggcaaagacggactcaa

SEQ ID NO: 36 - DNA, 23 bp, synthetic construct
  misc_feature 1..23: note = Description of sequence: Col1 reverse primer
  source 1..23: mol_type = other DNA
ggaagctgaagtcataaccgcca

SEQ ID NO: 37 - DNA, 23 bp, synthetic construct
  misc_feature 1..23: note = Description of sequence: Col2 forward primer
  source 1..23: mol_type = other DNA
aagggacaccgaggtttcactgg

SEQ ID NO: 38 - DNA, 21 bp, synthetic construct
  misc_feature 1..21: note = Description of sequence: Col2 reverse primer
  source 1..21: mol_type = other DNA
gggcctgtttctcctgagcgt

SEQ ID NO: 39 - DNA, 20 bp, synthetic construct
  misc_feature 1..20: note = Description of sequence: Col3 forward primer
  source 1..20: mol_type = other DNA
gcccacagccttctacacct

SEQ ID NO: 40 - DNA, 19 bp, synthetic construct
  misc_feature 1..19: note = Description of sequence: Col3 reverse primer
  source 1..19: mol_type = other DNA
gccagggtcaccatttctc

SEQ ID NO: 41 - DNA, 21 bp, synthetic construct
  misc_feature 1..21: note = Description of sequence: Col4 forward primer
  source 1..21: mol_type = other DNA
ctcactgtggatcggctattc

SEQ ID NO: 42 - DNA, 22 bp, synthetic construct
  misc_feature 1..22: note = Description of sequence: Col4 reverse primer
  source 1..22: mol_type = other DNA
cgcttctaaactcttccagaca

SEQ ID NO: 43 - DNA, 20 bp, synthetic construct
  misc_feature 1..20: note = Description of sequence: Col5 forward primer
  source 1..20: mol_type = other DNA
cttcgccgctactcctgttc

SEQ ID NO: 44 - DNA, 22 bp, synthetic construct
  misc_feature 1..22: note = Description of sequence: Col5 reverse primer
  source 1..22: mol_type = other DNA
ccctgagggcaaattgtgaaaa

SEQ ID NO: 45 - DNA, 21 bp, synthetic construct
  misc_feature 1..21: note = Description of sequence: Col6 forward primer
  source 1..21: mol_type = other DNA
acagtgacgaggtggagatca

SEQ ID NO: 46 - DNA, 20 bp, synthetic construct
  misc_feature 1..20: note = Description of sequence: Col6 reverse primer
  source 1..20: mol_type = other DNA
gatagcgcagtcggtgtagg

SEQ ID NO: 47 - DNA, 21 bp, synthetic construct
  misc_feature 1..21: note = Description of sequence: Ppia forward primer
  source 1..21: mol_type = other DNA
caaatgctggaccaaacacaa

SEQ ID NO: 48 - DNA, 19 bp, synthetic construct
  misc_feature 1..19: note = Description of sequence: Ppia reverse primer
  source 1..19: mol_type = other DNA
aagaccacatgcttgccat

SEQ ID NO: 49 - DNA, 23 bp, synthetic construct
  misc_feature 1..23: note = Description of sequence: Human PSD-95 forward primer
  source 1..23: mol_type = other DNA
tgagatcagtcatagcagctact

SEQ ID NO: 50 - DNA, 20 bp, synthetic construct
  misc_feature 1..20: note = Description of sequence: Human PSD-95 reverse primer
  source 1..20: mol_type = other DNA
cttcctcccctagcaggtcc

SEQ ID NO: 51 - protein, 45 aa, Homo sapiens
  REGION 1..45: note = Description of sequence: Human PSD-95 amino acids 135-180
ALKEAGSIVRLYVMRRKPPAEKVMEIKLIKGPKGLGFSIAGGVGN

SEQ ID NO: 52 - protein, 45 aa, Mus musculus
  REGION 1..45: note = Description of sequence: Mouse PSD-95 amino acids 135-180
ALKEAGSIVRLYVMRRKPPAEKIIEIKLIKGPKGLGFSIAGGVGN

SEQ ID NO: 53 - protein, 45 aa, Homo sapiens
  REGION 1..45: note = Description of sequence: Human PSD-95 amino acids 450-495
ALSFRFGDVLHVIDASDEEWWQARRVHSDSETDDIGFIPSKRRVE

SEQ ID NO: 54 - protein, 45 aa, Mus musculus
  REGION 1..45: note = Description of sequence: Mouse PSD-95 amino acids 450-495
ALSFHFGDVLHVIDASDEEWWQARRVHSDSETDDIGFIPSKRRVE

SEQ ID NO: 55 - protein, 58 aa, Mus musculus
  REGION 1..58: note = Description of sequence: Mouse DLG4 amino acids 1-58
MDCLCIVTTKKYRYQDEDTPPLEHSPAHLPNQANSPPVIVNTDTLEAPGYELQVNGTE

SEQ ID NO: 56 - protein, 58 aa, Homo sapiens; identical to SEQ ID NO: 55
  REGION 1..58: note = Description of sequence: Human DLG4 amino acids 1-58

SEQ ID NO: 57 - protein, 244 aa, Homo sapiens
  REGION 1..244: note = Description of sequence: Mouse DLG4 amino acids 1-305
EYEEITLERGNSGLGFSIAGGTDNPHIGDDPSIFITKIIPGGAAAQDGRTRVNDSILFVNEVDVREVTHSAAVEALKEAGSIVRLYVMRRKPPAEKVMEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEE

SEQ ID NO: 58 - protein, 244 aa, Mus musculus
  REGION 1..244: note = Description of sequence: Human DLG4 amino acids 1-305
EYEEITLERGNSGLGFSIAGGTDNPHIGDDPSIFITKIIPGGAAAQDGRTRVNDSILFVNEVDVREVTHSAAVEALKEAGSIVRLYVMRRKPPAEKIIEIKLIKGPKGLGFSIAGGVGNQHIPGDNSIYVTKIIEGGAAHKDGRLQIGDKILAVNSVGLEDVMHEDAVAALKNTYDVVYLKVAKPSNAYLSDSYAPPDITTSYSQHLDNEISHSSYLGTDYPTAMTPTSPRRYSPVAKDLLGEE